(19) United States
(12) Patent Application Publication
(10) Pub. No.: US 2008/0009512 A1
(43) Pub. Date: Jan. 10, 2008

(54) Tetrahydroquinolones and Aza-Analogues Thereof for Use as Dpp-IV Inhibitors in the Treatment of Diabetes

(75) Inventors: Alan Martin Birch, Macclesfiled (GB); Paul David Kemmitt, Macclesfield (GB); Nathaniel George Martin, Macclesfield (GB); Richard Andrew Ward, Macclesfield (GB)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 11/569,943

(22) PCT Filed: Jun. 14, 2005
(86) PCT No.: PCT/GB05/02349

§ 371 Date: Dec. 1, 2006

(30) Foreign Application Priority Data
Jun. 16, 2004 (GB) 0413389.8

(51) Int. Cl. A61K 31/47; A61K 31/4375; A61P 3/10; C07D 215/60; C07D 471/04

(52) U.S. Cl. 514/300; 514/312; 546/122; 546/155; 546/158

(57) ABSTRACT

Compound of formula (I) or a pharmaceutically-acceptable salt thereof, formula (I) wherein Ar is optionally substituted phenyl; R¹ is selected from: formula a) or b) (wherein is a single or double bond); R⁵, R₆, R₇ and R⁸ are for or alkyl; R⁴ is selected from hydrogen, (3-4C)cycloalkyl and optionally substituted (1-4C)alkyl; R¹⁰ is for example selected from hydrogen, (1-4C)alkyl, (3-6C)cycloalkyl(1-4C)alkyl, hydroxy(1-4C)alkyl, (1-4C)alkoxy, aryl(1-4C)alkyl; Y is carbon and Ring A is optionally substituted phenylene; or each Y may independently be carbon or nitrogen and Ring A is optionally substituted 5- or 6-membered, heteroarylene ring; R¹¹ is selected from hydrogen and optionally substituted phenyl; p is independently at each occurrence 0, 1 or 2; are described. Processes for making such compounds and their use as DPP-IV inhibitors in the treatment of diabetes are also described.

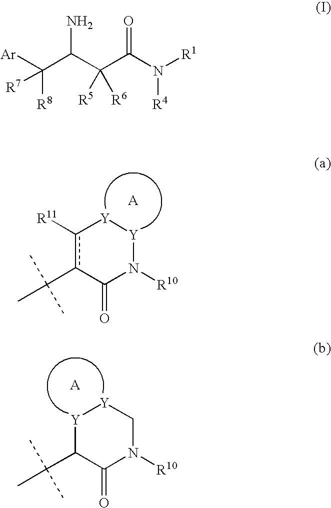

The present invention relates to compounds which inhibit dipeptidyl peptidase IV (DPP-IV) activity, processes for their preparation, pharmaceutical compositions containing them as the active ingredient, methods for the treatment of disease states associated with DPP-IV activity, to their use as medicaments and to their use in the manufacture of medicaments for use in the inhibition of DPP-IV in warm-blooded animals such as humans. In particular this invention relates to compounds useful for the treatment of diabetes mellitus in warm-blooded animals such as humans, more particularly to the use of these compounds in the manufacture of medicaments for use in the treatment of diabetes mellitus in warm-blooded animals such as humans.

DPP-IV is a serine protease found throughout the body, which degrades and regulates the activity of several regulatory peptides in man including glucagon-like peptide-1 (GLP-1), GLP-2, GHRH (growth hormone releasing hormone) and GIP (glucagon interacting peptide). GLP-1 is a peptide hormone which is released from the intestinal tract wall into the bloodstream in response to a meal and strongly influences post-prandial glucose metabolism. As post-prandial glucose levels rise, GLP-1 acts directly on pancreatic β-cells to augment insulin release and also promote new insulin biosynthesis. Simultaneously, GLP-1 delays gastric emptying, further suppressing meal-related rise in plasma glucose. It has been shown (Rachman, J. et al, (1997), Diabetologia, 40, 205-211; Nauck, M. A. et al, (1996), Diabetologia, 39,1546-1553; Gutniak, M. K. et al, (1994), Diabetes Care, 17, 1039-1045; Rachman J. et al, (1996) Diabetes, 45, 1524-1530) that GLP-1 administration either subcutaneously or by intravenous infusion improves glucose tolerance in diabetic patients, however daily administration of GLP-1 is not generally considered to be a desirable form of therapy.

DPP-IV degrades GLP-1 circulating in the bloodstream and inhibition of DPP-IV activity causes an increase in the half life, and therefore activity, of GLP-1. Additionally DPP-IV inhibitors have beneficial effects on pancreatic failure: Ribel U. et al ((2001) Diabetologia, 44, A192, 738) described how the DPP-IV inhibitor valine pyrrolidide (VP) promoted differentiation of new beta cells in 60% pancreatectomised rats. Therefore, administration of a DPP-WV inhibitor should result in prolongation of endogenous GLP-1 activity and thus potentially in a clinically significant lowering of diabetic hyperglycemia. A DPP-IV inhibitor may potentially be useful for the prevention, delay or treatment of Type 2 (non-insulin dependent) diabetes mellitus.

Novel DPP-IV inhibitors have been described in the art. Many are 2-cyanopyrrolidine derivatives with a significant range of substituents bonded to the ring nitrogen (see for example WO 98/19998, WO 00/34241, WO 01/96295, WO 01/40180), or contain this structure (see for example WO 00/168603 which discloses cyclopropyl fused cyano pyrrolidines). Others are cyanothiazolidines (see for example US 00/6110949, US 00/6107317, WO 99/61431), also with a variety of substituents bonded to the ring nitrogen. Still others contain pyrrolidine, piperidine, or morpholine rings which may contain substituents on the ring carbon atoms other than cyano groups (see for example WO 03/000181 and WO 03/000180).

We have surprisingly found a new structural class of DPP-IV inhibitors.

Accordingly, the present invention provides a compound of formula (I) or a pharmaceutically-acceptable salt thereof,

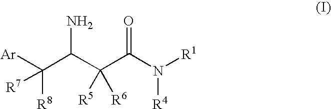

wherein:

Ar is phenyl optionally substituted with 1, 2, 3, 4 or 5 groups independently selected from R⁹;

R⁹ is selected from halo, (1-2C)alkyl (optionally substituted with 1, 2, 3, 4 or 5 substituents independently selected from halo), hydroxy, methoxy (optionally substituted with 1, 2 or 3 substituents independently selected from halo) and cyano;

R¹ is selected from: 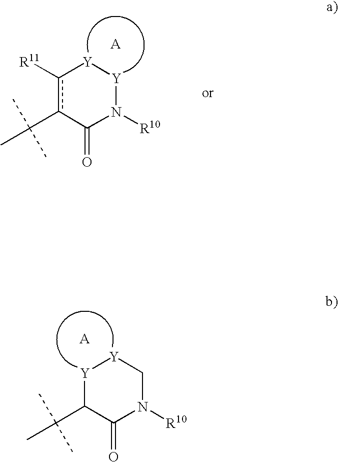
(wherein [symbol] is a single or double bond);

R⁵ and R⁶ are independently selected from hydrogen, hydroxy and (1-4C)alkyl; or

R⁵ and R⁶ together with the carbon to which they are attached form a cyclopropyl ring;

R⁷ and R⁸ are independently selected from hydrogen, hydroxy and (1-4C)alkyl; or

R⁷ and R⁸ together with the carbon to which they are attached form a cyclopropyl ring;

provided that only one of R⁵, R⁶, R⁷ and R⁸ is hydroxy;

R⁴ is selected from hydrogen, (3-4C)cycloalkyl and (1-4C)alkyl (optionally substituted with 1 substituent selected from (3-4C)cycloalkyl, hydroxy, (1-4C)alkoxy, halo and —S(O)p(1-4C)alkyl);

R¹⁰ is selected from hydrogen, (1-4C)alkyl, -(1-4C)alkyl(3-6C)cycloalkyl, hydroxy(1-4C)alkyl, (1-4C)alkoxy, (1-4C)alkoxy(1-4C)alkyl, (1-4C)alkylS(O)p(1-4C)alkyl, aryl(1-4C)alkyl, heteroaryl(1-4C)alkyl, -(1-4C)alkylCONH₂, -(1-4C)alkylCONH(1-4C)alkyl, -(1-4C)alkylCONdi(1-4C)alkyl, -(1-4C)alkylSO₂NH₂, -(1-4C)alkylSO₂NH(1-4C)alkyl, -(1-4C)alkylSO₂Ndi(1-4C)alkyl, -(2-4C)alkylNHCO(1-4C)alkyl, -(2-4C)alkylNHSO₂(1-4C)alkyl, -(1-4C)alkylCO₂H, and -(1-4C)alkylCO₂(1-4C)alkyl;

Y is carbon and Ring A is phenylene; or

each Y may independently be carbon or nitrogen and Ring A is 5- or 6-membered, heteroarylene ring containing 1 or 2 heteroatoms independently selected from O, N and S (but not containing any O—O, O—S or S—S bonds), fused via Y as a ring carbon atom or nitrogen atom (provided that the ring maintains aromaticity);

wherein Ring A is optionally substituted by 1 or 2 substituents independently selected from R²;

R² is independently selected from phenyl, heteroaryl, cyano, halo, (1-4C)alkyl, halo(1-4C)alkoxy, halo(1-4C)alkyl, dihalo(1-4C)alkyl, trifluoromethyl, pentafluoroethyl, (1-4C)alkoxy, hydroxy, amino, (1-4C)alkylamino, di(1-4C)alkylamino, —CONH₂, —CONH(1-4C)alkyl, —CONdi(1-4C)alkyl, —NHCO(1-4C)alkyl, —S(O)₂NH₂, —SO₂NH(1-4C)alkyl, —SO₂Ndi(1-4C)alkyl, —SO2(1-4C)alkyl, —NHSO₂(1-4C)alkyl, —CO(1-4C)alkyl, —CO₂(1-4C)alkyl, —OCO(1-4C)alkyl, (3-5C)cycloalkyl, -(1-4C)alkyl(3-5C)cycloalkyl, halo(3-5C)cycloalkoxy, halo(3-5C)cycloalkyl, dihalo(3-5C)cycloalkyl, trihalo(3-5C)cycloalkyl, (3-5C)cycloalkoxy, (3-5C)cycloalkylamino, —CONH(3-5C)cycloalkyl, —NHCO(3-5C)cycloalkyl, —SO₂NH(3-5C)cycloalkyl, —SO₂(3-5C)cycloalkyl, —NHSO₂(3-5C)cycloalkyl, —CO(3-5C)cycloalkyl, —CO₂(3-5C)cycloalkyl and —OCO(3-5C)cycloalkyl;

R¹¹ is selected from hydrogen and phenyl optionally substituted by 1, 2 or 3 substitutents independently selected from halo, (1-4C)alkyl, (1-4C)alkoxy, halo(1-4C)alkyl, halo(1-4C)alkoxy, (3-6C)cycloalkyl, (3-6C)cycloalkoxy, -(1-4)alkyl(3-6C)cycloalkyl, -(1-4C)alkoxy(3-6C)cycloalkyl, —S(O)p(1-4C)alkyl and —OSO₂(1-4C)alkyl;

p is independently at each occurrence 0, 1 or 2.

In another aspect of the invention, there is provided a compound of formula (I) or a pharmaceutically-acceptable salt thereof as hereinbefore defined, wherein:

R¹⁰ is selected from hydrogen, (1-4C)alkyl, hydroxy(1-4C)alkyl, (1-4C)alkoxy(1-4C)alkyl, (1-4C)alkylS(O)p(1-4C)alkyl, aryl(1-4C)alkyl, heteroaryl(1-4C)alkyl, -(1-4C)alkylCONH₂, -(1-4C)alkylCONH(1-4C)alkyl, -(1-4C)alkylCONdi(1-4C)alkyl, -(1-4C)alkylSO₂NH₂, -(1-4C)alkylSO₂NH(1-4)alkyl, -(1-4C)alkylSO₂Ndi(1-4C)alkyl, -(2-4C)alkylNHCO(1-4C)alkyl, -(2-4C)alkylNHSO₂(1-4C)alkyl, -(1-4C)alkylCO₂H, and -(1-4C)alkylCO₂(1-4C)alkyl; and

R¹¹ is hydrogen.

For the avoidance of doubt it is to be understood that where in this specification a group is qualified by ‘hereinbefore defined’ or ‘defined hereinbefore’ the said group encompasses the first occurring and broadest definition as well as each and all of the particular definitions for that group.

It is to be understood that where substituents contain two substituents on an alkyl chain, in which both are linked by a heteroatom (for example two alkoxy substituents), then these two substituents are not substituents on the same carbon atom of the alkyl chain.

In this specification the term “alkyl” includes both straight and branched chain alkyl groups but references to individual alkyl groups such as “propyl” are specific for the straight chain version only. An analogous convention applies to other generic terms. Unless otherwise stated the term “alkyl” advantageously refers to chains with 1-6 carbon atoms, preferably 1-4 carbon atoms.

In this specification the term “alkoxy” means an alkyl group as defined hereinbefore linked to an oxygen atom.

It is to be understood that optional substituents on any group may be attached to any available atom as appropriate unless otherwise specified, including heteroatoms provided that they are not thereby quaternised.

Within this specification composite terms are used to describe groups comprising more that one functionality such as -(1-6C)alkylNHSO₂(1-6C)alkyl. Such terms are to be interpreted in accordance with the meaning which is understood by a person skilled in the art for each component part. For example -(1-6)alkylNHSO₂(1-6C)alkyl includes -methylaminosulphonlylmethyl, -methylaminosulphonylethyl, -ethylaminosulphonylmethyl, and -propylaminosulphonylbutyl.

Where optional substituents are chosen from “0, 1, 2 or 3” groups it is to be understood that this definition includes all substituents being chosen from one of the specified groups or the substituents being chosen from two or more of the specified groups. An analogous convention applies to substituents chose from “0, 1 or 2” groups and “1 or 2” and any other analogous groups.

Substituents may be present at any suitable position on, for example, an alkyl group. Therefore, hydroxy substituted (1-6C)alkyl includes hydroxymethyl, 1-hydroxyethyl, 2-hydroxyethyl and 3-hydroxypropyl.

Examples of (1-4C)alkyl include methyl, ethyl, propyl and isopropyl; examples of (1-6C)alkyl include methyl, ethyl, propyl, isopropyl, t-butyl, pentyl, iso-pentyl, 1-2-dimethylpropyl and hexyl; examples of (1-3C)alkyl include methyl, ethyl, propyl and isopropyl; examples of (3-4C)cycloalkyl are cyclopropyl and cyclobutyl; examples of (3-5C)cycloalkyl include (3-4C)cycloalkyl and cyclopentyl; examples of (3-6C)cycloalkyl include (3-5C)cycloalkyl and cyclohexyl; examples of -(1-4C)alkyl(3-4C)cycloalkyl include cyclopropylmethyl, cyclopropylethyl, cyclobutylmethyl, cyclobutylethyl, cyclopropylpropyl and cyclopropylbutyl; examples of -(1-4C)alkyl(3-5C)cycloalkyl include -(1-4C)alkyl(3-4C)cycloalkyl and cyclopentylmethyl; examples of -(1-4C)alkyl(3-6C)cycloalkyl include -(1-4C)alkyl(3-5C)cycloalkyl and cyclohexylmethyl; examples of -(1-4C)alkoxy(3-6C)cycloalkyl include cyclopropylmethoxy, cyclopropylethoxy, cyclobutylmethoxy, cyclobutylethoxy, cyclopropylpropoxy, cyclopropylbutoxy and cyclohexylmethoxy; examples of (1-6C)alkoxy include methoxy, ethoxy, propoxy, isopropoxy, tert-butoxy and pentoxy; examples of (1-4C)alkoxy include methoxy, ethoxy, propoxy, isopropoxy and tert-butoxy; examples of (1-4C)alkoxy(1-4C)alkyl include methoxymethyl, ethoxymethyl, methoxyethyl, propoxymethyl, isopropoxymethyl and tert-butoxybutyl; examples of (3-5C)cycloalkoxy include cyclopropoxy, cyclobutoxy, and cyclopentoxy; examples of (3-6C)cycloalkoxy include(3-5C)cycloalkyloxy and cyclohexyloxy; examples of halo are chloro, bromo and fluoro; examples of halo(1-4C)alkyl include chloromethyl, fluoroethyl and fluoromethyl; examples of dihalo(1-4C)alkyl include dichloromethyl, difluoromethyl, 1,2-difluoroethyl and 1,1-difluoroethyl; examples of halo(1-4C)alkoxy include chloromethoxy, fluoroethoxy and fluoromethoxy; examples of halo(3-5C)cycloalkoxy include fluorocyclopropoxy, chlorocyclopropoxy, fluorocyclobutoxy, and fluorocyclopentoxy; examples of halo(3-5C)cycloalkyl include fluorocyclopropyl, chlorocyclopropyl, fluorocyclobutyl, and fluorocyclopentyl; examples of dihalo(3-5C)cycloalkyl include difluorocyclopropyl, dichlorocyclopropyl, fluorochlorocyclopropyl, difluorocyclobutyl, and difluorocyclopentyl; examples of trihalo(3-5C)cycloalkyl include trifluorocyclopropyl, trichlorocyclopropyl, difluorochlorocyclopropyl, trifluorocyclobutyl, and trifluorocyclopentyl; examples of fluoro(3-5C)cycloalkyl include fluorocyclopropyl, fluorocyclobutyl, and fluorocyclopentyl;examples of difluoro(3-5C)cycloalkyl include 2,3-difluorocyclopropyl, 2,2-difluorocyclopropyl, difluorocyclobutyl, and difluorocyclopentyl; examples of trifluoro(3-5C)cycloalkyl include trifluorocyclopropyl, trifluorocyclobutyl, and trifluorocyclopentyl; examples of hydroxy(1-6C)alkyl include hydroxy(1-4C)alkyl such as hydroxy methyl, 1-hydroxyethyl, 2-hydroxyethyl and 3-hydroxybutyl; examples of hydroxy(1-6C)alkoxy include hydroxy(1-4C)alkoxy such as hydroxymethoxy, 2-hydroxyethoxy and 3-hydroxybutoxy; examples of carboxy(1-4C)alkyl and -(1-4C)alkylCO₂H include carboxymethyl, 1-carboxyethyl, 2-carboxyethyl, 2-carboxypropyl and 3-carboxypropyl; examples of (1-6C)alkylamino include (1-4C)alkylamino such as methylamino, ethylamino and propylamino; examples of di-((1-6C)alkyl)amino include di-(1-4C)alkylamino such as dimethylamino, N-ethyl-N-methylamino, diethyl amino, N-methyl-N-propylamino and di-isopropylamino; examples of (3-5C)cycloalkylamino include cyclopropylamino, cyclobutylamino and cyclopentylamino; examples of —CO(1-6C)alkyl include —CO(1-4C)alkyl such as methylcarbonyl, ethylcarbonyl, propylcarbonyl, iso-propylcarbonyl and tert-butylcarbonyl; examples of —CO(3-5C)cycloalkyl include cyclopropylcarbonyl, cyclobutylcarbonyl and cyclopentylcarbonyl; examples of —OCO(1-6C)alkyl include —OCO(1-4C)alkyl such as methylcarbonyloxy, ethylcarbonyloxy, propylcarbonyloxy, iso-propylcarbonyloxy and tert-butylcarbonyloxy; examples of —OCO(3-5C)cycloalkyl include cyclopropylcarbonyloxy, cyclobutylcarbonyloxy and cyclopentylcarbonyloxy; examples of —CO₂(1-6C)alkyl include -CO₂(1-4C)alkyl such as methoxycarbonyl, ethoxycarbonyl, propoxycarbonyl, iso-propoxycarbonyl and tert-butoxycarbonyl; examples of —CO₂(3-5C)cycloalkyl include cyclopropoxycarbonyl, cyclobutoxycarbonyl and cyclopentoxycarbonyl; examples of —SO₂(1-4C)alkyl include methylsulfonyl, ethylsulfonyl, propylsulfonyl, isopropylsulfonyl and butylsulfonyl; examples of —OSO₂(1-4C)alkyl include methylsulfonyloxy, ethylsulfonyloxy, propylsulfonyloxy, isopropylsulfonyloxy and butylsulfonyloxy; examples of —SO₂(3-5C)cycloalkyl include cyclopropylsulfonyl, cyclobutylsulfonyl and cyclopentylsulfonyl; examples of —NHCO(1-6C)alkyl include —NHCO(1-4C)alkyl such as methylcarbonylamino, ethylcarbonylamino, propylcarbonylamino, iso-propylcarbonylamino and tert-butylcarbonylamino; examples of —NHCO(3-5C)cycloalkyl include cyclopropylcarbonylamino, cyclobutylcarbonylamino and cyclopentylcarbonylamino; examples of —CONH(1-6C)alkyl include —CONH(1-4C)alkyl such as methylaminocarbonyl, ethylaminocarbonyl, propylaminocarbonyl, iso-propylaminocarbonyl and tert-butylaminocarbonyl; examples of —CONH(3-5C)cycloalkyl include cyclopropylaminocarbonyl, cyclobutylaminocarbonyl and cyclopentylaminocarbonyl; examples of —CONdi(1-6C)alkyl include —CONdi(1-4C)alkyl such as dimethylaminocarbonyl, N-methyl-N-ethylaminocarbonyl, diethylaminocarbonyl, N-methyl-N-propylaminocarbonyl and di-isopropylaminocarbonyl; examples of —S(O)p(1-4C)alkyl (wherein p is 0, 1 or 2) include methylthio, methylsulfinyl, methylsulfonyl, ethylthio, ethylsulfinyl, ethylsulfonyl, propylthio, butylthio; examples of —SO₂NH(1-6C)alkyl include —SO₂NH(1-4C)alkyl such as methylaminosulfonyl, ethylaminosulfonyl, propylaminosulfonyl, iso-propylaminosulfonyl and tert-butylaminosulfonyl; examples of —SO₂NH(3-5C)cycloalkyl include cyclopropylaminosulfonyl, cyclobutylaminosulfonyl and cyclopentylaminosulfonyl; examples of —SO₂Ndi(1-6C)alkyl include —SO₂Ndi(1-4C)alkyl such as dimethylaminosulfonyl, N-methyl-N-ethylaminosulfonyl, diethylaminosulfonyl, N-methyl-N-propylaminosulfonyl and di-isopropylaminosulfonyl; examples of —NHSO₂(1-6C)alkyl include —NHSO₂(1-4C)alkyl such as methylsulfonylamino, ethylsulfonylamino, propylsulfonylamino, iso-propylsulfonylamino and tert-butylsulfonylamino; examples of —NHSO₂(3-5C)cycloalkyl include cyclopropylsulfonylamino, cyclobutylsulfonylamino and cyclopentylsulfonylamino; examples of —(1-6C)alkylCO(1-6C)alkyl include -(1-4C)alkylCO(1-4C)alkyl such as methylcarbonylmethyl, methylcarbonylbutyl, ethylcarbonymethyl, propylcarbonylbutyl, iso-propylcarbonylmethyl and tert-butylcarbonylmethyl; examples of -(1-6C)alkylOCO(1-6C)alkyl include -(1-4C)alkylOCO(1-4C)alkyl such as methylcarbonyloxymethyl, methylcarbonyloxybutyl, ethylcarbonyloxymethyl, propylcarbonyloxybutyl, iso-propylcarbonyloxymethyl and tert-butylcarbonyloxymethyl; examples of -(1-6C)alkylCO₂(1-6C)alkyl include -(1-4C)alkylCO₂(1-4C)alkyl such as methoxycarbonylmethyl, methyoxycarbonylbutyl, ethoxycarbonylmethyl, propoxycarbonylmethyl, iso-propoxycarbonylmethyl and tert-butoxycarbonylmethyl; examples of (1-4C)alkylS(O)p(1-4C)alkyl include methylthiomethyl, methylsulfinylmethyl, methylsulfonylmethyl, methylthioethyl, methylsulfinylethyl, methylsulfonylethyl, ethylthiomethyl, ethylsulfinylmethyl and ethylsulfonylmethyl; examples of -(1-6C)alkylNHCO(1-6C)alkyl include -(1-4C)alkylNHCO(1-4C)alkyl such as methylcarbonylaminomethyl, methylcarbonylaminopropyl, ethylcarbonylaminomethyl, propylcarbonylaminomethyl, iso-propylcarbonylaminomethyl and tert-butylcarbonylaminomethyl; examples of -(2-4C)alkylNHCO(1-4C)alkyl include methylcarbonylaminoethyl, methylcarbonylaminopropyl, ethylcarbonylaminoethyl, propylcarbonylaminoethyl, iso-propylcarbonylaminoethyl and tert-butylcarbonylaminoethyl; examples of -(1-6C)alkylCONH(1-6C)alkyl include -(1-4C)alkylCONH(1-4C)alkyl such as methylaminocarbonylmethyl, methylaminocarbonylpropyl, ethylaminocarbonylmethyl, propylaminocarbonylmethyl, iso-propylaminocarbonylmethyl and tert-butylaminocarbonylmethyl; examples of -(1-6C)alkylCONH₂ include -(1-4C)alkylCONH₂ such as carbamoylmethyl, carbamoylethyl, carbamoylpropyl and carbamoylbutyl; examples of -(1-6C)alkylCONdi(1-6C)alkyl include -(1-4C)alkylCONdi(1-4C)alkyl such as dimethylaminocarbonylmethyl, dimethylaminocarbonylpropyl, N-methyl-N-ethylaminocarbonylmethyl, diethylaminocarbonylmethyl, N-methyl-N-propylaminocarbonylmethyl and di-isopropylaminocarbonylmethyl; examples of -(1-6C)alkylNH(1-6C)alkyl include -(1-4C)alkylNH(l -4C)alkyl such as methylaminomethyl, methylaminopropyl, ethylaminomethyl, propylaminomethyl, iso-propylaminomethyl and tert-butylaminomethyl; examples of -(1-6C)alkylNdi(1-6C)alkyl include -(1-4C)alkylNdi(1-4C)alkyl such as dimethylaminomethyl, dimethylaminopropyl, N-methyl-N-ethylaminomethyl, diethylaminomethyl, N-methyl-N-propylaminomethyl and di-isopropylaminomethyl; examples of -(1-6C)alkylSO₂NH₂ include -(1-4C)alkylSO₂NH₂ such as sulfamoylmethyl, sulfamoylethyl, sulfamoylpropyl and sulfamoylbutyl; examples of -(1-6C)alkylSO₂NH(1-6C)alkyl include -(1-4C)alkylSO₂NH(1-4C)alkyl such as methylaminosulfonylmethyl, methylaminosulfonylpropyl, ethylaminosulfonylmethyl, propylaminosulfonylmethyl, iso-propylaminosulfonylmethyl and tert-butylaminosulfonylmethyl; examples of -(1-6C)alkylSO₂Ndi(1-6C)alkyl include -(1-4C)alkylSO₂Ndi(1-4C)alkyl such as dimethylaminosulfonylmethyl, dimethylaminosulfonylpropyl, N-methyl-N-ethylaminosulfonylmethyl, diethylaminosulfonylmethyl, N-methyl-N-propylaminosulfonylmethyl and di-isopropylaminosulfonylmethyl; examples of -(1-6C)alkylNHSO₂(1-6C)alkyl include -(1-4C)alkylNHSO₂(1-4C)alkyl such as methylsulfonylaminomethyl, methylsulfonylaminopropyl, ethylsulfonylaminomethyl, propylsulfonylaminomethyl, iso-propylsulfonylaminomethyl and tert-butylsulfonylaminomethyl; examples of -(2-4C)alkylNHSO2(1-4C)alkyl include methylsulfonylaminoethyl, methylsulfonylaminopropyl, ethylsulfonylaminoethyl, propylsulfonylaminoethyl, iso-propylsulfonylaminoethyl and tert-butylsulfonylaminoethyl.

Heteroarylene is a diradical of a heteroaryl group.

Particular values for Ring A as a heteroarylene ring include, for example furylene, pyrrolylene, thienylene, pyrazolylene, imidazolylene, pyridylene, pyrimidylene, pyrazinylene, pyridazinylene, oxazolylene, isoxazolylene, oxazinylene, thiazolylene, isothiazolylene. A more particular value for Ring A as a heteroarylene ring is pyridylene.

For the avoidance of doubt, the following illustrate some possible values for R¹ where ring A is heteroarylene ring:

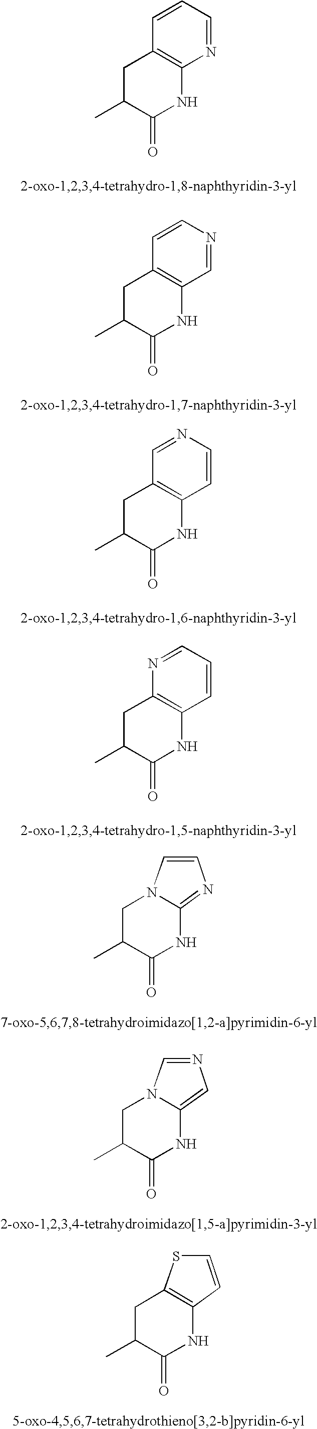

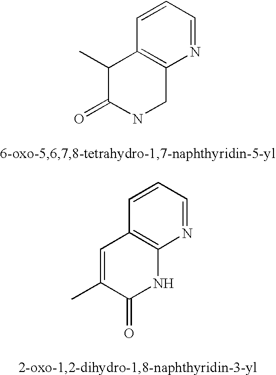

Examples of aryl are optionally substituted phenyl and optionally substituted naphthyl.

Examples of aryl(1-4C)alkyl are optionally substituted benzyl, optionally substituted phenethyl, optionally substituted naphthylmethyl and optionally substituted naphthylethyl.

Suitable optional substituents for phenyl and aryl groups (including where the aryl group is attached to another group such as in aryl(1-4C)alkyl) are, unless otherwise defined, 1, 2 or 3 substituents independently selected from halo, cyano, nitro, amino, hydroxy, (1-4C)alkyl (optionally substituted with 1, 2, 3, 4 or 5 halo), (1-4C)alkoxy (optionally substituted with 1, 2, 3, 4 or 5 halo), —S(O)p(1-4C)alkyl (wherein p is 0, 1 or 2), (1-4C)alkylamino and di-(1-4C)alkylamino. Further suitable optional substituents for aryl groups are heteroaryl, —OCO(1-4C)alkyl, —CO₂(1-4C)alkyl, —NHCO(1-4C)alkyl, —CONH(1-4C)alkyl, —NHSO₂(1-4C)alkyl, —SO₂NH(1-4C)alkyl and —COPh (wherein the phenyl group is itself optionally substituted by a substituent selected from halo, (1-4C)alkyl, (1-4C)alkoxy, halo(1-4C)alkyl, halo(1-4C)alkoxy, (3-6C)cycloalkyl, (3-6C)cycloalkoxy, -(1-4)alkyl(3-6C)cycloalkyl, -(1-4C)alkoxy(3-6C)cycloalkyl, —S(O)p(1-4C)alkyl and —OSO₂(1-4C)alkyl.

Further suitable optional substituents for phenyl and aryl groups (including where the aryl group is attached to another group such as in aryl(1-4C)alkyl) are 1, 2 or 3 substituents independently selected from fluoro, chloro, cyano, nitro, amino, methylamino, dimethylamino, hydroxy, methyl, ethyl, methoxy, trifluoromethyl, trifluoromethoxy, methylcarbonyloxy, methoxycarbonyl, phenylcarbonyl, methylcarbonylamino, methylthio, methylsulfinyl and methylsulfonyl.

A suitable value for heteroaryl as a substituent on an aryl group is thiadiazolyl.

Further suitable optional substituents for phenyl and aryl groups (including where the aryl group is attached to another group such as in aryl(1-4C)alkyl) are 1, 2 or 3 substituents independently selected from fluoro, chloro, cyano, nitro, amino, methylamino, dimethylamino, hydroxy, methyl, ethyl, methoxy, trifluoromethyl, trifluoromethoxy, methylthio, methylsulfinyl and methylsulfonyl. A particular substituent is fluoro.

A heteroaryl group is an optionally substituted aromatic, monocyclic ring containing 5 to 7 atoms of which 1, 2, 3 or 4 ring atoms are chosen from nitrogen, sulphur or oxygen. Examples of heteroaryl are oxazolyl, oxadiazolyl, pyridyl, pyrimidinyl, imidazolyl, triazolyl, tetrazolyl, pyrazinyl, pyridazinyl, pyrrolyl, thienyl and furyl. Further examples of heteroaryl are thiadiazolyl and thiazolyl.

Suitable values for heteroaryl(1-4C)alkyl include any of the above examples of heteroaryl attached to a (1-4C)alkylchain, for example pyridylmethyl.

Suitable optional substituents for heteroaryl groups, unless otherwise defined, are 1, 2 or 3 substituents independently selected from halo, cyano, nitro, amino, hydroxy, (1-4C)alkyl (optionally substituted with 1, 2, 3, 4 or 5 halo), (1-4C)alkoxy (optionally substituted with 1, 2, 3, 4 or 5 halo), —S(O)p(1-4C)alkyl (wherein p is 0, 1 or 2), (1-4C)alkylamino and di-(1-4C)alkylamino. Further suitable optional substituents for heteroaryl groups are 1, 2 or 3 substituents independently selected from fluoro, chloro, cyano, nitro, amino, methylamino, dimethylamino, hydroxy, methyl, ethyl, methoxy, trifluoromethyl, trifluoromethoxy, methylthio, methylsulfinyl and methylsulfonyl.

If not stated elsewhere, suitable optional substituents for a particular group are those as stated for similar groups herein.

A compound of formula (I) may form stable acid or basic salts, and in such cases administration of a compound as a salt may be appropriate, and pharmaceutically acceptable salts may be made by conventional methods such as those described following.

Suitable pharmaceutically-acceptable salts include acid addition salts such as methanesulfonate, tosylate, α-glycerophosphate, fumarate, hydrochloride, citrate, maleate, tartrate and (less preferably) hydrobromide. Also suitable are salts formed with phosphoric and sulfuric acid. In another aspect suitable salts are base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine, tris-(2-hydroxyethyl)amine, N-methyl d-glucamine and amino acids such as lysine. There may be more than one cation or anion depending on the number of charged functions and the valency of the cations or anions. A preferred pharmaceutically-acceptable salt is the sodium salt.

However, to facilitate isolation of the salt during preparation, salts which are less soluble in the chosen solvent may be preferred whether pharmaceutically-acceptable or not.

Within the present invention it is to be understood that a compound of the formula (I) or a salt thereof may exhibit the phenomenon of tautomerism and that the formulae drawings within this specification can represent only one of the possible tautomeric forms. It is to be understood that the invention encompasses any tautomeric form which inhibits DPP-IV activity and is not to be limited merely to any one tautomeric form utilised within the formulae drawings.

It will be appreciated by those skilled in the art that certain compounds of formula (I) contain asymmetrically substituted carbon and/or sulphur atoms, and accordingly may exist in, and be isolated in, optically-active and racemic forms. Some compounds may exhibit polymorphism. It is to be understood that the present invention encompasses any racemic, optically-active, polymorphic or stereoisomeric form, or mixtures thereof, which form possesses properties useful in the inhibition of DPP-IV activity, it being well known in the art how to prepare optically-active forms (for example, by resolution of the racemic form by recrystallization techniques, by synthesis from optically-active starting materials, by chiral synthesis, by enzymatic resolution, by biotransformation, or by chromatographic separation using a chiral stationary phase) and how to determine efficacy for the inhibition of DPP-IV activity by the standard tests described hereinafter.

It is also to be understood that certain compounds of the formula (I) and salts thereof can exist in solvated as well as unsolvated forms such as, for example, hydrated forms. It is to be understood that the invention encompasses all such solvated forms which inhibit DPP-IV activity.

As stated before, we have discovered a range of compounds that have good DPP-IV inhibitory activity. They have good physical and/or pharmacokinetic properties in general. The following compounds possess preferred pharmaceutical and/or physical and/or pharmacokinetic properties.

Particular aspects of the invention comprise a compound of formula (I), or a pharmaceutically-acceptable salt thereof, wherein the substituents Ar, R¹ to R⁹ and other substituents mentioned above have values defined hereinbefore, or any of the following values (which may be used where appropriate with any of the definitions and embodiments disclosed hereinbefore or hereinafter):

In one embodiment of the invention are provided compounds of formula (I), in an alternative embodiment are provided pharmaceutically-acceptable salts of compounds of formula (I).

Particular values of variable groups are as follows. Such values may be used where appropriate with any of the other values, definitions, claims or embodiments defined hereinbefore or hereinafter.
1) Ar is unsubstituted phenyl
2) Ar is phenyl substituted with 1 group R⁹
3) Ar is phenyl substituted with 2 groups independently selected from R⁹
4) Ar is phenyl substituted with 3 groups independently selected from R⁹
5) R⁹ is halo, preferably fluoro
6) R⁹ is (1-2C)alkyl (optionally substituted with 1, 2, 3, 4or 5 halo), such as methyl, fluoromethyl, difluoromethyl or trifluoromethyl
7) R⁹ is methoxy
8) R⁹ is fluoromethoxy
9) R⁹ is difluoromethxoy or trifluoromethoxy
10) R⁹is cyano
11) Ar is fluorophenyl
12) Ar is difluorophenyl
13) Ar is trifluorophenyl
14) R⁵ is hydrogen or methyl
15) R⁵ is hydrogen
16) R⁶ is hydrogen or methyl
17) R⁶ is hydrogen
18) R⁵ or R⁶ is hydroxy
19) R⁵ and R⁶ together with the carbon to which they are attach form a cyclopropyl ring
20) R⁷ is hydrogen or methyl
21) R⁷ is hydrogen
22) R⁸ is hydrogen or methyl
23) R⁸ is hydrogen
24) R⁷ or R⁸ is hydroxy
25) R⁷ and R⁸ together with the carbon to which they are attach form a cyclopropyl ring
26) R⁴ is hydrogen
27) R⁴ is (1-4C)alkyl, for example methyl or ethyl, more particularly methyl
28) R⁴ is (1-4C)alkyl, substituted with hydroxy, for example hydroxymethyl
29) R⁴ is (1-4C)alkyl, substituted with (1-4C)alkoxy, for example methoxymethyl
30) R⁴ is (3-4C)cycloalkyl,
31) R⁴ is cyclopropyl
32) R⁴ is (1-4C)alkyl substituted by (3-4C)cycloalkyl,
33) R⁴ is cyclopropylmethyl 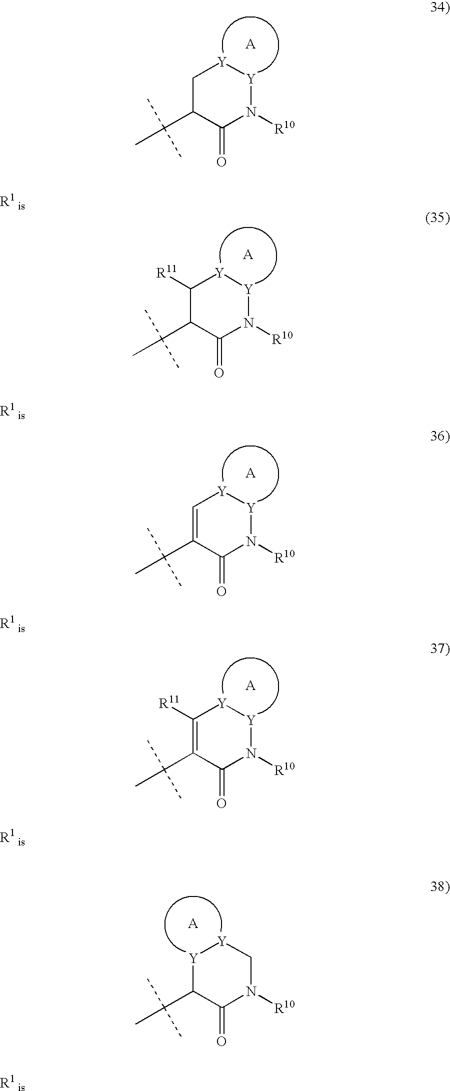
39) A is phenylene
40) A is a 5-membered heteroarylene ring
41) A is a 6-membered heteroarylene ring
42) A is pyridylene
43) one Y is carbon and the other is nitrogen
44) both Y are carbon
45) R¹⁰ is hydrogen
46) R¹⁰ is (1-4C)alkyl
47) R¹⁰ is hydroxy(1-4C)alkyl or (1-4C)alkoxy(1-4C)alkyl
48) R¹⁰ is (1-4C)alkylS(O)p(1-4C)alkyl
49) R¹⁰ is aryl(1-4C)alkyl or heteroaryl(1-4C)alkyl
50) R¹⁰ is aryl(1-4C)alkyl, particularly benzyl (optionally substituted)
51) R¹⁰ is benzyl optionally substituted with 1 or 2 substituents independently selected from methyl, fluoromethylsulfonyl, trifluoromethoxy, methoxy, methylcarbonyloxy, methoxycarbonyl, chloro, acetamido and nitro
52) R¹⁰ is benzyl optionally substituted with heteroaryl (particularly thiadiazolyl) or phenylcarbonyl
53) R¹⁰ is selected from -(1-4C)alkylCONH₂, -(1-4C)alkylCONH(1-4C)alkyl, -(1-4C)alkylCONdi(1-4C)alkyl, and -(2-4C)alkylNHCO(1-4C)alkyl
54) R¹⁰ is selected from -(2-4C)alkylNHSO₂(1-4C)alkyl, -(1-4C)alkylSO₂NH₂, -(1-4C)alkylSO₂NH(1-4C)alkyl and -(1-4C)alkylSO₂Ndi(1-4C)alkyl
55) R¹⁰ is selected from -(1-4C)alkylCO₂H, and -(1-4C)alkylCO₂(1-4C)alkyl
56) R¹⁰ is selected from benzyl (optionally substituted with 1 or 2 substituents independently selected from methyl, fluoromethylsulfonyl, trifluoromethoxy, methoxy, methylcarbonyloxy, methoxycarbonyl, chloro, acetamido, thiadiazolyl, phenylcarbonyl and nitro), methyl, ethyl, cyclopropylmethyl, methoxycarbonylmethyl and methoxy
56A) R¹⁰ is selected from optionally substituted benzyl, (1-4C)alkyl, (3-6C)cycloalkyl(1-4C)alkyl, -(1-4C)alkylCO₂(1-4C)alkyl and (1-4C)alkoxy
57) R² is phenyl or heteroaryl
58) R² is halo(1-6C)alkyl, dihalo(1-6C)alkyl, trifluoromethyl or pentafluoroethyl
59) R² is trifluoromethyl or pentafluoroethyl
60) R² is trifluoromethyl
61) R² is halo(1-6C)alkoxy or (1-4C)alkoxy
62) R² is cyano, halo, (1-4C)alkyl, or hydroxy
63) R² is methoxy
64) R² is amino, (1-4C)alkylamino or di(1-4C)alkylamino
65) R² is —CONH₂, —CONH(1-6C)alkyl, —CONdi(1-6C)alkyl, —NHCO(1-6C)alkyl, —S(O)₂NH₂, —SO₂NH(1-6C)alkyl, —SO₂Ndi(1-6C)alkyl or —NHSO₂(1-6C)alkyl
66) R² is —SO₂(1-6C)alkyl, —CO(1-6C)alkyl, —CO₂(1-6C)alkyl or —OCO(1-6C)alkyl
67) R² is (3-5C)cycloalkyl, for example cyclopropyl
68) R² is -(1-4C)alkyl(3-5C)cycloalkyl, for example cyclopropylmethyl
69) R² is halo(3-5C)cycloalkoxy (for example fluoro(3-5C)cycloalkoxy) or (3-5C)cycloalkoxy
70) R² is halo(3-5C)cycloalkyl, dihalo(3-5C)cycloalkyl or trihalo(3-5C)cycloalkyl
71) R² is fluoro(3-5C)cycloalkyl, difluoro(3-5C)cycloalkyl or trifluoro(3-5C)cycloalkyl
72) R² is (3-5C)cycloalkylamino
73) R² is selected from —CONH(3-5C)cycloalkyl, —NHCO(3-5C)cycloalkyl, —SO₂NH(3-5C)cycloalkyl and —NHSO₂(3-5C)cycloalkyl
74) R is selected from —SO₂(3-5C)cycloalkyl, —CO(3-5C)cycloalkyl, —CO₂(3-5C)cycloalkyl and —OCO(3-5C)cycloalkyl
75) A is unsubstituted
76) A is substituted by 1 R²
77) A is substituted by 2 R² (each independently selected from any value of R² hereinbefore or hereinafter)
78) R¹¹ is hydrogen
79) R¹¹ is phenyl, optionally substituted with fluoro
80) R¹¹ is selected from halo(1-4C)alkyl and halo(1-4C)alkoxy
81) R¹¹ is selected from (3-6C)cycloalkyl, (3-6C)cycloalkoxy, (3-6C)cycloalkyl(1-4C)alkyl and (3-6C)cycloalkyl(1-4C)alkoxy

In one aspect of the invention is provided a compound of the formula (I) as hereinbefore defined or a pharmaceutically acceptable salt thereof wherein
Ar is phenyl optionally substituted with 1, 2 or 3 groups independently selected from R⁹;
R⁹ is selected from halo, methyl, methoxy and trifluoromethyl;
R⁴, R⁵, R⁶, R⁷ and R⁸ are hydrogen;
is hydrogen;
is hydrogen;
is hydrogen;
R¹is 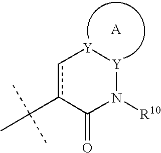
(wherein [symbol] is a single or double bond);
A is phenylene;
R¹⁰ is hydrogen, methyl or p-fluorobenzyl; and
R² is methoxy or trifluoromethyl.

In one aspect of the invention is provided a compound of the formula (I) as hereinbefore defined or a pharmaceutically acceptable salt thereof wherein
Ar is phenyl optionally substituted with 1, 2 or 3 groups independently selected from R⁹;
R⁹ is selected from halo, methyl, methoxy and trifluoromethyl;
R⁴, R⁵, R⁶, R⁷ and R⁸ are hydrogen;
R¹ is 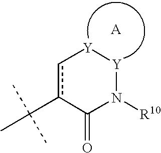
(wherein [symbol] is a single or double bond);
A is phenylene;
R¹⁰ is selected from hydrogen, optionally substituted benzyl, (1-4C)alkyl, (1-4C)alkyl, (3-6C)cycloalkyl(1-4C)alkyl, -(1-4C)alkylCO₂(1-4C)alkyl and (1-4C)alkoxy; and
R² is methoxy or trifluoromethyl.

In another aspect of the invention is provided a compound of the formula (I) as hereinbefore defined or a pharmaceutically acceptable salt thereof wherein
Ar is phenyl optionally substituted with 1, 2 or 3 groups independently selected from R⁹;
R⁹ is selected from halo, methyl, methoxy and trifluoromethyl;
R⁴, R⁵, R⁶, R⁷ and R⁸ are hydrogen;
R¹ is 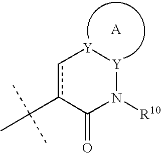
(wherein [symbol] is a single or double bond);
A is phenylene substituted with R²;
R¹⁰ is hydrogen, methyl or p-fluorobenzyl; and
R² is methoxy or trifluoromethyl.

In another aspect of the invention is provided a compound of the formula (I) as hereinbefore defined or a pharmaceutically acceptable salt thereof wherein
Ar is phenyl optionally substituted with 1, 2 or 3 groups independently selected from R⁹;
R⁹ is selected from halo, methyl, methoxy and trifluoromethyl;
R⁴, R⁵, R⁶, R⁷ and R⁸ are hydrogen;
R¹ is 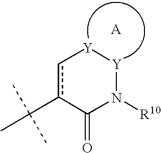
(wherein [symbol] is a single or double bond);
A is phenylene substituted with R²;
R¹⁰ is selected from hydrogen, optionally substituted benzyl, (1-4C)alkyl, (1-4C)alkyl, (3-6C)cycloalkyl(1-4C)alkyl, -(1-4C)alkylCO₂(1-4C)alkyl and (1-4C)alkoxy; and
R² is methoxy or trifluoromethyl.

In another aspect of the invention is provided a compound of the formula (I) as hereinbefore defined or a pharmaceutically acceptable salt thereof wherein
Ar is phenyl optionally substituted with 1, 2 or 3 groups independently selected from R⁹;
R⁹ is selected from halo, methyl, methoxy and trifluoromethyl;
R⁴, R⁵, R⁶, R⁷ and R⁸ are hydrogen;
R¹ is 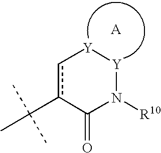
(wherein [symbol] is a single or double bond);
A is a 5-membered heteroarylene ring, optionally substituted by R²;
R¹⁰ is hydrogen, methyl or p-fluorobenzyl; and
R² is methoxy or trifluoromethyl.

In another aspect of the invention is provided a compound of the formula (I) as hereinbefore defined or a pharmaceutically acceptable salt thereof wherein
Ar is phenyl optionally substituted with 1, 2 or 3 groups independently selected from R⁹;
R⁹ is selected from halo, methyl, methoxy and trifluoromethyl;
R⁴, R⁵, R⁶, R⁷ and R⁸ are hydrogen;
R¹ is 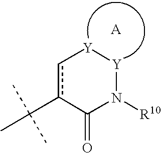
(wherein [symbol] is a single or double bond);
A is a 6-membered heteroarylene ring, optionally substituted by R²;
R¹⁰ is hydrogen, methyl or p-fluorobenzyl; and
R² is methoxy or trifluoromethyl.

In another aspect of the invention is provided a compound of the formula (I) as hereinbefore defined or a pharmaceutically acceptable salt thereof wherein
Ar is phenyl optionally substituted with 1, 2 or 3 groups independently selected from R⁹;
R⁹ is selected from halo, methyl, methoxy and trifluoromethyl;
R⁴, R⁵, R⁶, R⁷ and R⁸ are hydrogen;
R¹ is 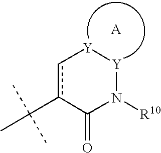
(wherein z,1 is a single or double bond);
A is a 6-membered heteroarylene ring, optionally substituted by R²;
R¹⁰ is selected from hydrogen, optionally substituted benzyl, (1-4C)alkyl, (1-4C)alkyl, (3-6C)cycloalkyl(1-4C)alkyl, -(1-4C)alkylCO₂(1-4C)alkyl and (1-4C)alkoxy; and
R² is methoxy or trifluoromethyl.

In another aspect of the invention is provided a compound of the formula (I) as hereinbefore defined or a pharmaceutically acceptable salt thereof wherein
Ar is phenyl optionally substituted with 1, 2 or 3 groups independently selected from R⁹;
R⁹ is selected from halo, methyl, methoxy and trifluoromethyl;
R⁴, R⁵, R⁶, R⁷ and R⁸ are hydrogen;
R¹ is 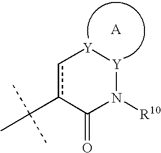
(wherein [symbol] is a single or double bond);
A is pyridylene, optionally substituted by R²;
R¹⁰ is hydrogen, methyl or p-fluorobenzyl; and
R² is methoxy or trifluoromethyl.

In another aspect of the invention is provided a compound of the formula (I) as hereinbefore defined or a pharmaceutically acceptable salt thereof wherein
Ar is phenyl optionally substituted with 1, 2 or 3 groups independently selected from R⁹;
R⁹ is selected from halo, methyl, methoxy and trifluoromethyl;
R⁴, R⁵, R⁶, R⁷ and R⁸ are hydrogen;
R¹ is 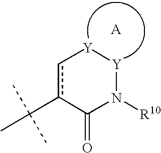
(wherein [symbol] is a single or double bond);
A is pyridylene, optionally substituted by R²;
R¹⁰ is selected from hydrogen, optionally substituted benzyl, (1-4C)alkyl, (1-4C)alkyl, (3-6C)cycloalkyl(1-4C)alkyl, -(1-4C)alkylCO₂(1-4C)alkyl and (1-4C)alkoxy; and
R² is methoxy or trifluoromethyl.

In another aspect of the invention is provided a compound of the formula (I) as hereinbefore defined or a pharmaceutically acceptable salt thereof wherein
Ar is phenyl optionally substituted with 1, 2 or 3 fluoro substituents;
R⁴, R⁵, R⁶, R⁷ and R⁸ are hydrogen;
R¹ is 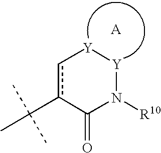
(wherein [symbol] is a single or double bond);
A is phenylene, optionally substituted by R²;
R¹⁰ is hydrogen, methyl or p-fluorobenzyl; and
R² is methoxy or trifluoromethyl.

In another aspect of the invention is provided a compound of the formula (I) as hereinbefore defined or a pharmaceutically acceptable salt thereof wherein
Ar is phenyl optionally substituted with 1, 2 or 3 fluoro substituents;
R⁴, R⁵, R⁶, R⁷ and R⁸ are hydrogen;
R¹ is 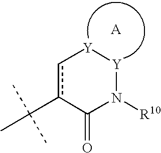
(wherein [symbol] is a single or double bond);
A is phenylene, optionally substituted by R²;
R¹⁰ is selected from hydrogen, optionally substituted benzyl, (1-4C)alkyl, (1-4C)alkyl, (3-6C)cycloalkyl(1-4C)alkyl, -(1-4C)alkylCO₂(1-4C)alkyl and (1-4C)alkoxy; and
R² is methoxy or trifluoromethyl.

In one aspect of the invention is provided a compound of the formula (I) as hereinbefore defined or a pharmaceutically acceptable salt thereof wherein
Ar is phenyl optionally substituted with 1, 2 or 3 fluoro substituents;
R⁴, R⁵, R⁶, R⁷ and R⁸ are hydrogen;
R¹ is 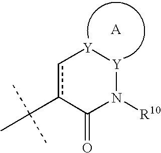
(wherein [symbol] is a single or double bond);
A is a 5-membered heteroarylene ring, optionally substituted by R²;
R¹⁰ is hydrogen, methyl or p-fluorobenzyl; and
R² is methoxy or trifluoromethyl.

In one aspect of the invention is provided a compound of the formula (I) as hereinbefore defined or a pharmaceutically acceptable salt thereof wherein
Ar is phenyl optionally substituted with 1, 2 or 3 fluoro substituents;
R⁴, R⁵, R⁶, R⁷ and R⁸ are hydrogen;
R¹ is 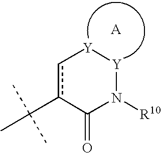
(wherein [symbol] is a single or double bond);
A is a 6-membered heteroarylene ring, optionally substituted by R²;
R¹⁰ is hydrogen, methyl or p-fluorobenzyl; and
R² is methoxy or trifluoromethyl.

In one aspect of the invention is provided a compound of the formula (I) as hereinbefore defined or a pharmaceutically acceptable salt thereof wherein
Ar is phenyl optionally substituted with 1, 2 or 3 fluoro substituents;
R⁴, R⁵, R⁶, R⁷ and R⁸ are hydrogen;
R¹ is 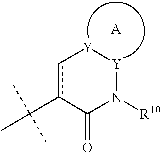
(wherein [symbol] is a single or double bond);
A is a 6-membered heteroarylene ring, optionally substituted by R²;
R¹⁰ is selected from hydrogen, optionally substituted benzyl, (1-4C)alkyl, (1-4C)alkyl, (3-6C)cycloalkyl(1-4C)alkyl, -(1-4C)alkylCO₂(1-4C)alkyl and (1-4C)alkoxy; and
R² is methoxy or trifluoromethyl.

In another aspect of the invention is provided a compound of the formula (I) as hereinbefore defined or a pharmaceutically acceptable salt thereof wherein
Ar is phenyl optionally substituted with 1, 2 or 3 fluoro substituents;
R⁴, R⁵, R⁶, R⁷ and R⁸ are hydrogen;
R¹ is 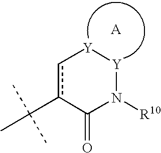
(wherein [symbol] is a single or double bond);
A is pyridylene, optionally substituted by R²;
R¹⁰ is hydrogen, methyl or p-fluorobenzyl; and
R² is methoxy or trifluoromethyl.

In another aspect of the invention is provided a compound of the formula (I) as hereinbefore defined or a pharmaceutically acceptable salt thereof wherein
Ar is phenyl optionally substituted with 1, 2 or 3 fluoro substituents;
R⁴, R⁵, R⁶, R⁷ and R⁸ are hydrogen;
R¹ is 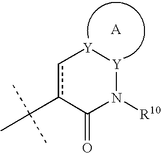
(wherein [symbol] is a single or double bond);
A is pyridylene, optionally substituted by R²;
R¹⁰ is selected from hydrogen, optionally substituted benzyl, (1-4C)alkyl, (1-4C)alkyl, (3-6C)cycloalkyl(1-4C)alkyl, -(1-4C)alkylCO₂(1-4C)alkyl and (1-4C)alkoxy; and
R² is methoxy or trifluoromethyl.

In another aspect of the invention is provided a compound of the formula (I) as hereinbefore defined or a pharmaceutically acceptable salt thereof wherein
Ar is phenyl optionally substituted with 1, 2 or 3 fluoro substituents;
R⁴, R⁵, R⁶, R⁷ and R⁸ are hydrogen;
R¹ is 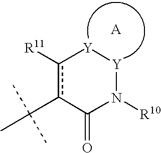
(wherein [symbol] is a single or double bond);
A is pyridylene, optionally substituted by R²;
R¹¹ is phenyl, optionally substituted with fluoro;
R¹⁰ is selected from hydrogen, optionally substituted benzyl, (1-4C)alkyl, (1-4C)alkyl, (3-6C)cycloalkyl(1-4C)alkyl, -(1-4C)alkylCO₂(1-4C)alkyl and (1-4C)alkoxy; and
R² is methoxy or trifluoromethyl.

In one aspect of the invention is provided a compound of the formula (I) as hereinbefore defined or a pharmaceutically acceptable salt thereof wherein
Ar is phenyl optionally substituted with 1, 2 or 3 groups independently selected from R⁹;
R⁹ is selected from halo, methyl, methoxy and trifluoromethyl;
R⁴, R⁵, R⁶, R⁷ and R⁸ are hydrogen;
R¹ is 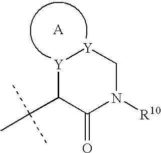
A is phenylene, optionally substituted by R²;
R¹⁰ is hydrogen, methyl or p-fluorobenzyl; and
R² is methoxy or trifluoromethyl.

In another aspect of the invention is provided a compound of the formula (I) as hereinbefore defined or a pharmaceutically acceptable salt thereof wherein
Ar is phenyl optionally substituted with 1, 2 or 3 groups independently selected from R⁹;
R⁹is selected from halo, methyl, methoxy and trifluoromethyl;
R⁴, R⁵, R⁶, R⁷ and R⁸ are hydrogen;
R¹ is 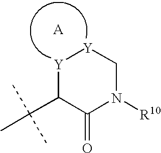
A is phenylene substituted with R²;
R¹⁰ is hydrogen, methyl or p-fluorobenzyl; and
R² is methoxy or trifluoromethyl.

In another aspect of the invention is provided a compound of the formula (I) as hereinbefore defined or a pharmaceutically acceptable salt thereof wherein
Ar is phenyl optionally substituted with 1, 2 or 3 groups independently selected from R⁹;
R⁹is selected from halo, methyl, methoxy and trifluoromethyl;
R⁴, R⁵, R⁶, R⁷ and R⁸ are hydrogen;
R¹ is 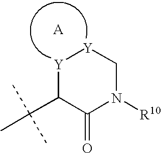
A is a 5-membered heteroarylene ring, optionally substituted by R²;
R¹⁰ is hydrogen, methyl or p-fluorobenzyl; and
R² is methoxy or trifluoromethyl.

In another aspect of the invention is provided a compound of the formula (I) as hereinbefore defined or a pharmaceutically acceptable salt thereof wherein
Ar is phenyl optionally substituted with 1, 2 or 3 groups independently selected from R⁹;
R⁹is selected from halo, methyl, methoxy and trifluoromethyl;
R⁴, R⁵, R⁶, R⁷ and R⁸ are hydrogen;
R¹ is 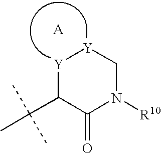
A is a 6-membered heteroarylene ring, optionally substituted by R²;
R¹⁰ is hydrogen, methyl or p-fluorobenzyl; and
R² is methoxy or trifluoromethyl.

In another aspect of the invention is provided a compound of the formula (I) as hereinbefore defined or a pharmaceutically acceptable salt thereof wherein
Ar is phenyl optionally substituted with 1, 2 or 3 groups independently selected from R⁹;
R⁹ is selected from halo, methyl, methoxy and trifluoromethyl;
R⁴, R⁵, R⁶, R⁷ and R⁸ are hydrogen;
R¹ is 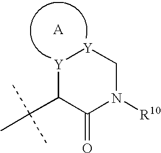
A is pyridylene, optionally substituted by R²;
R¹⁰ is hydrogen, methyl or p-fluorobenzyl; and
R² is methoxy or trifluoromethyl.

In another aspect of the invention is provided a compound of the formula (I) as hereinbefore defined or a pharmaceutically acceptable salt thereof wherein
Ar is phenyl optionally substituted with 1, 2 or 3 fluoro substituents;
R⁴, R⁵, R⁶, R⁷ and R⁸ are hydrogen;
R¹ is 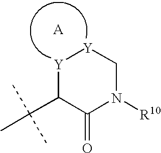
A is phenylene, optionally substituted by R²;
R¹⁰ is hydrogen, methyl or p-fluorobenzyl; and
R² is methoxy or trifluoromethyl.

In one aspect of the invention is provided a compound of the formula (I) as hereinbefore defined or a pharmaceutically acceptable salt thereof wherein
Ar is phenyl optionally substituted with 1, 2 or 3 fluoro substituents;
R⁴, R⁵, R⁶, R⁷ and R⁸ are hydrogen;
R¹ is 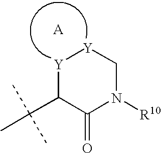
A is a 5-membered heteroarylene ring, optionally substituted by R²;
R¹⁰ is hydrogen, methyl or p-fluorobenzyl; and
R² is methoxy or trifluoromethyl.

In one aspect of the invention is provided a compound of the formula (I) as hereinbefore defined or a pharmaceutically acceptable salt thereof wherein
Ar is phenyl optionally substituted with 1, 2 or 3 fluoro substituents;
R⁴, R⁵, R⁶, R⁷ and R⁸ are hydrogen;
R¹ is 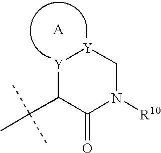
A is a 6-membered heteroarylene ring, optionally substituted by R²;
R¹⁰ is hydrogen, methyl or p-fluorobenzyl; and
R² is methoxy or trifluoromethyl.

In one aspect of the invention is provided a compound of the formula (I) as hereinbefore defined or a pharmaceutically acceptable salt thereof wherein
Ar is phenyl optionally substituted with 1, 2 or 3 fluoro substituents;
R⁴, R⁵, R⁶, R⁷ and R⁸ are hydrogen;
R¹ is 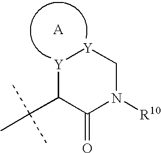
A is pyridylene, optionally substituted by R²;
R¹⁰ is hydrogen, methyl or p-fluorobenzyl; and
R² is methoxy or trifluoromethyl.

Particular compounds of the invention are of the formula (Ia): 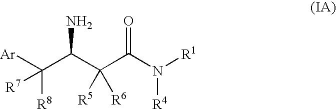
wherein Ar, R¹ to R¹¹ are as defined in any one of the definitions, embodiments or aspects contained herein before or hereinafter.

Further preferred compounds of the invention are each of the Examples, each of which provides a further independent aspect of the invention. In further aspects, the present invention also comprises any two or more compounds of the Examples.

In a further aspect of the invention there is provided a compound selected from:
(3R)-3-amino-4-(2-fluorophenyl)-N-(2-oxo-1,2,3,4-tetrahydroquinolin-3-yl)butanamide;
(3R)-3-amino-4-(2-fluorophenyl)-N-(2-oxo-1,2,3,4-tetrahydro-1,5-naphthyridin-3-yl)butanamide;
(R)-3-amino-4-(2-fluorophenyl)-N-(2-oxo-1,2,3,4-tetrahydro-1,8-naphthyridin-3-yl)-butanamide dihydrochloride (and individual diasteroemers thereof);
(R)-3-amino-4-(4-fluorophenyl)-N-(2-oxo-1,2,3,4-tetrahydro-1,8-naphthyridin-3-yl)-butanamide;
(R)-3-amino-4-(2-fluorophenyl)-N-(2-oxo-1,2,3,4-tetrahydro-1,7-naphthyridin-3-yl)-butanamide;
(R)-3-amino-4-(4-fluorophenyl)-N-(2-oxo-1,2,3,4-tetrahydro-1,7-naphthyridin-3-yl)-butanamide;
(R)-3-amino-4-(2-fluorophenyl)-N-(2-oxo-1,2,3,4-tetrahydro-1,6-naphthyridin-3-yl)-butanamide;
(R)-3-amino-4-(4-fluorophenyl)-N-(2-oxo-1,2,3,4-tetrahydro-1,6-naphthyridin-3-yl)-butanamide;
(3R)-3-amino-4-(2-fluorophenyl)-N-(1-methyl-2-oxo-1,2,3,4-tetrahydro-1,5-naphthyridin-3-yl)butanamide dihydrochloride;
(3R)-3-amino-N-[1-(4-fluorobenzyl)-2-oxo-1,2,3,4-tetrahydro-1,5-naphthyridin-3-yl]-4-(2-fluorophenyl)butanamide dihydrochloride;
(3R)-3-amino-4-(2,5-difluorophenyl)-N-(2-oxo-1,2,3,4-tetrahydro-1,8-naphthyridin-3-yl)butanamide;
(3R)-3-amino-4-(2,5-difluorophenyl)-N-(2-oxo-1,2,3,4-tetrahydro-1,5-naphthyridin-3-yl)butanamide;
(3R)-3-amino-4-(2-fluorophenyl)-N-(2-oxo-1,2-dihydroquinolin-3-yl)butanamide hydrochloride;
(3R)-3-amino-4-(2-fluorophenyl)-N-(5-methoxy-2-oxo-1,2,3,4-tetrahydroquinolin-3-yl)butanamide hydrochloride;
(3R)-3-amino-4-(2,5-difluorophenyl)-N-[(3S)-2-oxo-1,2,3,4-tetrahydro-1,8-naphthyridin-3-yl]butanamide;
(3R)-3-amino-4-(2,5-difluorophenyl)-N-[(3R)-2-oxo-1,2,3,4-tetrahydro-1,8-naphthyridin-3-yl]butanamide dihydrogen chloride;
(3R)-3-amino-4-(2,4,5-trifluorophenyl)-N-[(3S)-2-oxo-1,2,3,4-tetrahydro-1,8-naphthyridin-3-yl]butanamide;
(3R)-3-amino-4-(2,4,5-trifluorophenyl)-N-[(3R)-2-oxo-1,2,3,4-tetrahydro-1,8-naphthyridin-3-yl]butanamide;
(3R)-3-amino-4-(2-fluorophenyl)-N-(1-methyl-2-oxo-1,2,3,4-tetrahydro-1,8-naphthyridin-3-yl)butanamide;
(3R)-3-amino-4-(2,5-difluorophenyl)-N-(2-oxo-1,2,3,4-tetrahydro-1,6-naphthyridin-3-yl)butanamide;
(3R)-3-amino-4-(2,5-difluorophenyl)-N-(2-oxo-1,2,3,4-tetrahydro-1,7-naphthyridin-3-yl)butanamide;
(3R)-3-amino-N-(1-ethyl-2-oxo-1,2,3,4-tetrahydro-1,8-naphthyridin-3-yl)-4-(2-fluorophenyl)butanamide;
(3R)-3-amino-N-[1-(cyclopropylmethyl)-2-oxo-1,2,3,4-tetrahydro-1,8-naphthyridin-3-yl]-4-(2-fluorophenyl)butanamide;
(3R)-3-amino-4-(2,5-difluorophenyl)-N-[1-(4-fluorobenzyl)-2-oxo-1,2,3,4-tetrahydro-1,8-naphthyridin-3-yl]butanamide;
(3S)-3-amino-3-(2,5-difluorophenyl)-N-[1-(4-fluorobenzyl)-2-oxo-1,2,3,4-tetrahydro-1,8-naphthyridin-3-yl]-N-methylpropanamide;
methyl [3-{[(3R)-3-amino-4-(2-fluorophenyl)butanoyl]amino}-2-oxo-3,4-dihydroquinolin-1(2H)-yl]acetate;
(3R)-3-amino-4-(2-fluorophenyl)-N-{1-[4-(methylsulfonyl)benzyl]-2-oxo-1,2,3,4-tetrahydro-1,5-naphthyridin-3-yl}butanamide dihydrochloride;
(3R)-3-amino-4-(2-fluorophenyl)-N-{2-oxo-1-[4-(trifluoromethoxy)benzyl]-1,2,3,4-tetrahydro-1,5-naphthyridin-3-yl}butanamide dihydrochloride;
(3R)-3-amino-4-(2-fluorophenyl)-N-{2-oxo-1-[4-(trifluoromethyl)benzyl]-1,2,3,4-tetrahydro-1,5-naphthyridin-3-yl}butanamide dihydrochloride;
(3R)-3-amino-4-(2-fluorophenyl)-N-{2-oxo-1-[4-(1,2,3-thiadiazol-4-yl)benzyl]-1,2,3,4-tetrahydro-1,5-naphthyridin-3-yl}butanamide dihydrochloride;
(3R)-3-amino-4-(2-fluorophenyl)-N-{1-[2-(4-methoxyphenyl)ethyl]-2-oxo-1,2,3,4-tetrahydro-1,5-naphthyridin-3-yl}butanamide dihydrochloride;
(3R)-3-amino-4-(2-fluorophenyl)-N-{1-[2-(4-fluorophenyl)ethyl]-2-oxo-1,2,3,4-tetrahydro-1,5-naphthyridin-3-yl}butanamide dihydrochloride;
methyl 4-{[3-{[(3R)-3-amino-4-(2-fluorophenyl)butanoyl]amino}-2-oxo-3,4-dihydro-1,8-naphthyridin-1(2H)-yl]methyl}benzoate;
(3R)-3-amino-N-[1-(3-chloro-4-methoxybenzyl)-2-oxo-1,2,3,4-tetrahydro-1,8-naphthyridin-3-yl]-4-(2-fluorophenyl)butanamide;
(3R)-3-amino-N-[1-(4-benzoylbenzyl)-2-oxo-1,2,3,4-tetrahydro-1,8-naphthyridin-3-yl]-4-(2-fluorophenyl)butanamide;
(3R)-N-{1-[4-(acetylamino)benzyl]-2-oxo-1,2,3,4-tetrahydro-1,8-naphthyridin-3-amino-4-(2-fluorophenyl)butanamide;
(3R)-3-amino-N-(6-fluoro-2-oxo-1,2,3,4-tetrahysroquinolin-3-yl)-4-(2-fluorophenyl)butanamide monohydrochloride;
(3R)-3-amino-N-(6-methoxy-2-oxo-1,2,3,4-tetrahysroquinolin-3-yl)-4-(2-fluorophenyl)butanamide;
(3R)-3-amino-4-(2-fluorophenyl)-N-(5-methyl-2-oxo-1,2,3,4-tetrahysroquinolin-3-yl)butanamide monohydrochloride;
(3R)-3-amino-4-(2,5-difluorophenyl)-N-(5-methoxy-2-oxo-1,2,3,4-tetrahysroquinolin-3-yl)butanamide;
(R)-3-amino-4-(2-fluorophenyl)-N-((3R,4S)-1-methoxy-2-oxo-4-phenyl-1,2,3,4-tetrahydroquinolin-3-yl)butanamide;
(R)-3-amino-4-(2-fluorophenyl)-N-((3S,4R)-1-methoxy-2-oxo-4-phenyl-1,2,3,4-tetrahydroquinolin-3-yl)butanamide; and
(3R)-3-amino-4-(2-fluorophenyl)-N-[4-(4-fluorophenyl)-2-oxo-1,2-dihydroquinolin-3-yl]butanamide hydrochloride;

or a pharmaceutically-acceptable salt thereof.

Process

A compound of formula (I) and its pharmaceutically-acceptable salts may be prepared by any process known to be applicable to the preparation of chemically related compounds. Such processes, when used to prepare a compound of the formula (I), or a pharmaceutically-acceptable salt thereof, are provided as a further feature of the invention.

In a further aspect the present invention also provides that the compounds of the formulae (I) and pharmaceutically-acceptable salts thereof, can be prepared by a process (a) to (c) as follows (wherein the variables are as defined hereinbefore or after unless otherwise stated):
a) Coupling a compound of the formula (II) wherein P is a protecting group 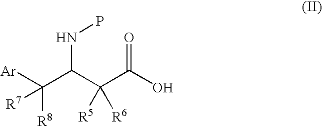
   with a compound of the formula (IIIa) or (IIIb); 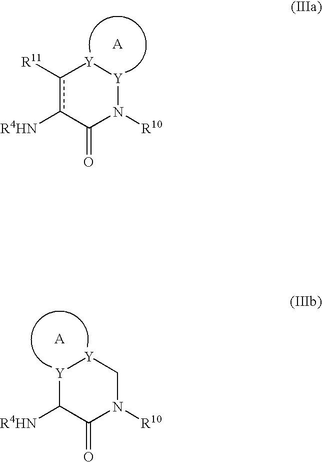
   to give a compound of the formula (IVa) or (IVb); 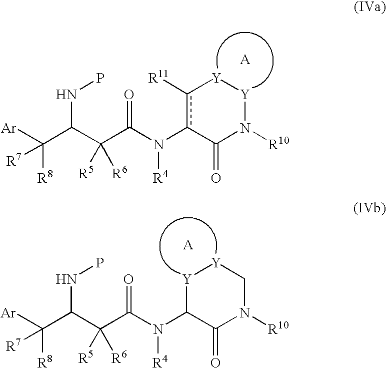
b) removing the protecting group P to give a compound of the formula (I);
c) optionally forming a pharmaceutically acceptable salt.

Compounds of the formula (II) are generally commercially available or may be made by processes known in the art for making β-amino acids, particularly the method of N. Ikemoto et al. J. Amer. Chem. Soc 2004, 126(10), 3048. Suitably the protecting group P is a carbamate protecting group such as a BOC group. Further suitable processes for making compounds of formula (II) may be found in International patent application WO 2004/032836 (see for example pages 31-32) and references therein.

Suitable coupling conditions for step a) are any of those known in the art for coupling together acids and bases for example standard peptide coupling reagents known in the art, or for example carbonyldiimidazole, 1-ethyl-3-(3-dimethylaminopropyl)carbodi-imide hydrochloride (EDCI) and dicyclohexyl-carbodiimide (DCCI), optionally in the presence of a catalyst such as 1-hydroxybenzotriazole, dimethylaminopyridine or 4-pyrrolidinopyridine, optionally in the presence of a base for example triethylamine, di-isopropylethylamine, pyridine, or 2,6-dialkylpyridines such as 2,6-lutidine or 2,6-di-tert-butylpyridine. Suitable solvents include dimethylacetamide, dichloromethane, benzene, tetrahydrofuran and dimethylformamide. The coupling reaction may conveniently be performed at a temperature in the range of −40 to 40° C.

Removal of the protecting group P may be achieved by any suitable method known in the art. Where P is a carbamate group such as a BOC group, hydrolysis of the BOC group may be achieved using aqueous acid, for example a solution of aqueous HCl in dioxan. Conditions suitable for removing the protecting group P, such as treatment with an acid such as HCl, may result information of a salt of a compound of the formula (I), which may optionally be treated to give the free base form or to give an alternative (pharmaceutically acceptable) salt form.

Compounds of the formula (IIIa) wherein A is phenylene and [symbol] is a single bond and R¹⁰ is hydrogen may be made from 3-amino-3,4-dihydroquinolin-2-(1H)-one hydrochloride (J. Med. Chem., 28, 1985, 1511-16). Compounds of the formula (IV) wherein A is phenylene and [symbol] is a double bond may be prepared by the reductive cyclisation of a compound of formula (V), using for example tin (II) chloride in hydrochloric acid, followed by removal of the Boc protecting group, using for example trifluoroacetic acid. Compounds of formula (V) may be prepared by reaction of compounds of formula (VI) by reaction with a compound of formula (VII) in the presence of a base, for example tetramethylguanidine. Compounds of formula (VI) are commercially available or described in the literature.

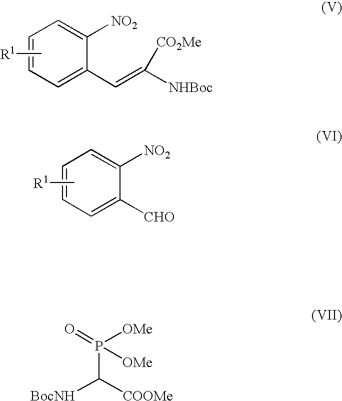

Compounds of the formula (IIIa) wherein A is heterocyclylene may be prepared from cyclisation of suitably functionalised heterocycles. For example, when A is a fused pyridine, 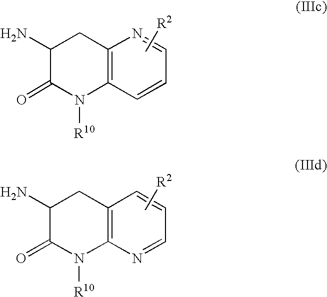
compounds of formula (IIIc) and (IIId) may be prepared from an appropriately substituted methylnitropyridine or aminopyridine according to Schemes 2 and 3:

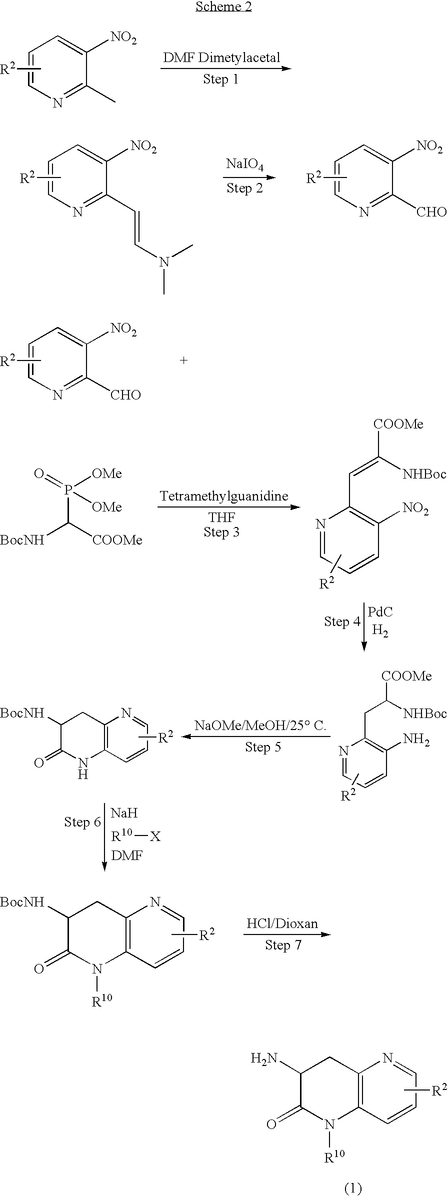

Steps 1 and 2 may be carried out by the process described in Tetrahedron 1998, 54(23), 6311-6318.

Step 3 may be carried out by the method described in Synthesis 1992 (5), 487. Assymetric hydrogenation reactions of olefins as shown in Step 4 are well known (see for example, J Am Chem Soc 1993, 115, 10125-10138) and lead to homochiral final products. Step 5 may alternatively be carried out by hydrolysing the ester and activating the resulting acid with a carbodiimide such as EDCI or DCCI, or by preparing an acid chloride, or activated ester such as an N-hydroxysuccinimide ester. Suitable bases are organic base such as triethylaamine or di-isopropylethylamine (DIPEA) or 1,8-diazabicyclo[5.4.0]undec-7-ene (DBU).

In Step 6 X is a leaving group, for example Cl, Br, I, OMesyl. In Step 7 alternative solvents such as dichloromethane or other acids such as trifluoroacetic acid can be used. 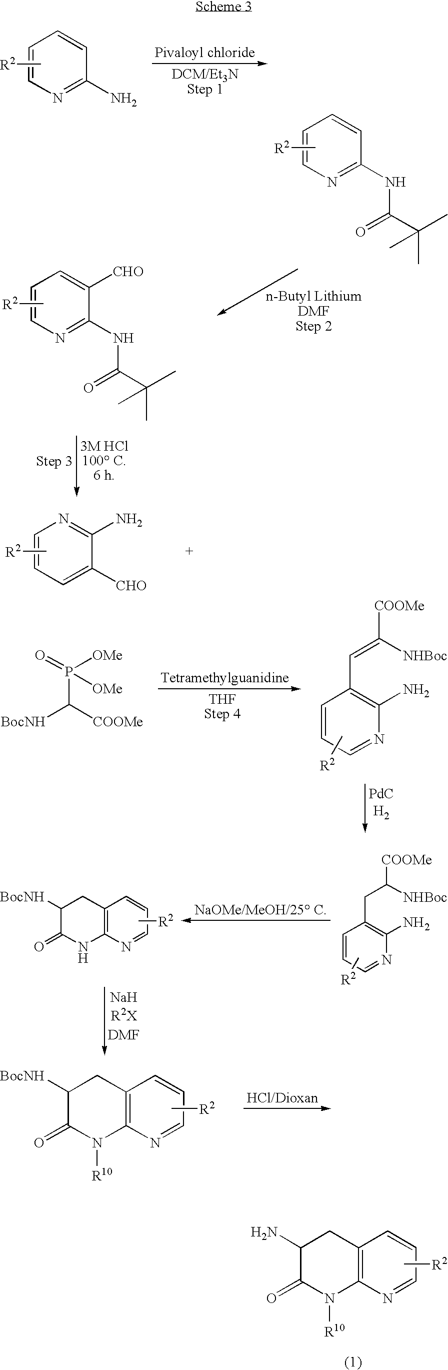
Steps 1, 2, 3 and 4 are described in J Org Chem 1983, 48, 3401-3408.

The processes described above and shown in Schemes 2 and 3 may also be applied to other isomeric pyridines or six membered heterocycles containing more than one nitrogen.

Routes to isomeric pyridines from nitropyridine derivatives are illustrated in the schemes below:

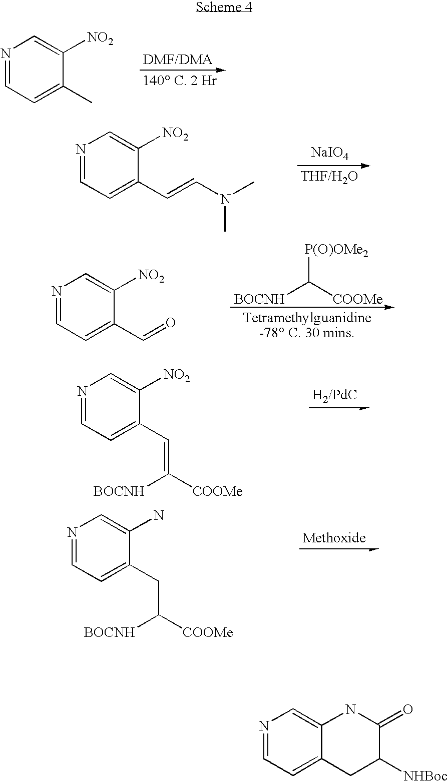

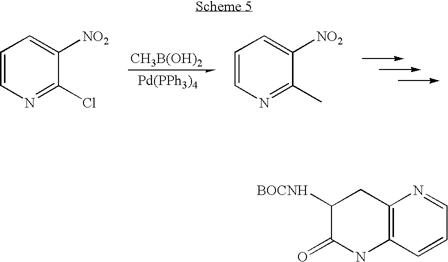

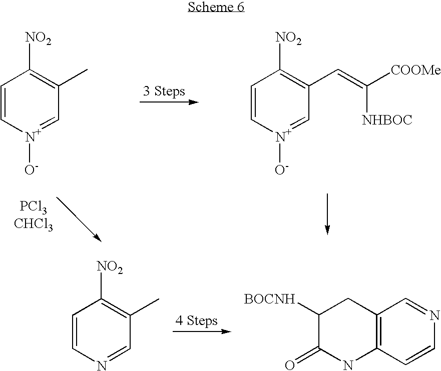

Compounds of the formula (IIIa) wherein A is a heteroarylene and there is a bridgehead nitrogen, for example a compound of formula (IIIe), 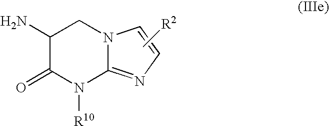
may be prepared by cyclisation of a compound of the formula (VIII): 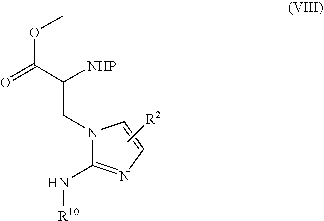
wherein P is an amino protecting group such as triphenylmethyl. This transformation is induced by heating compounds of the formula (VIII) to reflux in a solvent, for example, ethanol.

Compounds of the formula (VIII) may be prepared from a compound of the formula (IX) by hydrogenation using a catalyst such as Pd/C at ambient temperature, followed as appropriate by introduction of R¹⁰, for example by alkylation of the primary amino group.

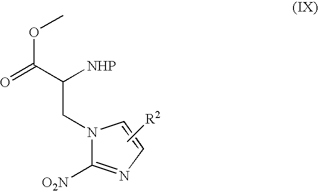

Compounds of the formula (IX) may be prepared from compounds of the formula (X) and (XI): 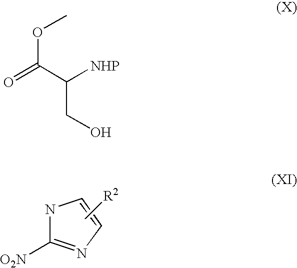
using conditions known for the Mitsunobu reaction (Bull. Chem. Soc. Jpn., 1967, 40, 23 80). Compounds of the formulae (X) and (XI) are commercially available.

Compounds of the formula (IIIa) wherein A is heteroarylene and there is a bridgehead heteroatom may be made by analogous chemistry to that shown for making compounds of the formula (IIIe).

Compounds of the formula (IIIb), such as compound (XII) below where A is phenylene and R⁴ is hydrogen, may be made according to the methodology of Ishai et al (Ishai, D. Ben; Sataty, I.; Peled, N.; Goldshare, R.; Tetrahedron; 43; 2; 1987; 439-450). Compounds of formula (IIIb) wherein A is heteroarylene may be made by analogous processes to those described above for compounds of formula (IIIa).

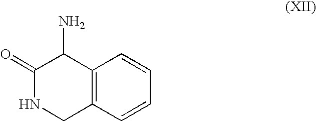

Compounds of the formula (IIIa) wherein z,1 is a single bond, R⁴ is H and R¹⁰ is an alkyl or functionalised alkyl group may be prepared by treating compounds of formula (IIIa) wherein R⁴ is tert-butoxycarbonyl and R¹⁰ is H with a base, for example sodium hydride, followed by an alkylating agent such as iodomethane or a functionalised alkylating agent such as 4-fluorobenzyl bromide.

Compounds of the formula (IIIa) wherein z,1 is a single bond, and both R⁴ and R¹⁰ are an alkyl or functionalised alkyl group may be prepared by treating compounds of formula (IIIa) wherein R⁴ is tert-butoxycarbonyl and R¹⁰ is H with 2 or more equivalents of a base such as sodium hydride followed by 2 or more equivalents of an alkylating agent or a functionalised alkylating agent.

Compounds of the formula (IIIa) wherein [symbol] is a single bond, R⁴ is alkyl and R¹⁰ is H may be prepared by using the procedures as described above to introduce a protecting group, such as 4-methoxybenzyl, as R¹⁰, then further alkylating at R⁴ followed by removal of the R¹⁰ protecting group, for example by catalytic hydrogenation.

Compounds of the formula (IIIa) wherein [symbol] is a double bond, R⁴ and R¹⁰ are H, and wherein R¹¹ is substituted phenyl may be prepared by hydrolysis, for example in a mixture of acetic and sulphuric acids, of the corresponding compounds wherein R⁴ is an acyl group, for example acetyl. Such compounds where R⁴ is acetyl can be prepared by cyclisation of a compound of formula (XIII) in the presence of a base such as potassium tert-butoxide.

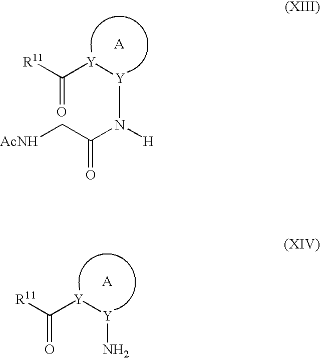

Compounds of formula (XIII) are prepared by reaction of N-acetyl glycine with an aminoketone of formula (XIV) in the presence of a coupling agent such as isobutyl chloroformate. Compounds of formula (XIV) are commercially available or readily prepared by standard methods.

Compounds of the formula (IIIa) wherein z,1 is a single bond, A is phenylene, R⁴ is H, R¹⁰ is OMe and R¹¹ is phenyl may be prepared from an N-methoxydiphenylalanine derivative of formula (XV) by oxidative cyclisation in the presence of, for example, bis(triflouroacetoxy)iodobenzene, followed by acidic deprotection. The chirality of the 3-position can be defined by utilising the appropriate (R) or (S) N-methoxydiphenylalanine derivative. The products of the cyclisation are predominantly trans-, resulting in the formation of either the (3R, 4S) or (3S, 4R) diastereoisomer. Cyclisation of compounds of formula (XV) which have substituents in the phenyl rings may lead to isomeric mixtures, which may in turn be separated, for example by chromatography.

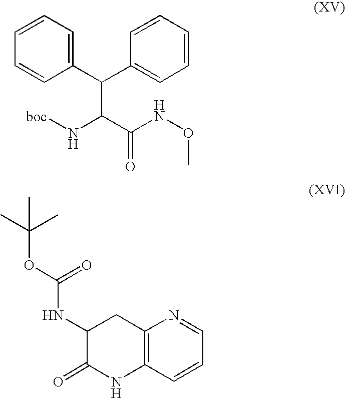

Compounds of formula (XV) may be prepared by condensation of the appropriately substituted diphenylalanine derivative with methoxylamine in the presence of a coupling agent, for example EDAC.

A compound of formula (XVI) which has the (S) stereochemistry at C-3 can be prepared by reaction of 3-amino-2-bromopyridine with the organozinc reagent obtained from reaction of methyl N-(tert-butoxycarbonyl)-3-iodo-L-alaninate with zinc metal in the presence of chlorotrimethylsilane. A compound of formula (XVI) which has the (R) stereochemistry at C-3 can be prepared similarly, starting from methyl N-(tert-butoxycarbonyl)-3-iodo-D-alaninate.

It will be appreciated that certain of the various ring substituents in the compounds of the present invention, for example R², may be introduced by standard aromatic substitution reactions or generated by conventional functional group modifications either prior to or immediately following the processes mentioned above, and as such are included in the process aspect of the invention. Such reactions may convert one compound of the formula (I) into another compound of the formula (I). Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogen group. Particular examples of modifications include the reduction of a nitro group to an amino group by for example, catalytic hydrogenation with a nickel catalyst or treatment with iron in the presence of hydrochloric acid with heating; oxidation of alkylthio to alkanesulphinyl or alkanesulphonyl.

If not commercially available, the necessary starting materials for the procedures such as those described above may be made by procedures which are selected from standard organic chemical techniques, techniques which are analogous to the synthesis of known, structurally similar compounds, techniques which are described or illustrated in the references given above, or techniques which are analogous to the above described procedure or the procedures described in the examples.

It is noted that many of the starting materials for synthetic methods as described above are commercially available and/or widely reported in the scientific literature, or could be made from commercially available compounds using adaptations of processes reported in the scientific literature. The reader is further referred to Advanced Organic Chemistry, 4^{th} Edition, by Jerry March, published by John Wiley & Sons 1992, for general guidance on reaction conditions and reagents.

It will be appreciated that some intermediates to compounds of the formula (I) are also novel and these are provided as separate independent aspects of the invention.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, John Wiley and Sons, 1991).

Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Thus, if reactants include, for example, groups such as amino, carboxy or hydroxy it may be desirable to protect the group in some of the reactions mentioned herein.

Examples of a suitable protecting group for a hydroxy group is, for example, an acyl group, for example an alkanoyl group such as acetyl, an aroyl group, for example benzoyl, a silyl group such as trimethylsilyl or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively a silyl group such as trimethylsilyl may be removed, for example, by fluoride or by aqueous acid; or an arylmethyl group such as a benzyl group may be removed, for example, by hydrogenation in the presence of a catalyst such as palladium-on-carbon.

A suitable protecting group for an amino group is, for example, an acyl group, for example an alkanoyl group such as acetyl, an alkoxycarbonyl group, for example a methoxycarbonyl, ethoxycarbonyl or tert-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine or 2-hydroxyethylamine, or with hydrazine.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

The protecting groups may be removed at any convenient stage in the synthesis using conventional techniques well known in the chemical art, or they may be removed during a later reaction step or work-up.

The skilled organic chemist will be able to use and adapt the information contained and referenced within the above references, and accompanying Examples therein and also the Examples herein, to obtain necessary starting materials, and products.

The removal of any protecting groups and the formation of a pharmaceutically-acceptable salt are within the skill of an ordinary organic chemist using standard techniques. Furthermore, details on the these steps has been provided hereinbefore.

When an optically active form of a compound of the invention is required, it may be obtained by carrying out one of the above procedures using an optically active starting material (formed, for example, by asymmetric induction of a suitable reaction step), or by resolution of a racemic form of the compound or intermediate using a standard procedure, or by chromatographic separation of diastereoisomers (when produced). Enzymatic techniques may also be useful for the preparation of optically active compounds and/or intermediates.

Similarly, when a pure regioisomer of a compound of the invention is required, it may be obtained by carrying out one of the above procedures using a pure regioisomer as a starting material, or by resolution of a mixture of the regioisomers or intermediates using a standard procedure.

According to a further aspect of the invention there is provided a pharmaceutical composition which comprises a compound of formula (I) as defined hereinbefore or a pharmaceutically-acceptable salt thereof, in association with a pharmaceutically-acceptable excipient or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

According to a further aspect of the present invention there is provided a compound of formula (I) or a pharmaceutically acceptable salt thereof as defined hereinbefore for use in a method of treatment of the human or animal body by therapy.

We have found that compounds of the present invention inhibit DPP-IV and are therefore of interest for their blood glucose-lowering effects.

A further feature of the present invention is a compound of formula (I) or a pharmaceutically-acceptable salt thereof for use as a medicament.

Conveniently this is a compound of formula (I), or a pharmaceutically-acceptable salt thereof, for use as a medicament for inhibiting DPP-IV in a warm-blooded animal such as a human being.

Particularly this is a compound of formula (I), or a pharmaceutically-acceptable salt thereof, for use as a medicament for treating diabetes mellitus in a warm-blooded animal such as a human being.

Thus according to a further aspect of the invention there is provided the use of a compound of formula (I), or a pharmaceutically-acceptable salt thereof in the manufacture of a medicament for use in the inhibition of DPP-IV in a warm-blooded animal such as a human being.

Thus according to a further aspect of the invention there is provided the use of a compound of formula (I), or a pharmaceutically-acceptable salt thereof in the manufacture of a medicament for use in the treatment of diabetes mellitus in a warm-blooded animal such as a human being.

According to a further aspect of the invention there is provided a pharmaceutical composition which comprises a compound of formula (I) as defined hereinbefore or a pharmaceutically-acceptable salt thereof, in association with a pharmaceutically-acceptable excipient or carrier for use in inhibiting DPP-IV in an warm-blooded animal, such as a human being.

According to a further aspect of the invention there is provided a pharmaceutical composition which comprises a compound of formula (I) as defined hereinbefore or a pharmaceutically-acceptable salt thereof, in association with a pharmaceutically-acceptable excipient or carrier for use in the treatment of diabetes mellitus in an warm-blooded animal, such as a human being.

According to a further feature of the invention there is provided a method for inhibiting DPP-IV in a warm-blooded animal, such as a human being, in need of such treatment which comprises administering to said animal an effective amount of a compound of formula (I) or a pharmaceutically-acceptable salt thereof as defined hereinbefore.

According to a further feature of the invention there is provided a method of treating diabetes mellitus in a warm-blooded animal, such as a human being, in need of such treatment which comprises administering to said animal an effective amount of a compound of formula (I) or a pharmaceutically-acceptable salt thereof as defined hereinbefore.

As stated above the size of the dose required for the therapeutic or prophylactic treatment of a particular disease state will necessarily be varied depending on the host treated, the route of administration and the severity of the illness being treated. Preferably a daily dose in the range of 1-50 mg/kg is employed. However the daily dose will necessarily be varied depending upon the host treated, the particular route of administration, and the severity of the illness being treated. Accordingly the optimum dosage may be determined by the practitioner who is treating any particular patient.

As stated above compounds defined in the present invention are of interest for their ability to inhibit the activity of DPP-IV. A compound of the invention may therefore be useful for the prevention, delay or treatment of a range of disease states including diabetes mellitus, more specifically type 2 diabetes mellitus (T2DM) and complications arising there from (for example retinopathy, neuropathy and nephropathy), impaired glucose tolerance (IGT), conditions of impaired fasting glucose, metabolic acidosis, ketosis, dysmetabolic syndrome, arthritis, osteoporosis, obesity and obesity related disorders, peripheral vascular disease, (including intermittent claudication), cardiac failure and certain cardiac myopathies, myocardial ischaemia, cerebral ischaemia and reperfusion, muscle weakness, hyperlipidaemias, Alzheimer's disease, atherosclerosis, infertility, polycystic ovary syndrome, various immunomodulatory diseases (such as psoriasis), HIV infection, inflammatory bowel syndrome, inflammatory bowel disease (such as Crohn's disease and ulcerative colitis.

In a further aspect, compounds of the formula (I) or their pharmaceutically acceptable salts may be administered in combination with other therapeutic agents in order to prevent, delay or treat the various disease states in which DPP-IV activity is implicated, including but not limited to those disease states listed above.

For example, in order to prevent, delay or treat type 2 diabetes mellitus, the compounds of the present invention or their pharmaceutically-acceptable salts may be administered in combination with a therapeutically effective amount of one or more other compounds of the formula (I) and/or one or more of the following agent(s):
1) Insulin and insulin analogues;
2) Insulin secretagogues including sulphonylureas, prandial glucose regulators and glucokinase activators;
3) Agents that improve incretin action (for example GLP-1 agonists);
4) Insulin sensitising agents including PPARgamma agonists and agents with combined PPARalpha and gamma activity;
5) Agents that modulate hepatic glucose balance (for example biguanides, fructose 1, 6 bisphosphatase inhibitors, glycogen phosphorylase inhibitors, glycogen synthase kinase inhibitors, glucokinase activators);
6) Agents designed to reduce the absorption of glucose from the intestine (for example alpha glucosidase inhibitors);
7) Agents that prevent the reabsorption of glucose by the kidney (sodium glucose transporter inhibitors);
8) Agents designed to treat the complications of prolonged hyperglycaemia (for example aldose reductase inhibitors, Protein Kinase C inhibitors);
9) Agents used to treat obesity (for example appetite suppressants) or that increase energy expenditure;
10) Anti-dyslipidaemia agents such as, HMG-CoA reductase inhibitors, PPAR alpha agonists (for example fibrates), PPAR delta agonists, bile acid sequestrants, cholesterol absorption inhibitors, bile acid absorption inhibitors, CETP inhibitors, inhibitors of lipolysis;
11) Antihypertensive agents such as, beta blockers, ACE inhibitors, Calcium antagonists, Angiotensin receptor antagonists, alpha receptor antagonists and diuretic agents;
12) Haemostasis modulators such as, antithrombotics, activators of fibrinolysis and antiplatelet agents, thrombin antagonists, factor Xa inhibitors, factor VIIa inhibitors, antiplatelet agents and anticoagulants;
13) Agents which antagonise the actions of glucagon; and
14) Anti-inflammatory agents, such as non-steroidal anti-inflammatory drugs and steroidal anti-inflammatory agents.

In addition to its use in therapeutic medicine, compounds of formula (I) and their pharmaceutically-acceptable salts are also useful as pharmacological tools in the development and standardisation of in-vitro and in-vivo test systems for the evaluation of the effects of inhibitors of DPP-IV in laboratory animals such as cats, dogs, rabbits, monkeys, rats and mice, as part of the search for new therapeutic agents.

As indicated above, all of the compounds, and their corresponding pharmaceutically-acceptable salts, are useful in inhibiting DPP-IV. The ability of the compounds of formula (I), and their corresponding pharmaceutically-acceptable acid addition salts, to inhibit DPP-IV may be demonstrated employing the caco-2 DPP-IV Assay which measures the ability of test compounds to inhibit DPP-IV activity from human colonic carcinoma cell extracts. The human colonic carcinoma cell line Caco-2 was obtained from the American Type Culture Collection (ATCC HTB 37). Differentiation of the cells to induce DPP-IV expression was accomplished as described by Reisher, et al. (Proc. Natl. Acad. Sci., Vol. 90, pgs. 5757-5761 (1993)). Cell extract is prepared from cells solubilized in 10 mM Tris HCI, 0.15 M NaCI, 0.04 t.i.u.aprotinin, 0.5% nonidet-P40, pH 8.0, which is centrifuged at 35,000 g for 30 min at 4° C. to remove cell debris.

The calorimetric assay is conducted by adding 20 μg solubilized Caco-2 protein or purified porcine kidney DPP-IV, in a final volume of 10 ul in assay buffer (25 mM Tris HCl pH 7.4, 140 mM NaCl, 10 mM KCl, 0.1% Triton-x-100) to microtiter plate wells. After a 10 min. incubation at room temperature, the reaction is initiated by adding 10 μl of 0.5 mM substrate (H-Glycine-Proline-pNA; pNA is p-nitroaniline). The final assay volume is 100 μl. The reaction is carried out at room temperature for 10 minutes after which time a 20 μI volume of sodium acetate buffer pH 4.5 is added to stop the reaction. Test compounds are typically added as 10 μl additions A standard curve of free p-nitro aniline is generated using 0-500 μM solutions of free pNA in assay buffer. The curve generated is linear and is used for interpolation of substrate consumption (catalytic activity in nmoles substrate cleaved/min). The endpoint is determined by measuring absorbance at 405 nm in a Labsystems microtiter plate reader.

Activity of CaCo2 extract is also measured employing a modified version of the assay described in Kubota, et al. (Clin. Exp. Immunol., Vol. 89, pgs. 192-197 (1992)). The assay is conducted by adding 10 μg solubilized Caco-2 protein, in a final volume of 10 ul assay buffer (25 mMHEPES, 140 mM NaCl, 80 mM MgCl₂, 0.1% Triton X-100, pH 7.4) to micro titer plate wells. After 10 min incubation at room temperature, the reaction is initiated by the addition of 10 μl of incubation buffer containing 0.5 mM substrate (H-Glycine-Proline-AMC; AMC is 7-amino-40-methylcoumarin). The plates are at room temperature (in the dark) for 10 min. Test compounds are typically added as 10 μl additions and the final assay buffer volume is 100 μl. The reaction is initiated by adding 10 μl of 0.5 mM substrate Gly-Pro-7-amino-4-trifluoromethylcoumarin for 10 minutes after which time a 20 μI volume of sodium acetate buffer pH 4.5 is added to stop the reaction. After the 10 min. reaction, florescence is measured using a Tecan Ultra fluorimeter (Excitation 360 nm Emission 465 nm). A standard curve of free AMC is generated using 0-50 μM solutions of free AMC in assay buffer. The curve generated is linear and is used for interpolation of substrate consumption (catalytic activity in nmoles substrate cleaved/min). The potency of the test compounds as DPP-IV inhibitors, expressed as IC₅₀, is calculated from 11-point, dose-response curves using a 4 parameter logistic function.

Using this assay the compounds generally show activity with IC₅₀<100 μM, preferably <10 μM and more preferably <1 μM. Example 1 showed an IC₅₀=0.58 μM.

The ability of the compounds of formula I, and their corresponding pharmaceutically acceptable acid addition salts, to inhibit DPP-IV may also be demonstrated by measuring the effects of test compounds on DPP-IV activity in human and rat plasma employing a modified version of the assay described above. Briefly, 5-10 μl of plasma are added to 96-well flat-bottom microtiter plates instead of CaCo2 extract, final assay volume is 100 μl. As with the previous assay, the potency of the test compounds as DPP-IV inhibitors, expressed as IC₅₀, is calculated from 11-point, dose-response curves using a 4 parameter logistic function.

In the above other pharmaceutical composition, process, method, use and medicament manufacture features, the alternative and preferred embodiments of the compounds of the invention described herein also apply.

### EXAMPLES

The invention will now be illustrated by the following Examples in which, unless stated otherwise:
(i) temperatures are given in degrees Celsius (° C.); operations were carried out at room or ambient temperature, that is, at a temperature in the range of 18-25° C. and under an atmosphere of an inert gas such as argon;
(ii) organic solutions were dried over anhydrous magnesium sulphate; evaporation of solvent was carried out using a rotary evaporator under reduced pressure (600-4000 Pascals; 4.5-30 mmHg) with a bath temperature of up to 60° C.;
(iii Purification by chromatography generally refers to flash column chromatography, on silica unless otherwise stated. Column chromatography was generally carried out using prepacked silica cartridges (from 4 g up to 400 g) such as Redisep™ (available, for example, from Presearch Ltd, Hitchin, Herts, UK) or Biotage (Biotage UK Ltd, Hertford, Herts, UK), eluted using a pump and fraction collector system. Alternatively chromatography was carried out using ISOLUTE prepacked silica cartridges (10 g to 50 g) (available for, for example, from IST, Dyffryn Business Park), in this case elution and fraction collection was carried out manually.
(iv) in general, the course of reactions was followed by TLC and reaction times are given for illustration only;
(v) yields are given for illustration only and are not necessarily those which can be obtained by diligent process development; preparations were repeated if more material was required;
(vi) the structures of the end-products of the Formula (I) were confirmed by nuclear (generally proton) magnetic resonance (NMR) with a field strength (for proton) of 300 MHz (generally using a Varian Gemini 2000) or 400 MHz (generally using a Bruker Avance DPX400), unless otherwise stated, and mass spectral techniques; proton magnetic resonance chemical shift values were measured on the delta scale and peak multiplicities are shown as follows: s, singlet; d, doublet; t, triplet; m, multiplet; br, broad; q, quartet, quin, quintet; using perdeuterio dimethyl sulphoxide (DMSO-d₆) as solvent, unless otherwise stated
(vii) chemical symbols have their usual meanings; SI units and symbols are used;
(viii) solvent ratios are given in volume : volume (v/v) terms;
(ix) Mass spectra (MS) data was generated on an LCMS system where the HPLC component comprised generally either a Agilent 1100 or Waters Alliance HT (2790 & 2795) equipment and was run on a Phemonenex Gemini C18 5 μm, 50×2 mm column (or similar) eluting with either acidic eluent (for example, using a gradient between 0-95% water/acetonitrile with 5% of a 1% formic acid in 50:50 water: acetonitrile (v/v) mixture; or using an equivalent solvent system with methanol instead of acetonitrile), or basic eluent (for example, using a gradient between 0-95% water/acetonitrile with 5% of a 0.1% 880 Ammonia in acetonitrile mixture); and the MS component comprised generally a Waters ZQ spectrometer. Chromatograms for Electrospray (ESI) positive and negative Base Peak Intensity, and UV Total Absorption Chromatogram from 220-300 nm, are generated and values for m/z are given; generally, only ions which indicate the parent mass are reported and unless otherwise stated the value quoted is (M+H)⁻;
(x) Suitable microwave reactors include “Smith Creator”, “CEM Explorer”, “Biotage Initiator sixty” and “Biotage Initiator eight”.
(xi) The following abbreviations may be used:
   Et₂O diethyl ether
   DMF dimethylformamide
   DCM dichloromethane
   DME dimethoxyethane
   MeOH methanol
   EtOH ethanol
   H₂O water
   TFA trifluoroacetic acid
   THF tetrahydrofuran
   DMSO dimethylsulfoxide
   HOBt 1-hydroxybenzotriazole
   EDCI (EDAC) 1-ethyl-3-(3-dimethylaminopropyl)carbodi-imide hydrochloride
   DIPEA diisopropylethylamine
   DEAD diethyl azodicarboxylate
   EtOAc ethyl acetate
   NaHCO₃ sodium bicarbonate
   HATU O-(benzotriazol-1-yl)-N,N,N,N-tetramethyluronium hexafluorophosphate
   DMTMM 4-(4,6-dimethoxy-1,3,5-triazin-2-yl)-4-methylmorpholinium chloride
   HPLC high performance liquid chromatography
   MPLC medium pressure liquid chromatography
   Boc tert-butyloxycarbonyl
   NH₃ ammonia
   NaOH sodium hydroxide
   DMA N,N-dimethylacetamide
   HBTU O-benzotriazolyl-N,N,N′,N′-tetramethyluronium

### Example 1

### (3R)-3-Amino-4-(2-fluorophenyl)-N-(2-oxo-1,2,3,4-tetrahydroquinolin-3-yl)butanamide

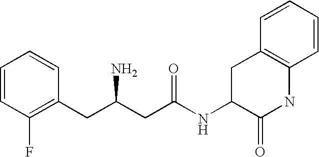

tert-Butyl{(1R)-1-(2-fluorobenzyl)-3-oxo-3-[(2-oxo-1,2,3,4-tetrahydroquinolin-3-yl)amino]propyl}carbamate (Intermediate 1, 1.27 mmol, 650 mg) was dissolved in 1,4-dioxane (30 ml) and treated with 4M solution of HCl in 1,4-dioxane (10 ml). The resulting solution was stirred at ambient temperature for 4 days as a precipitate formed. Volatiles were removed under reduced pressure. The residue obtained was partitioned between EtOAc (250 ml) and excess saturated aqueous sodium bicarbonate. The organics were washed with saturated brine and dried (MgSO₄), then filtered and evaporated to yield the title compound (316 mg, 93%) as a colourless solid.

¹H NMR: 2.20 (m, 4), 2.70 (m, 2H), 2.90 (m, 1H), 3.00 (m, 1H), 3.33 (m, 1H), 4.23 (m, 1H), 6.88 (m, 2H), 7.15 (m, 4H), 7.26 (m, 2H), 8.29 (d, 1H), 10.26 (s, 1H); MS (M+H)⁺342.

### Intermediate 1: tert-Butyl{(1R)-1-(2-fluorobenzyl)-3-oxo-3-[(2-oxo-1,2,3,4-tetrahydroquinolin-3-yl)amino]propyl}carbamate

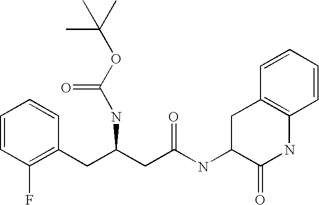

The hydrochloride salt of 3-amino-3,4-dihydro-2(1H)-quinolinone (CAS Reg. No: 35849-31-1, 1.69 mmol, 335 mg) was suspended in anhydrous DCM (10 ml) and treated with DIPEA (348 μl, 2 mmol) to give a clear solution. This further treated with (tert-Butyl[(1R)-3-[(2,5-dioxopyrrolidin-1-yl)oxy]-1-(2-fluorobenzyl)-3-oxopropyl]carbamate (Intermediate 2, 1.69 mmol, 664 mg). The reaction mixture was stirred at ambient temperature for approximately 66 h. The resulting suspension was treated with 250 ml water plus 250 ml DCM. After vigorous stirring, the insoluble material was collected, washed with DCM and dried in vacuo, to give the title compound (580 mg, 78%) as a colourless solid.

¹H NMR: 1.29 (s, 9H), 2.38 (m, 2), 2.70 (m, 1H), 2.88 (m, 2H), 3.03 (m, 1H), 4.08 (m, 1H). 4.46 (m, 1H), 6.68 (m, 1H), 6.88 (d, 1H), 6.95 (m, 1H), 7.12 (m, 2H), 7.19 (m, 2H), 7.27 (m, 2H), 8.17 (dd, 1H), 10.30 (s, 1H); MS (M+Na)⁺464, (M−H)⁻440.

### Intermediate 2: tert-Butyl[(1R)-3-[(2,5-dioxopyrrolidin-1-yl)oxy]-1-(2-fluorobenzyl)-3-oxopropyl]carbamate

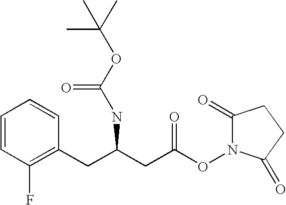

A mixture of (3R)-3-[(tert-butoxycarbonyl)amino]-4-(2-fluorophenyl)butanoic acid (Peptech, CAS Registry No 218608-98-1, 10.0 g, 33.8 mmol) and N-hydroxysuccinimide (4.09 g, 35.5 mmol) in DCM (125 ml) was treated with EDAC (7.78 g, 40.6 mmol). The mixture was stirred overnight at room temperature. The mixture was diluted with DCM and washed successively with 1M HCl solution and aqueous sodium bicarbonate. The organic solution was dried (MgSO₄) and concentrated under reduced pressure. The resulting solid was purified by MPLC on silica (Isco Companion®; gradient elution from 100% DCM to 20% ethyl acetate/DCM) to give the title compound as a colourless solid (7.28 g, 55%) ¹H NMR (CDCl₃): 1.380 (s, 9H), 2.85 (s, 6H), 2.92-3.12 (m, 2H), 4.22-4.38 (m, 1H), 4.90-5.08 (m, 1 H), 6.99-7.13 (m, 2H), 7.18-7.30 (m, 2H); MS (+ve ESP): 417 (M+Na⁺).

### Example 2

### (3R)-3-Amino-4-(2-fluorophenyl)-N-(2-oxo-1,2,3,4-tetrahydro-1,5-naphthyridin-3-yl)butanamide

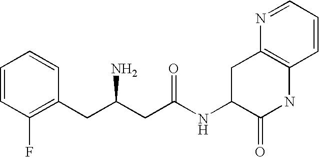

tert-Butyl{(1R)-1-(2-fluorobenzyl)-3-oxo-3-[(2-oxo-1,2,3,4-tetrahydro-1,5-naphthyridin-3-yl)amino]propyl}carbamate (Intermediate 3, 1.6 mmol, 704 mg) was dissolved in 1,4-dioxane (16 ml) and treated with a 4M solution of HCl in 1,4-dioxane (4 ml), to yield an immediate precipitate. The resulting solution was stirred at ambient temperature for 4 days. Volatiles were removed under reduced pressure. The residue obtained was partitioned between EtOAc (250 ml) and excess saturated aqueous NaHCO₃. The organics were washed with saturated brine and dried (MgSO₄). Filtered and evaporated to yield the title compound (184 mg, 54%) as a colourless solid.

¹H NMR 1.75, (broad, 2H), 2.18 (m, 1), 2.25 (m, 1H), 2.70 (m, 2H), 3.13 (m, 2), 4.63 (m, 1H), 7.15 (m, 4H), 7.29 (m, 2H), 8.11 (m, 1H), 8.43 (d, 1H), 10.37 (s, 1H); MS: (M+H)⁺343.

### Intermediate 3: tert-Butyl{(1R)-1-(2-fluorobenzyl)-3-oxo-3-[(2-oxo-1,2,3,4-tetrahydro-1,5-naphthyridin-3-yl)amino]propyl}carbamate

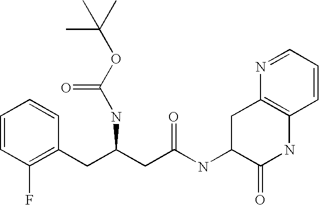

The dihydrochloride salt of 3-amino-3,4-dihydro-1,5-naphthyridin-2(1H)-one (CAS Reg. No: 600157-67-3, [PCT Int. Appl. (2003), WO 2003074532], 2.00 mmol, 673 mg) was suspended in anhydrous DCM (20 ml) and treated with DIPEA (697 μl, 4 mmol) to give a clear solution. This further treated with tert-butyl[(1R)-3-[(2,5-dioxopyrrolidin-1-yl)oxy]-1-(2-fluorobenzyl)-3-oxopropyl]carbamate (Intermediate 2, 789 mg, 2.00 mmol) with additional DCM (10 ml). The almost clear solution was stirred at ambient temperature for 1.5 h. The resulting clear solution was treated with a mixture of DCM (120 ml) and water (150 ml) to yield a gelatinous solid. This was collected, washed with DCM and dried on the filter to yield the title compound as an amorphous solid (767 mg, 86%).

¹H NMR: 1.29 (s, 9H), 2.38 (m, 2), 2.68 (m, 1H), 2.85 (m, 1H), 3.08 (m, 2H), 4.03 (m, 1H). 4.60 (m, 1H), 6.67 (m, 1H), 7.10 (m, 2H), 7.18 (d, 2H), 7.25 (m, 2H), 8.09 (m, 1H), 8.10 (m, 1H), 10.36 (s, 1H); MS: (M+Na)⁺465, (M−H)⁻441.

Single isomers of 3-amino-3,4-dihydro-1,5-naphthyridin-2(1H)-one may be made by deprotection of each single isomer of the N-Boc protected compounds. These protected compounds may be made in the following manner:

### tert-Butyl[(3S)-2-oxo-1,2,3,4-tetrahydro-1,5-naphthyridin-3-yl]carbamate

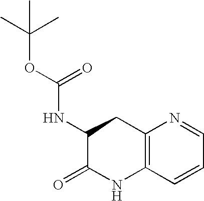

To a cooled oven dried flask under nitrogen was added zinc dust (2.45 g, 7.44 mmol) followed by anhydrous DMF (9.1 ml) and chlorotrimethylsilane (1.25 ml, 9.89 mmol). The suspension was stirred vigorously for 10 mins, allowed to stand for 40 mins, the solvent was removed via a syringe and the residue heated under vacuum with a heat gun for 5 mins and allowed to cool. A solution of methyl N-(tert-butoxycarbonyl)-3-iodo-L-alaninate (2.45 g, 7.44 mmol) in anhydrous DMF (9.1 ml) was added and the suspension was stirred vigorously for 30 mins. Dichlorobis(triphenylphosphine)palladium (282 mgs, 0.401 mmol) and 3-amino-2-bromopyridine (1.71 g, 9.90 mmol) were added and the reaction mixture was allowed to stir at ambient temperature for 48 hrs. The mixture was filtered through a pad of celite, washed with EtOAc (2×200 ml), the filtrate was washed with brine (2×100 ml), the organic extract was dried (MgSO₄) and concentrated to leave crude product. The residue was purified on a 120 g Redisep silica cartridge (Isco Companion®; eluting with 30-80% EtOAc-isohexane) to provide the title compound as a solid (1.04 g, 54%); ¹H NMR (CDCl₃) 1.49 (s, 9H), 3.09 (t, 1H), 3.62-3.70 (dd, 1H), 4.47-4.57 (m, 1H), 5.41 (d, 1H), 7.10-7.15 (m, 2H), 8.25-8.27 (m, 1H), 8.38 (brs, 1H); MS 286 (M+Na)⁺, 264 (MH)⁺.

### tert-Butyl[(3R)-2-oxo-1,2,3,4-tetrahydro-1,5-naphthyridin-3-yl]carbamate

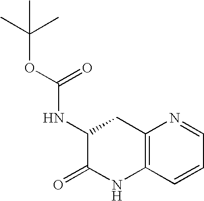

tert-Butyl[(3R)-2-oxo-1,2,3,4-tetrahydro-1,5-naphthyridin-3-yl]carbamate was prepared in 52% yield using the procedure described above except methyl N-(tert-butoxycarbonyl)-3-iodo-D-alaninate was used instead of methyl N-(tert-butoxycarbonyl)-3-iodo-L-alaninate; ¹H NMR (CDCl₃) 1.49 (s, 9H), 3.09 (t, 1H), 3.62-3.69 (dd, 1H), 4.47-4.57 (m, 1H), 5.43 (d, 1H), 7.14 (m, 2H), 8.26 (d, 1H), 8.48 (brs, 1H); MS 286 (M+Na)⁺, 264 (MH)⁺.

### Example 3

### (R)-3-Amino-4-(2-fluorophenyl)-N-(2-oxo-1,2,3,4-tetrahydro-1,8-naphthyridin-3-yl)-butanamide dihydrochloride

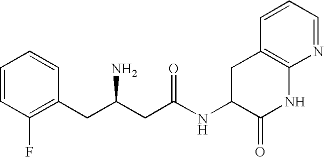

{(R)-2-(2-Fluorophenyl)-1-[(2-oxo-1,2,3,4-tetrahydro-1,8-naphthyridin-3-ylcarbamoyl)methyl]ethyl}carbamic acid tert-butyl ester (Intermediate 4, 100 mg, 0.226 mmol) was dissolved in a 4M solution of HCl in 1,4-dioxane (5 ml, 20 mmol). The reaction was left to stir overnight then evaporated in vacuo. The resulting solid was triturated with ether then filtered and dried under high vac to yield the product as the dihydrochloride salt (53 mg, 57%).

¹H NMR (DMSO-d₆, acetic acid-d₄ added): 2.60 (m, 2H), 2.95 (m, 2H), 3.10 (m, 2H), 3.75 (m, 1H), 4.50 (m, 1H), 6.95 (m, 1H), 7.15 (m, 2H), 7.35 (m, 2H), 7.60 (t, 1H), 8.15 (m, 1H)8.4 (s, 1H); MS: 342 (M+H)⁺.

### Examples 3a and 3b

### Diastereoisomers of (R)-3-amino-4-(2-fluorophenyl)-N-(2-oxo-1,2,3,4-tetrahydro-1,8-naphthyridin-3-yl)-butanamide dihydrochloride (AZ12305149 & AZ12310286)

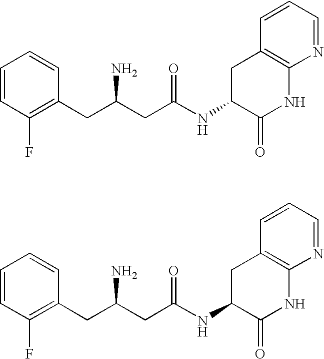

A sample of (R)-3-Amino-4-(2-fluorophenyl)-N-(2-oxo-1,2,3,4-tetrahydro-1,8-naphthyridin-3-yl)-butanamide (Example 3) was separated into its component diastereoisomers using prep HPLC (Phenomenex column, Luna 10u C18(2) 100A, 150×21.2 mm, eluting with 5 to 23% CH₃CN over 20 minutes). First isomer RT 11.68 minutes, Second isomer RT 12.17 minutes. The absolute stereochemistry of the isomers was not determined.

Spectral data for first eluted: ¹H NMR (CD₃OD): 2.56 (dd, 1H), 2.75 (dd, 1H), 3.08 (m, 2H), 3.16 (m, 2H), 3.89 (m, 1H), 4.73 (dd, 1H), 7.05 (m, 1H), 7.19 (m, 2H), 7.38 (m, 2H), 7.63 (d, 1H), 8.17 (d, 1H); MS (+ve ESP): 343 (M+H)⁺.

Spectral data for second elute: ¹H NMR (CD₃OD): 2.60 (dd, 1H), 2.67 (dd, 1H), 2.81 (d, 1H), 2.90 (d, 1H), 3.04 (m, 2H), 3.18 (m, 2H), 3.88 (m, 1H), 4.80 (m, 1H), 7.00 (m, 1H), 7.17 (m, 2H), 7.38 (m, 2H), 7.63 (d, 1H), 8.17 (m, 1H); MS (+ve ESP): 343 (M+H)⁺.

### Intermediate 4: tert-Butyl{(1R)-1-(2-fluorobenzyl)-3-oxo-3-[(2-oxo-1,2,3,4-tetrahydro-1,8-naphthyridin-3-yl)amino]propyl}carbamate

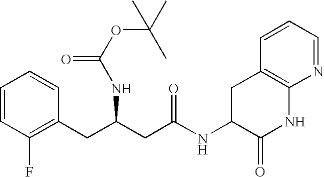

In a microwave tube was placed 3-amino-3,4-dihydro-1,8-naphthyridin-2(1H)-one (Intermediate 5, 80 mg, 0.4 mmol), tert-butyl[(1R)-3-[(2,5-dioxopyrrolidin-1-yl)oxy]-1-(2-fluorobenzyl)-3-oxopropyl]carbamate (Intermediate 2, 158 mg, 0.4 mmol) and triethylamine (73 μl, 0.52 mmol) in dioxan (5 ml). The reaction was heated by microwave at 150° C. for 25 minutes. The reaction was repeated on the same scale under the same conditions and the two crude reaction mixtures were then combined for work up and purification. The reaction mixture was evaporated under reduced pressure and the resulting residue was partitioned between EtOAc (70 ml) and 1M hydrochloric acid (30 ml). The organic layer was separated and washed with 1M hydrochloric acid (30 ml), saturated sodium bicarbonate solution (30 ml), water (2×30 ml) and brine (30 ml) then dried (MgSO₄), filtered and the solvent evaporated to leave a solid. This solid was purified by column chromatography (eluant DCM to 2% MeOH/DCM) to yield the product (105 mg, 59%). ¹H NMR :1.30 (br s, 9H), 2.35 (m, 2H), 2.70 (m, 1H), 2.90 (d, 2H), 3.05 (m, 1H), 4.10 (m, 1H), 4.50 (m, 1H), 6.70 (m, 1H), 7.00 (m, 1H), 7.10 (m, 2H), 7.30 (m, 2H), 7.60 (m, 2H), 8.10 (m, 1H), 8.20 (m, 1H), 10.7 (m, 1H); MS: 465 (M+Na)^{+.}

### Intermediate 5: 3-amino-3,4-dihydro-1,8-naphthyridin-2(1H)-one

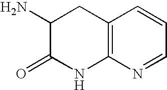

Prepared from Intermediate 6 following the method described below for the conversion of Intermediate 14 into Intermediate 13. ¹H NMR (CDCl₃): 2.90 (t, 1H), 3.15 (dd, 1H), 4.70 (m, 1H), 7.00 (m, 1H), 7.50 (d, 1H), 8.30 (d, 1H), 9.85 (br s, 1H); MS: 164 (M+H)⁺.

### Intermediate 6: tert-Butyl(2-oxo-1,2,3,4-tetrahydro-1,8-naphthyridin-3-yl)carbamate

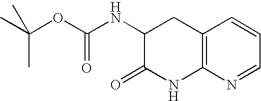

Methyl (2Z)-3-(2-aminopyridin-3-yl)-2-[(tert-butoxycarbonyl)amino]acrylate (Intermediate 7, 3.17 g, 10.8 mmol) was suspended in ethanol (200 ml) and palladium on carbon catalyst (500 mg, 10% w/w) was added. The mixture was stirred under 1 atmosphere of hydrogen at ambient temperature for 24 hours. After removing the catalyst by filtration through Celite the filtrate was concentrated under reduced pressure to give a white solid which was purified by chromatography on silica gel eluting with isohexane containing an increasing proportion of ethyl acetate (0-100%). The volatiles were evaporated under reduced pressure, then the resulting solid was triturated with ether and the product collected by filtration to give the title compound (1.3 g, 46%) as a solid.

¹H NMR: 1.4 (s,9H); 3.0 (m,2H); 4.2 (m, 1H); 6.9 (m,2H), 7.6 (d, 1H); 8.1 (d,1H), 10.7 (s, 1H); MS: 264.

### Intermediate 7: Methyl (2Z)-3-(2-aminopyridin-3-yl)-2-[(tert-butoxycarbonyl)amino]acrylate

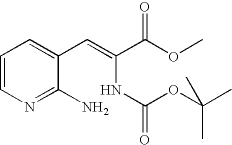

Methyl[(tert-butoxycarbonyl)amino](dimethoxyphosphoryl)acetate (5.47 g, 18.0 mmol) was dissolved in dry THF (135 ml) and cooled to −78° C. under nitrogen. Tetramethylguanidine (2.42 ml, 19.0 mmol) was added and the solution stirred at −78° C. for a further 15 mins. A solution 2-aminonicotinaldehyde (2.25 g, 18 mmol) in dry THF (50 ml) was then added dropwise. After the addition was complete stirring was continued at ambient temperature for a further 18 hours. The solution was then diluted with water (100 ml) and extracted with ethyl acetate (3×100 ml). The combined extracts were washed with water (2×100 ml) and brine (100 ml), dried (MgSO₄) and evaporated under reduced pressure to give a yellow solid. This was further purified by chromatography on silica gel eluting with isohexane containing an increasing proportion of ethyl acetate (50-100%) to give the title compound (3.19 g, 59%) as a yellow solid.

¹H NMR: 1.4 (s, 9H); 3.7 (m, 3H); 6.05 (s, 2H); 6.6 (m, 1H); 7.0 (bs, 1H); 7.6 (d, 1H); 7.9 (d, 1H), 8.5 (bs, 1H); MS: 394.

### Example 4

### (R)-3-Amino-4-(4-fluorophenyl)-N-(2-oxo-1,2,3,4-tetrahydro-1,8-naphthyridin-3-yl)butanamide

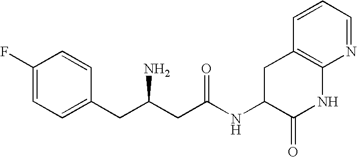

tert-Butyl{(1R)-1-(4-fluorobenzyl)-3-oxo-3-[(2-oxo-1,2,3,4-tetrahydro-1,8-naphthyridin-3-yl)amino]propyl}carbamate (Intermediate 8) was deprotected using the same procedure as described in Example 3. ¹H NMR (DMSO-d₆, acetic acid-d₄added): 2.85 (m, 2H), 3.00 (m, 2H), 3.60 (m, 1H), 4.60 (m, 1H), 6.95 (m, 1H), 7.15 (t, 2H), 7.30 (m, 2H), 7.60 (t, 1H), 8.10 (m, 1H); MS: 342 (M+H)⁺.

### Intermediate 8: tert-Butyl{(1R)-1-(4-fluorobenzyl)-3-oxo-3-[(2-oxo-1,2,3,4-tetrahydro-1,8-naphthyridin-3-yl)amino]propyl}carbamate

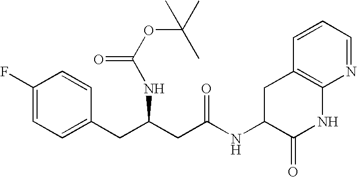

In a microwave tube was placed 3-amino-3,4-dihydro-1,8-naphthyridin-2(1H)-one (Intermediate 6, 56 mg, 0.24 mmol), (3R)-3-[(tert-butoxycarbonyl)amino]-4-(4-fluorophenyl)butanoic acid (Peptech, CAS Registry No. 218609-00-8 , 70 mg, 0.24 mmol), triethylamine (72 μl, 0.52 mmol), HOBT (35 mg, 0.26 mmol) and EDAC (45 mg, 0.235 mmol) in acetonitrile (5 ml). The reaction was heated by microwave to 100° C. for 10 minutes. The reaction was repeated on the same scale under the same conditions and the two crude reaction mixtures were then combined for work up and purification. The reaction mixture was evaporated in vacuo to yield a pale yellow solid. This solid was partitioned between water (˜30 ml) and DCM (˜50 ml). The layers were separated and the aqueous was re-extracted with DCM (2×˜50 ml). The organic layers were combined, washed with water (˜30 ml) and brine (˜30 ml) then evaporated to a solid. This solid was triturated with ether then filtered to yield the product as an off white solid (129 mg, 62%). 1H NMR: 1.35 (s, 9H), 2.40 (m, 2H), 2.70 (m, 1H), 2.90 (br m, 2H), 3.10 (m, 1H), 3.20 (d, 2H), 3.35 (s, 2H), 4.00 (br m, 1H), 4.10 (m, 1H), 4.60 (m, 1H), 6.75 (t, 1H), 7.05 (m, 1H), 7.10 (t, 2H), 7.25 (m, 2H), 7.65 (br d, 1H), 8.20 (d, 1H), 8.25 (m, 1H); MS: 465 (M+Na)⁺.

### Example 5

### (R)-3-Amino-4-(2-fluorophenyl)-N-(2-oxo-1,2,3,4-tetrahydro-1,7-naphthyridin-3-yl)-butanamide

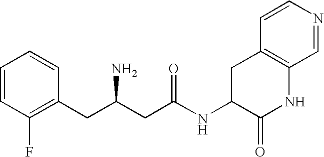

In a microwave tube was placed 3-amino-3,4-dihydro-1H-1,7-naphthyridin-2-one dihydrochloride (Intermediate 9, 80 mg, 0.34 mmol), tert-Butyl[(1R)-3-[(2,5-dioxopyrrolidin-1-yl)oxy]-1-(2-fluorobenzyl)-3-oxopropyl]carbamate (Intermediate 2, 134 mg, 0.34 mmol) and triethylamine (99 μl, 0.71 mmol) in acetonitrile (5 ml). The reaction was heated by microwave at 150° C. for 15 minutes. The reaction was repeated on the same scale under the same conditions and the two crude reaction mixtures were then combined for work up and purification. The reaction mixture was evaporated under reduced pressure to yield a pale brown solid. This solid was triturated with water then filtered and dried under high vac to yield the crude Boc-protected compound. This compound was suspended in dioxane (5 ml) and treated with a 4M solution of HCl in 1,4-dioxane (15 ml). The resulting reaction was stirred over night at ambient temperature. The volatiles were removed under reduced pressure and the resulting residue was partitioned between 1M NaOH solution (˜2 ml) and DCM (˜30 ml). The layers were separated and the aqueous was re-extracted with DCM (2×˜40 ml). The combined organic layers were evaporated to yield a residue. This residue was loaded onto a 10 g SCX-2 column and eluted with MeOH (˜100 ml). The product was then eluted off using 1% ammonia/MeOH. The fractions containing product were combined and evaporated to yield the product as the free base (125 mg, 54%). ¹H NMR: 2.15 (m, 1H), 2.20 (m, 1H), 2.65 (m, 2H), 2.95 (m, 1H), 3.05 (m, 1H), 4.50 (m, 1H), 7.15 (m, 2H), 7.25 (br m, 3H), 8.15 (m, 2H), 8.40 (d, 1H), 10.45 (br s, 1H); MS: 343 (M+H)⁺.

### Intermediate 9: 3-Amino-3,4-dihydro-1,7-naphthyridin-2(1H)-one dihydrochloride

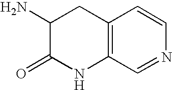

Prepared from Intermediate 10 following the method described below for the conversion of Intermediate 14 into Intermediate 13. ¹H NMR: 3.35 (m, 1H), 3.50 (m, 1H), 4.35 (m, 1H), 7.80 (d, 1H), 8.35 (s, 1H), 8.40 (d, 1H), 8.85 (br s, 3H), 11.40 (br s, 1H); MS: 164 (M+H)⁺.

### Intermediate 10: tert-Butyl(2-oxo-1,2,3,4-tetrahydro-1,7-naphthyridine-3-yl)carbamate

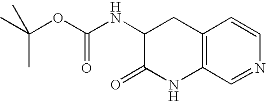

Methyl 2-[(tert-butoxycarbonyl)amino]-3-(3-nitropyridin-4-yl)acrylate (Intermediate 11, 10:1 mixture of Z/E isomers) (1.57 g, 4.83 mmol) was dissolved in ethanol and 10% palladium on carbon catalyst (250 mg) was added. The mixture was stirred under 1 atmosphere of hydrogen at ambient temperature for 6 hours. After removing the catalyst by filtration through Celite, the filtrate was concentrated under reduced pressure to give a yellow oil which was purified by column chromatography (Eluent DCM/MeOH gradient 0-10%) to give tert-butyl(2-oxo-1,2,3,4-tetrahydro-1,7-naphthyridine-3-yl)carbamate (284 mg, 22%).

¹H NMR: 1.4 (s, 9H); 3.0 (m, 2H); 4.2 (m, 1H); 7.0 (d, 1H); 7.2 (d, 1H); 8.1 (m, 2H); 10.36 (s, 1H); MS: 264.

### Intermediate 11: Methyl 2-[(tert-butoxycarbonyl)amino]-3-(3nitropyridin-4-yl)acrylate

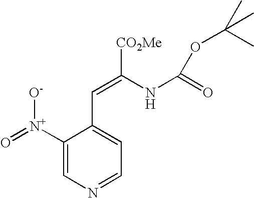

Methyl[(tert-butoxycarbonyl)amino](dimethoxyphosphoryl)acetate (1.73 g, 5.82 mmol) was dissolved in dry THF (20 ml) and cooled to −78 ° C. under nitrogen. Tetramethylguanidine (638 mg, 5.55 mmol) was added and the solution stirred at −78 ° C. for a further 10 mins. A solution of 3-nitroisonicotinaldehyde (Intermediate 12, 804 mg, 5.29 mmol) in dry THF (5 ml) was added dropwise. The resulting deep red solution was stirred for 2 hrs. at −78° C., then poured into a mixture of ethyl acetate (100 ml) and water (50 ml). The organic layer was separated, washed with water (2×50 ml) and brine (25 ml), dried (MgSO₄) and evaporated under reduced pressure to give a yellow oil, which was purified by column chromatography (EtOAc: isohexane 1:1) to give methyl-2-[(tert-butoxycarbonyl)amino]-3-(3-nitropyridin-4-yl)acrylate as a 10:1 mixture of Z/E isomers (1.57 g, 92%).

¹H NMR: 1.3 (s, 9H); 1.4 (s, 0.9H); 3.55 (s, 0.3H); 3.8 (s, 3H); 6.6 (s, 0.1H); 7.2 (s, 1H); 7.25(d, 0.1H); 7.5 (d, 1H); 8.75 (d, 0.1H); 8.8 (s, 1.1H); 8.85 (d, 1H); 9.2 (s, 0.1H); 9.25 (s, 1H); MS: 322.

### Intermediate 12: 3-Nitroisonicotinaldehyde

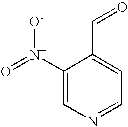

4-Methyl-3-nitropyridine (1.43 g, 10.36 mmol) was dissolved in dry DMF (5 ml) and dimethylformamide dimethyl acetal (2.0 g, 16.8 mmol) was added. The mixture was heated under nitrogen at 140° C. for 2 hours and then evaporated under reduced pressure to give (E)-N,N-dimethyl-2-(3-nitropyridin-4-yl)ethyleneamine as a dark red solid. This was added in one portion at ambient temperature to a stirred solution of sodium periodate (6.61 g, 31 mmol) in THF/Water 1:1 (100 ml). After stirring for 2 hr at ambient temperature the reaction mixture was filtered and the solid washed with ethyl acetate (100 ml). The washings were combined with the filtrate and organic layer separated. The aqueous was extracted with ethyl acetate (2×100 ml) and the combined organic layers were washed with saturated aqueous sodium bicarbonate (100 ml) and brine (100 ml), dried (MgSO₄) and evaporated under reduced pressure to give a brown solid which was purified by column chromatography (DCM) to give 3-nitroisonicotinaldehyde (960 mg, 61%).

¹H NMR: 7.8 (d, 1H); 9.15 (d, 1H); 9.4(s, 1H); 10.4 (s, 1H)

### Example 6

### (R)-3-Amino-4-(4-fluorophenyl)-N-(2-oxo-1,2,3,4-tetrahydro-1,7-naphthyridin-3-yl)butanamide

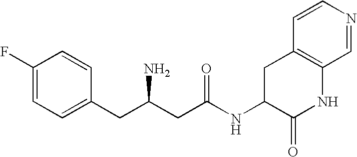

In a microwave tube was placed 3-amino-3,4-dihydro-1H-1,7-naphthyridin-2-one dihydrochloride (Intermediate 9, 63 mg, 0.27 mmol), (R)-3-tert-butoxycarbonylamino-4-(4-fluorophenyl)butanoic acid (80 mg, 0.27 mmol), triethylamine (79 μl, 0.56 mmol), HOBT (40 mg, 0.30 mmol) and EDAC (52 mg, 0.27 mmol) in acetonitrile (5 ml). The reaction was heated by microwave to 100° C. for 12 minutes. The reaction was repeated on the same scale under the same conditions and the two crude reaction mixtures were then combined for work up and purification. The reaction mixture was evaporated in vacuo to yield a pale yellow solid. This solid was triturated with water then filtered and dried under high vacuum to yield the crude Boc-protected compound. This compound was suspended in dioxan (5 ml) and treated with 4M HCl in dioxan (15 ml). The resulting reaction was stirred over night at ambient temperature. The volatiles were removed under reduced pressure and the resulting residue was partitioned between 1M NaOH solution (˜2 ml) and DCM (˜30 ml). The layers were separated and the aqueous was re-extracted with DCM (2×˜40 ml). The combined organic layers were evaporated to yield a residue. This residue was loaded onto a 10 g SCX-2 column and eluted with MeOH (˜100 ml). The product was then eluted off using 1% ammonia in methanol. The fractions containing product were combined and evaporated to yield the product as the free base (121 mg, 66%). ¹H NMR: 2.10 (m, 1H), 2.20 (m, 1H), 2.55 (m, 1H), 2.65 (m, 1H), 3.00 (t, 1H), 3.10 (m, 1H), 3.10-3.30 (br m, 3H), 4.50 (m, 1H), 7.10 (t, 2H), 7.25 (m, 3H), 8.10 (m, 2H), 8.40 (m, 1H); MS: 343 (M+H)⁺.

### Example 7

### (R)-3-Amino-4-(2-fluorophenyl)-N-(2-oxo-1,2,3,4-tetrahydro-1,6-naphthyridin-3-yl)butanamide

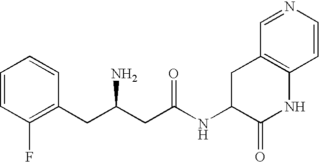

In a microwave tube was placed 3-amino-3,4-dihydro-1,6-naphthyridin-2(1H)-one dihydrochloride (Intermediate 13, 80 mg, 0.34 mmol), tert-Butyl[(1R)-3-[(2,5-dioxopyrrolidin-1-yl)oxy]-1-(2-fluorobenzyl)-3-oxopropyl]carbamate (134 mg, 0.34 mmol) and triethylamine (99 μl, 0.71 mmol) in acetonitrile (5 ml). The reaction was heated by microwave at 150° C. for 15 minutes. The reaction was repeated on the same scale under the same conditions and the two crude reaction mixtures were then combined for work up and purification. The reaction mixture was evaporated under reduced pressure to yield a pale brown solid which was partitioned between water and a large volume of a mixture of EtOAc and DCM, a little solid remaining undissolved. The organic layer was separated then washed and with citric acid and water. The citric acid and water extracts were combined and evaporated in vacuo to yield an orange gum, which was dissolved in THF and treated with 4M HCl in dioxan. The reaction was stirred at room temperature over night then evaporated to a residue which was partitioned between DCM (˜20 ml) and 2M NaOH (˜2 ml). The layers were separated and the aqueous was re-extracted with DCM (˜10 ml), the combined organic layers were then evaporated to a residue which was purified using SCX-2 chromatography (10 g SCX-2, MeOH then 1% ammonia/MeOH). The resulting material was dissolved in EtOH and treated with 2M HCl in ether (˜1 ml). The solvent was evaporated and the resulting solid was triturated with ether then filtered and dried under high vac to yield the product as the hydrochloride salt (18 mg, 7%). ¹H NMR (DMSO-d6, -acetic acid-d₄ added): 2.95 (m, 1H), 3.05 (m, 2H), 3.20 (m 1H), 3.70 (m, 1H), 4.60 (m, 1H), 7.20 (m, 3H), 7.35 (m, 2H), 8.50 (d, 1H), 8.60 (d, 1H); MS 343 (M+H)⁺.

### Intermediate 13: 3-Amino-3,4-dihydro-1,6-naphthyridin-2(1H)-one dihydrochloride

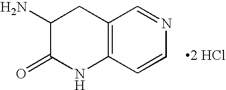

To a stirred solution of tert-butyl(2-oxo-1,2,3,4-tetrahydro-1,6-naphthyridin-3-yl)carbamate (Intermediate 14; 1.24 g, 4.7 mmol) in dioxan (10 ml) was added a 4M solution of HCl in dioxane (60 ml). The reaction was stirred at room temperature for 48 hours then evaporated under reduced pressure to yield a solid. This solid was dried on high vac for 3 hours to yield the product as the dihydrochloride salt (1.2 g, 109%). ¹H NMR: 3.25 (t, 1H), 3.50 (m, 1H), 4.45 (m, 1H), 7.40 (d, 1H), 8.60 (d, 2H), 8.80 (s, 1H), 8.95 (br s, 4H), 12.20 (s, 1H); MS: 164 (M+H)⁺.

### Intermediate 14: tert-Butyl(2-oxo-1,2,3,4-tetrahydro-1,6-naphthyridin-3-yl)carbamate

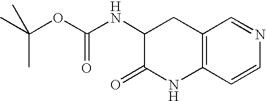

3-[2-(tert-Butoxycarbonylamino)-2-(methoxycarbonyl)ethenyl]4-nitropyridin-1-oxide (Intermediate 15, 1.08 g, 3.18 mmol) was dissolved in ethanol (100 ml) and palladium on carbon catalyst (200 mg, 10% w/w) was added. The mixture was stirred under 1 atmosphere of hydrogen at ambient temperature for 72 hours. After removing the catalyst by filtration through Celite, the filtrate was concentrated under reduced pressure to give a yellow oil which was purified by chromatography on silica gel eluting with 5% methanol in DCM to give the title compound (380 mg, 45%) as a solid.

¹H NMR: 1.4 (s,9H); 3.0 (m,2H); 4.2 (m, 1H); 6.8 (d,1H), 7.0 (bd, 1H); 8.25 (d, 1H); 8.3 (s,1H); 10.5 (s, 1H); MS: 264.

### Intermediate 15: 3-[2-(tert-Butoxycarbonylamino)-2-(methoxycarbonyl)ethenyl]-4-nitropyridine-1-oxide

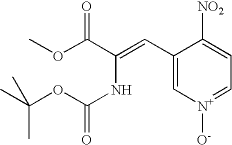

Methyl[(tert-butoxycarbonyl)amino](dimethoxyphosphoryl)acetate (1.633 g, 5.5 mmol) was dissolved in dry THF (30 ml) and cooled to −78 ° C. under nitrogen. Tetramethylguanidine (603 mg., 5.25 mmol) was added and the solution stirred at −78 ° C. for a further 15 mins. A slurry of 4-nitronicotinaldehyde-N-oxide (Eur. J. Med. Chem. 2000, 35(1), 77-82, 850 mg, 5 mmol), in dry THF (5 ml) was added and stirred at −78 ° C. for 3 hours. Water (100 ml) was added and the aqueous phase was extracted with ethyl acetate (3×50 ml). The combined extracts were washed with water (2×20 ml) and brine (20 ml), dried (MgSO₄) and evaporated under reduced pressure to give a yellow oil, which was triturated with ether to give the title compound (1.08 g, 64%) as a yellow solid.

¹H NMR: 1.3 (s, 9H); 3.8 (s, 3H); 7.1 (s, 1H); 8.15 (m, 2H); 8.35 (d, 1H); 8.85 (s, 1H); MS: 338 (M−H)⁺.

### Example 8

### (R)-3-Amino-4-(4-fluorophenyl)-N-(2-oxo-1,2,3,4-tetrahydro-1,6-naphthyridin-3-yl)butanamide

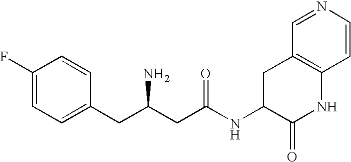

Made using the same procedure as for Example 6 but replacing 3-amino-3,4-dihydro-1,7-naphthyridin2(1H)-one dihydrochloride with 3-amino-3,4-dihydro-1H-1,6-naphthyridin-2-one dihydrochloride.

¹H NMR: 2.10 (m, 1H), 2.20 (m, 1H), 2.55 (m, 1H), 2.65 (m, 1H), 2.90 (t, 1H), 3.10 (m, 1H), 3.20 (m, 1H), 4.55 (m, 1H), 6.80 (m, 1H), 7.10 (t, 2H), 7.20 (m, 2H), 8.25 (m, 2H), 8.40 (m, 1H), 10.60 (br s, 1H); MS: 343 (M+H)⁺.

### Example 9

### (3R)-3-Amino-4-(2-fluorophenyl)-N-(1-methyl-2-oxo-1,2,3,4-tetrahydro-1,5-naphthyridin-3-yl)butanamide dihydrochloride

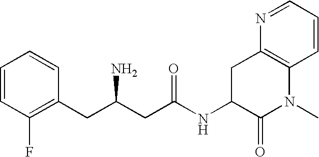

A 4M solution of HCl in dioxan (2.0 ml) was added to tert-butyl{(1R)-1-(2-fluorobenzyl)-3-[(1-methyl-2-oxo-1,2,3,4-tetrahydro-1,5-naphthyridin-3-yl)amino]-3-oxopropyl}carbamate (Intermediate 16; 0.061 g, 0.135 mmol) and the mixture was stirred for 2 hours. The solvent was evaporated under reduced pressure to afford the title compound (0.49 g, 84%). ¹H NMR: 2.13-2.56 (m, 2H), 2.89-3.09 (m, 2H), 3.23-3.33 (m, 2H), 3.27 (s, 3H), 3.65-3.69 (m, 1H), 4.65-4.75 (m, 1H), 7.15-7.21 (m, 2H), 7.31-7.39 (m, 2H), 7.55-7.60 (m, 1H), 7.78 (d, 1H), 8.17 (brs, 2H), 8.28 (d, 1H), 8.68 (t, 1H); MS (+ve ESP): 357 (M+H)⁺.

### Intermediate 16: tert-Butyl{(1R)-1-(2-fluorobenzyl)-3-[(1-methyl-2-oxo-1,2,3,4-tetrahydro-1,5-naphthyridin-3-yl)amino]-3-oxopropyl}carbamate

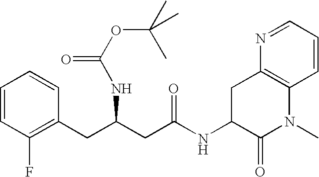

A mixture of tert-butyl[(1R)-3-[(2,5-dioxopyrrolidin-1-yl)oxy]-1-(2-fluorobenzyl)-3-oxopropyl]carbamate (Intermediate 2, 170 mg, 0.44 mmol), 3-amino-1-methyl-3,4-dihydro-1,5-naphthyridin-2(1H)-one hydrochloride (Intermediate 17; 1 in mg, 0.44 mmol) and triethylamine (0.13 ml, 0.98 mmol) in acetonitrile (5 ml) was heated at 100° C. in a microwave for 5 mins. On cooling the suspension was filtered and dried to leave the title compound (43 mg). The filtrate was concentrated and purified on a reverse phase hplc column (5-95% aqueous acetonitrile) to provide the title compound (19 mg). ¹H NMR: 1.27 (s, 9H), 2.35-2.37 (m, 2H), 2.62-2.69 (m, 1H), 2.81-2.92 (m, 1H), 3.08-3.12 (m, 2H), 3.25 (s, 3H), 4.00-4.06 (m, 1H), 4.54-4.66 (m, 1H), 6.63-6.69 (m, 1H), 7.05-7.11 (m, 2H), 7.22-7.33 (m, 3H), 7.48 (d, 1H), 8.15 (brd, 1H), 8.21-8.27 (m, 1H); MS (+ve ESP): 457 (M+H)⁺.

### Intermediate 17: 3-Amino-1-methyl-3,4-dihydro-1,5-naphthyridin-2(1H)-one dihydrochloride

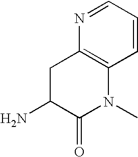

Prepared from Intermediate 18 according to the procedure described in Example 9 to provide the title compound in 100% yield. ¹H NMR: 3.32 (s, 3H), 3.38-3.50 (m, 2H), 4.42-4.46 (m, 1H), 7.49-7.53 (m, 1H), 7.73 (d, 1H), 8.29 (d, 1H), 8.74 (brs, 2H); MS (+ve ESP): 178 (M+H)⁺.

### Intermediate 18: tert-Butyl(1-methyl-2-oxo-1,2,3,4-tetrahydro-1,5-naphthyridin-3-yl)carbamate

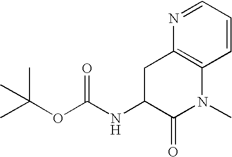

To a suspension of sodium hydride (40 mg, 1 mmol) in DMF (5 ml) at 0 ° C. under nitrogen was added tert-butyl(2-oxo-1,2,3,4-tetrahydro-1,5-naphthyridin-3-yl)carbamate [PCT Int. Appl. (2003), WO 2003074532] (263 mg, 1 mmol) and the reaction mixture was allowed to stir for 30 min at 0° C. Methyl iodide (0.07 ml, 1.1 mmol) was added and the reaction mixture was allowed to stir at ambient temperature for 17 hours. EtOAc (80 ml) was added and the organic phase was washed with brine (3×80 ml), separated and concentrated under reduced pressure to leave crude product. This filtrate was purified on a reverse phase HPLC column (5-95% aqueous acetonitrile) to provide a mixture of the title compound and deprotected material (122 mg), which was used directly in the next stage.

### Example 10

### (3R)-3-Amino-N-[1-(4-fluorobenzyl)-2-oxo-1,2,3,4-tetrahydro-1,5-naphthyridin-3-yl]-4-(2-fluorophenyl)butanamide dihydrochloride

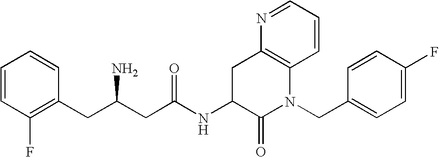

Prepared from Intermediate 19 according to the procedure described in Example 9 to provide the title compound in 84% yield. ¹H NMR: 2.56-2.59 (m, 2H), 2.88-3.08 (m, 2H), 3.29-3.39 (m, 2H), 3.65-3.69 (m, 1H), 4.84-4.97 (m, 1H), 5.07-5.20 (m, 2H) 4H), 7.26-7.45 (m, 5H), 7.58-7.65 (m, 1H), 8.19 (s, 2H), 8.24 (d, 1H), 8.75 (t, 1H); MS (+ve ESP): 451 (M+H)⁺.

### Intermediate 19: tert-Butyl((1R)-1-(2-fluorobenzyl)-3-{[1-(4-fluorobenzyl)-2-oxo-1,2,3,4-tetrahydro-1,5-naphthyridin-3-yl]amino}-3-oxopropyl)carbamate

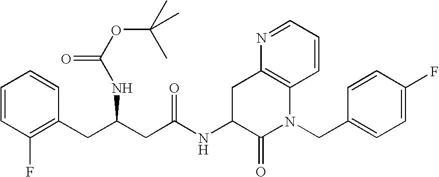

Prepared from Intermediate 20 according to the procedure described for Intermediate 16 to provide the title compound in 62% yield. ¹H NMR: 1.27 (s, 9H), 2.35-2.42 (m, 2H), 2.63-2.71 (m, 1H), 2.82-2.92 (m, 1H), 3.14-3.22 (m, 2H), 3.98-4.11 (m, 1H), 4.74-4.86 (m, 1H), 5.12 (s, 1H), 6.65-6.71 (m, 1H), 7.06-7.14 (m, 4H), 7.18-7.31 (m, 5H), 7.34-7.38 (m, 1H), 8.12 (d, 1H), 8.28-8.36 (m, 1H); MS (+ve ESP): 573 (M+Na)⁺.

### Intermediate 20: 3-Amino-1-(4-fluorobenzyl)-3,4-dihydro-1,5-naphthyridin-2(1H)-one dihydrochloride

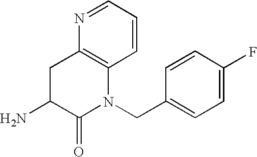

Prepared from Intermediate 21 according to the procedure described for Intermediate 17 to provide the title compound in 100% yield. ¹H NMR: 3.48-3.52 (m, 2H), 4.67-4.75 (m, 1H), 5.19 (s, 2H), 7.13 (t, 2H), 7.31-7.41 (m, 3H), 7.59 (d, 1H), 8.25 (d, 1H), 8.85 (brs, 2H); MS (+ve ESP): 272 (M+H)⁺.

### Intermediate 21: tert-Butyl[1-(4-fluorobenzyl)-2-oxo-1,2,3,4-tetrahydro-1,5-naphthyridin-3-yl]carbamate

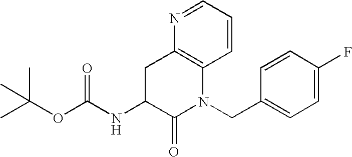

Prepared according to the procedure described for Intermediate 18 by reaction of tert-butyl(2-oxo-1,2,3,4-tetrahydro-1,5-naphthyridin-3-yl)carbamate with 4-fluorobenzyl bromide to provide the title compound in 96% yield. ¹H NMR (: 1.40 (s, 9H), 3.06-3.22 (m, 2H), 4.45-4.54 (m, 1H), 5.11 (s, 2H), 7.08-7.20 (m, 4H), 7.24-7.29 (m, 2H), 7.34 (d, 1H); MS (+ve ESP): 372 (M+H)⁺.

### Example 11

### (3R)-3-Amino-4-(2,5-difluorophenyl)-N-(2-oxo-1,2,3,4-tetrahydro-1,8-naphthyridin-3-yl)butanamide

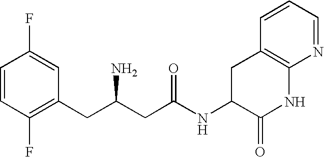

A microwave tube was charged with 3-amino-3,4-dihydro-1,8-naphthyridin-2(1H)-one (Intermediate 5; 44 mg, 0.27 mmol), (3R)-3-[(tert-butoxycarbonyl)amino]-4-(2,5-difluorophenyl)butanoic acid (prepared following the method of Ikemoto et al. J. Amer. Chem. Soc 2004, 126(10), 304; 885 mg, 0.27 mmol), EDAC hydrochloride (52 mg, 0.27 mmol), HOBt (40 mg, 0.3 mmol) and acetonitrile (5 ml). The reaction was heated to 100° C. for 10 minutes then worked up and deprotected (removal of Boc group) using the same procedure as in Example 6 to yield the product (19 mg, 20%). ¹H NMR (CDCl₃): 2.30 (m, 1H), 2.50 (m, 1H), 2.80 (br m, 2H), 3.50 (m, 2H), 4.65 (m, 1H), 7.00 (br m, 4H), 7.50 (d, 1H), 8.20 (m, 1H), 8.25 (br d, 2H); MS 361 (M+H)⁺.

### Example 12

### (3R)-3-Amino-4-(2,5-difluorophenyl)-N-(2-oxo-1,2,3,4-tetrahydro-1,5-naphthyridin-3-yl)butanamide

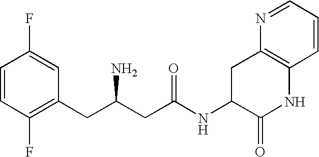

Reaction carried out using the same procedure as for Example 11 but starting with 3-amino-3,4-dihydro-1,5-naphthyridin-2(1H)-one. In this case the intermediate Boc-protected compound was characterised ¹H NMR: 1.27 (s, 9H), 2.37-2.42 (m, 2H), 2.58-2.71 (m, 1H), 2.84-2.94 (m, 1H), 3.05-3.10 (m, 2H), 4.01-4.08 (m, 1H), 4.54-4.67 (m, 1H), 6.71 (t, 1H), 7.04-7.19 (m, 5H), 8.08-8.10 (m, 1H), 8.19-8.26 (m, 1H), 10.36 (s, 1H). MS: 483 (M+Na)⁺. Removal of the protecting group by the same procedure as in Example 11 gave the product:

¹H NMR: 2.56-2.58 (m, 2H), 2.87-3.06 (m, 2H), 3.21-3.28 (m, 2H), 3.68-3.73 (m, 1H), 4.70-4.80 (m, 1H), 7.14-7.29 (m, 3H), 7.51.7.58 (m, 2H), 8.20 (brs, 2H), 8.25 (d, 1H), 8.67 (t, 1H), 10.84 (s, 1H); MS: 361 (M+H)⁺.

### Example 13

### (3R)-3-Amino-4-(2-fluorophenyl)-N-(2-oxo-1,2-dihydroquinolin-3-yl)butanamide hydrochloride

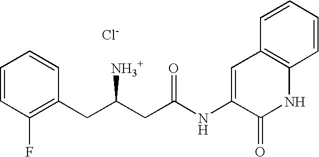

The tert-butyl{(1R)-1-(2-fluorobenzyl)-3-oxo-3-[(2-oxo-1,2-dihydroquinolin-3-yl)amino]propyl}carbamate (Intermediate 22; 100 mg, 0.23 mmol) was treated with a 4M solution of HCl in 1,4-dioxane (5 ml). The mixture was stirred overnight at room temperature. The solvent was evaporated under reduced pressure. The product was triturated in Et₂O and filtered to give the product (81 mg, 91%).

¹H NMR: 2.80-3.16 (4H, m), 7.09-7.21 (4H, m), 7.23-7.41 (4H, m), 8.19 (4H, s), 11.11 (1H, s); MS: 440 (M+)

### Intermediate 22: tert-Butyl{(1R)-1-(2-fluorobenzyl)-3-oxo-3-[(2-oxo-1,2-dihydroquinolin-3-yl)amino]propyl}carbamate

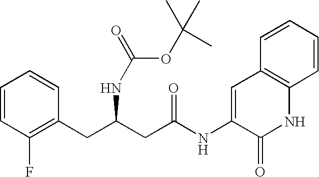

A mixture of (R)-3-tert-butoxycarbonylamino-4-(2-fluorophenyl)butanoic acid (89 mg, 0.3 mmol), 4-methylmorpholine (61 mg, 0.6 mmol) and 3-aminoquinolin-2(1H)-one [C. Juarez-Gordiano et al. Synth Commun 2002, 32 (19), 2959-2963] (48.9 mg, 0.3 mmol) in THF( 5 ml) was treated with DMTMM (83 mg, 0.3 mmol). The mixture was stirred overnight at room temperature. The solvent was evaporated under reduced pressure. The mixture was extracted with Et₂O and washed successively with 1M hydrochloric acid, aqueous sodium bicarbonate and brine. The organic solution was dried (MgSO₄) and concentrated under reduced pressure to give tert-butyl{(1R)-1-(2-fluorobenzyl)-3-oxo-3-[(2-oxo-1,2-dihydroquinolin-3-yl)amino]propyl}carbamate as a solid (104 mg, 79%); MS 462 (M+Na)⁺.

### Example 14

### (3R)-3-Amino-4-(2-fluorophenyl)-N-(5-methoxy-2-oxo-1,2,3,4-tetrahydroquinolin-3-yl)butanamide hydrochloride

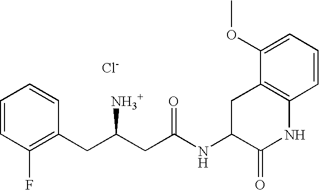

Prepared by the reaction of Intermediate 2 with 3-amino-5-methoxy-3,4-dihydroquinolin-2(1H)-one [US Patent 2004002495] with microwave heating as described for the preparation of Intermediate 4, followed by removal of the Boc protecting group as described for the preparation of Example 1.

¹H NMR: 3.05 (4H, m), 3.68 (1H, m), 3.78 (3H, s), 4.36 (1H, sextet), 6.50 (1H, d), 6.63 (1H, dd), 7.16 (3H, m), 7.35 (2H, m), 8.14 (3H, s), 8.52 (1H, t),10.32 (1H, s); MS 372 (M⁺)

### Example 15

### (3R)-3-Amino-4-(2,5-difluorophenyl)-N-[(3S)-2-oxo-1,2,3,4-tetrahydro-1,8-naphthyridin-3-y]butanamide

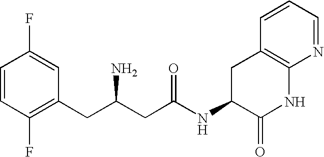

To a solution of (3R)-3-[(tert-butoxycarbonyl)amino]-4-(2,5-difluorophenyl)butanoic acid (0.200 g, 0.634 mmol) in DCM (10 ml) was added HATU (0.265 g, 0.697 mmol), DIPEA (0.122 ml, 0.697 mmol) and (3S)-3-amino-3,4-dihydro-1,8-naphthyridin-2(1H)-one (Intermediate 23, 0.103 g, 0.634 mmol). The reaction mixture was allowed to stir at ambient temperature overnight, the solvent was removed by evaporation and the residue dissolved in hot methanol, cooled and filtered to leave 281 mg of crude product; MS 483 (M+Na)⁺. This was treated with 4M HCl in dioxane (3 ml) and the mixture was allowed to stir at ambient temperature for 3 hours. The solvent was evaporated and the residure purified by reverse phase HPLC 5-95% acetonitrile/water. The product was treated with mp-carbonate to obtain the title compound (0.060 g, 26%). ¹H NMR δ: 1.65 (brs, 2H), 2.13-2.18 (dd, 1H), 2.22-2.28 (m, 1H), 2.59-2.76 (m, 2H), 2.87-2.95 (t, 1H), 3.02-3.09 (m, 1H), 3.27-3.29 (m, 1H), 4.49-4.56 (m, 1H), 6.96-6.99 (m, 1H), 7.05-7.10 (m, 1H), 7.16-7.22 (m, 2H), 7.61 (d, 1H), 8.12 (d, 1H), 8.38 (d, 1H), 10.64 (s, 1H); MS 361 (M+H)⁺.

### Example 16

### (3R)-3-Amino-4-(2,5-difluorophenyl)-N-[(3R)-2-oxo-1,2,3,4-tetrahydro-1,8-naphthyridin-3-yl]butanamide dihydrogen chloride

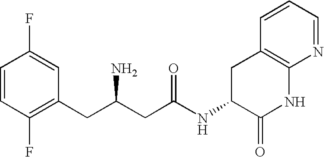

Procedure as above for Example 15 except utilising (3R)-3-amino-3,4-dihydro-1,8-naphthyridin-2(1H)-one (Intermediate 24). The hydrogen chloride salt was obtained in 48% yield by trituration with ether. ¹H NMR δ: 2.65-2.65 (m, 2H), 2.81-3.09 (m, 4H), 3.68-3.73 (m, 1H), 4.46-4.55 (m, 1H), 6.96-7.01 (dd, 1H), 7.12-7.31 (m, 3H), 7.62 (d, 1H), 8.25 (brs, 2H), 8.58 (d, 1H), 10.74 (s, 1H); MS 361 (M+H)³⁰ .

### Intermediate 23: (3S)-3-amino-3,4-dihydro-1,8-naphthyridin-2(MH)-one and

### Intermediate 24: (3R)-3-amino-3,4-dihydro-1,8-naphthyridin-2(1H)-one

The title compounds were prepared by the separation of (rac)-3-amino-3,4-dihydro-1,8-naphthyridin-2(1H)-one Intermediate 5 (6.0 g) by preparative HPLC (2 injections). First enantiomer eluted was (3S)-3-amino-3,4-dihydro-1,8-naphthyridin-2(1H)-one (2.78 g) 88.4% e.e. Second enantiomer eluted was (3R)-3-amino-3,4-dihydro-1,8-naphthyridin-2(1H)-one (3.02 g) 82.7% e.e.

Conditions:

| Instrument | Kronlab | |
|---|---|---|
| Column | Merck 100 mm 20 μm Chiralpak AD | |
| Eluent | MeOH | |
| Oven Temperature | Ambient | |
| Flow | 250 | ml/min |
| Wavelength | 250, 280 | nm |
| Sample Conc | 15 | mg/ml, MeOH |
| Injection volume | 200 | ml (3 g) |
| Run Time | 80 | min |

### Example 17

### (3R)-3-Amino-4-(2,4,5-trifluorophenyl)-N-[(3S)-2-oxo-1,2,3,4-tetrahydro-1,8-naphthyridin-3-yl]butanamide

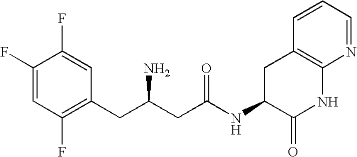

To a solution of (3R)-3-[(tert-butoxycarbonyl)amino]-4-(2,4,5-trifluorophenyl)butanoic acid (1.50 g, 4.50 mmol) in DCM (100 ml) was added HATU (1.88 g, 4.95 mmol), DIPEA (0.864 ml, 4.95 mmol) and (3S)-3-amino-3,4-dihydro-1,8-naphthyridin-2(1H)-one (Intermediate 23, 0.734 g, 4.50 mmol). The reaction mixture was allowed to stir at ambient temperature overnight, the solvent was removed by evaporation and the residue dissolved in boiling methanol, cooled and filtered. The solid was washed sequentially with water (50 ml) and MeOH (50 ml) and then dried in vacuo to leave the Boc protected product, MS 501 (M+Na)⁺as a colourless solid.

This was treated with 4M HCl in dioxane (15 ml) and the mixture was allowed to stir at ambient temperature for 15 hours. The volatiles were removed and the residue taken up in water (20 ml) and 2M NaOH was added until the solution was basic (pH 10). The resulting solid was filtered off and washed twice with water (20 ml) and dried in vacuo to yield the title compound (1.36 g, 80%) as a colourless solid; ¹H NMR δ: 1.81 (s, 2H), 2.14 (dd, 1H), 2.25 (dd, 1H), 2.57 (dd, 1H), 2.70 (dd, 1H), 2.89 (t, 1H), 3.05 (dd, 1H), 3.29 (m, 1H), 4.51 (quintet, 1H), 6.95 (dd, 1H), 7.43 (m, 2H), 7.59 (d, 1H), 8.10 (m, 1H), 8.36 (d, 1H), 10.61 (s, 1H); MS m/z 379 (M+H⁺).

### Example 18

### (3R)-3-Amino-4-(2,4,5-difluorophenyl)-N-[(3R)-2-oxo-1,2,3,4-tetrahydro-1,8-naphthyridin-3-yl]butanamide

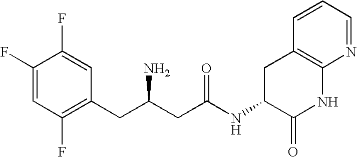

Procedure as for example 17 except utilising (3R)-3-amino-3,4-dihydro-1,8-naphthyridin-2(1H)-one (Intermediate 24), yield 72%.

¹H NMR : 2.14 (dd, 1H), 2.23 (dd, 1H), 2.58 (dd, 1H), 2.67 (dd, 1H), 2.89 (t, 1H), 3.03 (dd, 1H), 3.11-3.41 (m, 3H), 4.52 (quintet, 1H), 6.95 (dd, 1H), 7.35-7.49 (m, 2H), 7.58 (d, 1H), 8.10 (d, 1H), 8.35 (d, 1H), 10.61 (s, 1H); MS: 379 (M+H)⁺.

### Example 19

### (3R)-3-Amino-4-(2-fluorophenyl)-N-(1-methyl-2-oxo-1,2,3,4-tetrahydro-1,8-naphthyridin-3-yl)butanamide

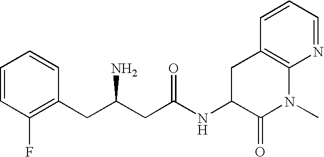

A solution of 3-amino-1-methyl-3,4-dihydro-1,8-naphthyridin-2(1H)-one (Intermediate 25, 95 mg, 0.54 mmol) and tert-butyl[(1R)-3-[(2,5-dioxopyrrolidin-1-yl)oxy]-1-(2-fluorobenzyl)-3-oxopropyl]carbamate (Intermediate 2, 211 mg, 0.54 mmol) in acetonitrile (5 ml) was heated in a microwave at 150° C. for 15 mins. The solvent was evaporated in vacuo to leave a pale brown solid. This solid was triturated with water then filtered and dried under high vac to yield the crude Boc protected product. The solid was suspended in dioxane (5 ml) and treated with 4M HCl in dioxane (15 ml). The resulting reaction was stirred over night at ambient temperature then evaporated in vacuo. The resulting residue was partitioned between 1M NaOH (2 ml) and DCM (30 ml). The organic layer was separated and the aq layer was re-exracted with DCM (2×40 ml). The combined organic layers were evaporated to a residue which was purified by ion exchange chromatography (10 g SCX-2, eluting with MeOH then 1% NH₃/MeOH) to yield the product (152 mg, 79%). ¹H NMR (CDCl₃): 2.3 (m, 1H), 2.50 (m, 1H), 2.80 (brm, 3H), 3.50 (m, 6H), 4.55 (m, 1H), 6.95 (m, 1H), 7.10 (m, 2H), 7.20 (m, 2H), 7.50 (d, 1H), 8.25 (m, 2H); MS (+ve ESP): 357 (M+H)⁺.

### Intermediate 25: (3-Amino-1-methyl-3,4-dihydro-1,8-naphthyridin-2(1H)-one)

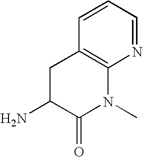

To a stirred solution of tert-butyl(1-methyl-2-oxo-1,2,3,4-tetrahydro-1,8-naphthyridin-3-yl)carbamate (Intermediate 26, 180 mg, 0.65 mmol) in dioxane (4 ml) was added 4M HCl in dioxane (12 ml). The reaction was stirred at ambient temperature for 12 hours then left to stand for 3 days. The solvent was evaporated in vacuo and the resulting residue was partitioned between DCM (30 ml) and 1M NaOH (10 ml). The layers were separated and the aq was re-extracted with DCM (2×30 ml). The combined organic layers were washed with brine (20 ml) then evaporated to an oil. This oil was purified by ion exchange chromatography (10 g SCX-2, MeOH then 1% NH₃) to yield the product as an oil (95 mg, 83%). ¹H NMR (CDCl₃: 2.80 (t, 1H), 3.10 (dd, 1H), 3.50 (m, 3H), 3.60 (dd, 1H), 6.95 (m, 1H), 7.50 (d, 1H), 8.30 (m, 1H); MS (+ve ESP): 178 (M+H)⁺.

### Intermediate 26: (tert-Butyl(1-methyl-2-oxo-1,2,3,4-tetrahydro-1,8-naphthyridin-3-yl)carbamate)

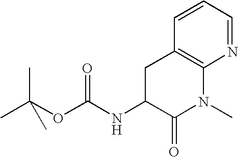

To a stirred solution of tert-Butyl(2-oxo-1,2,3,4-tetrahydro-1,8-naphthyridin-3-yl)carbamate (Intermediate 6, 263 mg, 1 mmol) in anhydrous DMF (5 ml) at 0° C. was added sodium hydride (60% suspension in oil, 42 mg, 1.05 mmol). The reaction was stirred at 0° C. for 30 minutes then treated with MeI (68 μl, 1.1 mmol). The reaction was stirred at 0° C. for 10 minutes then allowed to warm to ambient temperature and stirred for a further 2 hours. A few drops of water were added then the volatiles were evaporated in vacuo. The residue was partitioned between DCM (50 ml) and water (30 ml), the layers were separated and the aqueous layer was re-extracted with DCM (50 ml). The combined organics were washed with brine (30 ml) then dried (MgSO₄), filtered and evaporated to an oil. This oil was purified by column chromatography (20 g Silica, 10% to 40% EtOAc in isohexane) to yield an oil which crystallised on standing (189 mg, 68%). ¹H NMR (CDCl₃): 1.50 (s, 9H), 2.75 (t, 1H), 3.45 (m, 1H), 3.50 (s, 3H), 4.25 (m, 1H), 5.75 (br s, 1H), 6.95 (m, 1H), 7.50 (d, 1H), 8.30 (d, 1H); MS (+ve ESP): 278 (M+H)⁺.

### Example 20

### (3R)-3-Amino-4-(2,5-difluorophenyl)-N-(2-oxo-1,2,3,4-tetrahydro-1,6-naphthyridin-3-yl)butanamide

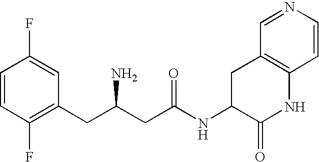

3-Amino-3,4-dihydro-1H-1,6-naphthyridin-2-(1H)-one dihydrochloride (Intermediate 13, 80 mg, 0.34 mmol), (3R)-3-[(tert-butoxycarbonyl)amino]-4-(2,5-difluorophenyl)butanoic acid (107 mg, 0.34 mmol), triethylamine (99 μl, 0.71 mmol), EDAC.HCl (65 mg, 0.34 mmol) and HOBt (50 mg, 0.37 mmol) was suspended in acetonitrile (5 ml). The reaction was heated in a microwave at 100° C. for 12 minutes then the volatiles were removed in vacuo. The resulting pale brown solid was triturated with water then filtered and dried under high vacuum. This solid was dissolved in dioxane (5 ml) and treated with 4M HCl in dioxane (15 ml), the resulting reaction was stirred over night at ambient temperature then evaporated in vacuo. The residue was partitioned between 1M NaOH (2 ml) and DCM (30 ml), the organic layer was separated and the aq was re-extracted with DCM (2×40 ml). The combined organics were evaporated to a residue which was first purified by ion exchange chromatography (10 g SCX-2, MeOH then 1% NH₃/MeOH) then further purified by normal phase chromatography (2 g silica, 5% to 20% MeOH/DCM) to yield the product (10 mg, 8%). ¹H NMR (CD₃OD): 2.35 (m, 1H), 2.45 (m, 1H), 2.85 (m, 2H), 3.05 (m, 1H), 3.20 (m, 1H), 3.55 (m, 1H), 4.70 (m, 1H), 6.90 (d, 1H), 7.00 (m, 1H), 7.10 (m, 2H), 8.25 (m, 2H); MS (+ve ESP): 361 (M+H)⁺.

### Example 21

### (3R)-3-Amino-4-(2,5-difluorophenyl)-N-(2-oxo-1,2,3,4-tetrahydro-1,7-naphthyridin-3-yl)butanamide

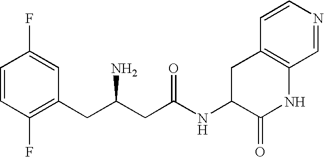

Prepared from Intermediate 9 according to the procedure described for Example 20 to provide the title compound in 31% yield. ¹H NMR: 2.15 (dd, 1H), 2.25 (m, 1H), 2.65 (m, 3H), 2.95 (t, 1H), 3.05 (m, 1H), 4.50 (m, 1H), 7.10 (m, 1H), 7.20 (m, 3H), 8.10 (m, 2H), 8.40 (d, 1H), 10.5 (br s, 1H); MS (+ve ESP): 361 (M+H)⁺.

### Example 22

### (3R)-3-Amino-N-(1-ethyl-2-oxo-1,2,3,4-tetrahydro-18-naphthyridin-3-yl)-4-(2-fluorophenyl)butanamide

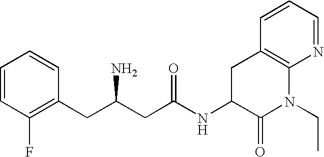

Prepared from Intermediate 27 according to the procedure described for Example 19 to provide the title compound in 55% yield. ¹H NMR: 1.10 (t, 3H), 1.70 (br s, 2H), 2.10 (m, 2H), 2.65 (m, 2H), 2.90 (t, 1H), 3.00 (m, 1H), 3.95 (m, 1H), 4.10 (m, 1H), 4.50 (m, 1H), 7.05 (m, 1H), 7.10 (t, 2H), 7.25 (m, 2H), 7.65 (d, 1H), 8.25 (d, 1H), 8.45 (d, 1H); MS (+ve ESP): 371 (M+H)⁺.

### Intermediate 27: 3-Amino-1-ethyl-3,4-dihydro-1,8-naphthyridin-2(1H)-one dihydrochloride

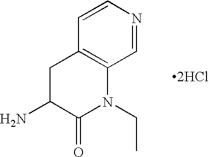

Prepared from Intermediate 28 according to the procedure described for Intermediate 31 to provide the title compound in 100% yield. ¹H NMR: 1.15 (t, 3H), 3.10 (t, 1H), 3.25 (dd, 1H), 4.00 (m, 1H), 4.15 (m, 1H), 4.35 (m, 1H), 7.10 (m, 1H), 7.25 (br s, 2H), 8.75 (d, 1H), 8.30 (d, 1H), 8.80 (br s, 2H); MS (+ve ESP): 192 (M+H)⁺.

### Intermediate 28: tert-Butyl(1-ethyl-2-oxo-1,2,3,4-tetrahydro-1,8-naphthyridin-3-yl)carbamate

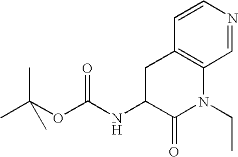

Prepared from Intermediate 6 according to the procedure described for Intermediate 32, replacing the benzyl bromide with ethyl iodide, to provide the title compound in 73% yield. ¹H NMR (CDCl₃): 1.25 (t, 3H), 1.50 (s, 9H), 2.70 (t, 1H), 3.45 (m, 1H), 4.10 (m, 1H), 4.30 (m, 2H), 5.75 (br s, 1H), 6.95 (t, 1H), 7.50 (d, 1H), 8.30 (d, 1H); MS (+ve ESP): 292 (M+H)⁺.

### Example 23

### (3R)-3-Amino-N-[1-(cyclopropylmethyl)-2-oxo-1,2,3,4-tetrahydro-1,8-naphthyridin-3-yl]-4-(2-fluorophenyl)butanamide

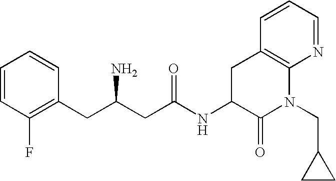

Prepared from Intermediate 29 according to the procedure described for Example 19 to provide the title compound in 51% yield. ¹H NMR: 0.35 (m, 4H), 1.20 (m, 1H), 1.65 (br s, 2H), 2.20 (m, 2H), 2.70 (m, 2H), 2.95 (t, 1H), 3.05 (m, 1H), 3.80 (dd, 1H) 4.05 (m, 1H), 4.55 (m, 1H), 7.00 (m, 1H), 7.10 (t, 2H), 7.30 (m, 2H), 7.65 (d, 1H), 8.25 (d, 1H), 8.50 (m 1); MS (+ve ESP): 397 (M+H)⁺.

### Intermediate 29: 3-Amino-1-(cyclopropylmethyl)-3,4-dihydro-1,8-naphthyridin-2(1H)-one dihydrochloride

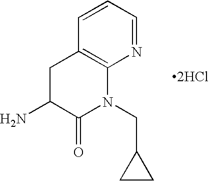

Prepared from Intermediate 30 according to the procedure described for Intermediate 31 to provide the title compound in 100% yield. ¹H NMR: 0.35 (m, 4H), 1.15 (m, 1H), 3.08 (t, 1H), 3.25 (dd, 1H), 3.80 (dd, 1H), 4.02 (dd, 1H), 4.35 (m, 1H), 7.10 (m, 1H), 7.22 (br s, 2H), 7.75 (d, 1H), 8.30 (m, 1H), 8.75 (br s, 2H); MS (+ve ESP): 218 (M+H)⁺.

### Intermediate 30: tert-Butyl[1-(cyclopropylmethyl)-2-oxo-1,2,3,4-tetrahydro-1,8-naphthyridin-3-yl]carbamate

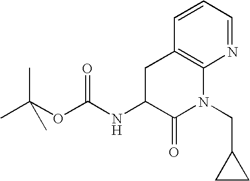

Prepared from Intermediate 6 according to the procedure described for Intermediate 32, replacing the benzyl bromide with (bromomethyl)cyclopropane, to provide the title compound in 73% yield. ¹H NMR (CDCl₃): 0.40 (m, 4H), 0.85 (m, 1H), 1.50 (s, 9H), 2.75 (t, 1H), 3.50 (m, 1H), 3.95 (m, 1H), 4.15 (m, 1H), 4.30 (m, 1H), 5.80 (br s, 1H), 6.95 (m, 1H), 7.50 (d, 1H), 8.25 (d, 1H); MS (+ve ESP): 318 (M+H)⁺.

### Example 24

### (3R)-3-Amino-4-(2,5-difluorophenyl)-N-[1-(4-fluorobenzyl)-2-oxo-1,2,3,4-tetrahydro-1,8-naphthyridin-3-yl]butanamide

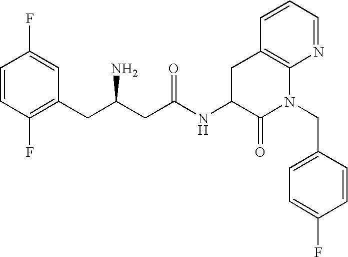

Prepared from 3-amino-1-(4-fluorobenzyl)-3,4-dihydro-1,8-naphthyridin-2(1H)-one di hydrochloride (Intermediate 31) according to the procedure described for Example 20 to provide the title compound in 23% yield. ¹H NMR (CDCl₃): 2.30 (dd, 1H), 2.50 (m, 1H), 2.80 (m, 3H), 3.50 (m, 2H), 4.60 (m, 1H), 5.30 (dd, 2H), 7.00 (m, 6H), 7.40 (m, 2H), 7.50 (d, 1H), 8.10 (m, 1H), 8.30 (d, 1H); MS (+ve ESP): 469 (M+H)⁺.

### Intermediate 31: 3-Amino-1-(4-fluorobenzyl)-3,4-dihydro-1,8-naphthyridin-2(1H)-one di hydrochloride

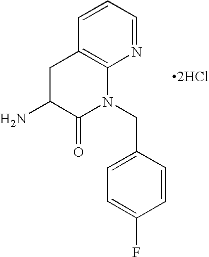

To a stirred solution of tert-butyl[1-(4-fluorobenzyl)-2-oxo-1,2,3,4-tetrahydro-1,8-naphthyridin-3-yl]carbamate (Intermediate 32, 310 mg, 0.84 mmol) in dioxane (5 ml) was added 4M HCl in dioxane (20 ml). The reaction was stirred over night at ambient temperature then evaporated in vacuo to yield the product as the dihydrochloride salt (290 mg, 100%). ¹H NMR: 3.20 (m, 1H), 3.35 (m, 1H), 4.50 (m, 1H), 5.25 (dd, 2H), 6.80 (br s, 1H), 7.10 (m, 3H), 7.35 (m, 2H), 7.80 (d, 1H), 8.25 (d, 1H), 8.85 (br s, 3H); MS (+ve ESP): 272 (M+H)⁺.

### Intermediate 32: tert-Butyl[1-(4-fluorobenzyl)-2-oxo-1,2,3,4-tetrahydro-1,8-naphthyridin-3-yl]carbamate

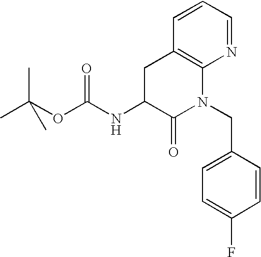

To a stirred solution of tert-Butyl(2-oxo-1,2,3,4-tetrahydro-1,8-naphthyridin-3-yl)carbamate (Intermediate 6, 300 mg, 1.14 mmol) in anhydrous DMF (5 ml) was added sodium hydride (60% suspension in oil, 50 mg, 1.25 mmol). The reaction was stirred at ambient temperature for 10 minutes then treated with 4-fluorobenzyl bromide (149 μl, 1.2 mmol). The reaction was stirred at ambient temperature for 30 minutes then quenched with water (1 ml). The volatiles were evaporated in vacuo and the residue was partitioned between EtOAc (35 ml) and water (5 ml). The layers were separated and the aq was re-extracted with EtOAc (35 ml). The combined organics were washed with brine (10 ml) then dried (MgSO₄), filtered and evaporated to an oil. This oil was purified by column chromatography (20 g Si catridge, eluting with neat isohexane to 20% EtOAc/isohexane gradient) to yield an oil which crystallised on standing (330 mg, 78%). ¹H NMR (CDCl₃): 1.50 (s, 9H), 2.75 (t, 1H), 3.45 (m, 1H), 4.25 (m, 1H), 5.35 (dd, 2H), 5.75 (br s, 1H), 6.95 (m, 3H), 7.40 (m, 2H), 7.50 (d, 1H), 8.30 (d, 1H); MS (+ve ESP): 372 (M+H)⁺.

### Example 25

### (3S)-3-Amino-3-(2,5-difluorophenyl)-N-[1-(4-fluorobenzyl)-2-oxo-1,2,3,4-tetrahydro-1,8-naphthyridin-3-yl]-N-methylpropanamide

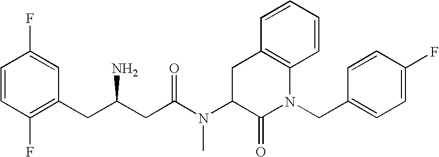

Prepared from Intermediate 33 according to the procedure described for Example 20 to provide the title compound in 16% yield. ¹H NMR: 2.45 (m, 1H), 2.60 (m, 2H),2.80 (m, 4H), 3.00 (d, 3H), 3.30 (q, 1H), 3.60 (m, 1H), 5.30 (m, 2H), 5.50 (m, 1H), 6.95 (m, 7H), 7.40 (m, 3H), 8.25 (d, 1H); MS (+ve ESP): 483 (M+H)⁺.

### Intermediate 33: 1-(4-Fluorobenzyl)-3-(methylamino)-3,4-dihydro-1,8-naphthyridin-2(1H)-one dihydrochloride

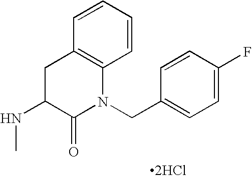

To a stirred solution of tert-butyl[1-(4-fluorobenzyl)-2-oxo-1,2,3,4-tetrahydro-1,8-naphthyridin-3-yl]methylcarbamate (Intermediate 34, 213 mg, 0.55 mmol) in dioxane (10 ml) was added 4M HCl in dioxane (40 ml). The resulting reaction was stirred overnight at ambient temperature then evaporated in vacuo. The resulting solid was triturated with ether then filtered and dried under high vacuum to yield the product as the dihydrochloride salt (188 mg, 95%). ¹H NMR: 2.70 (s, 3H), 3.20 (t, 1H), 3.40 (m, 1H), 4.50 (m, 1H), 5.25 (dd, 2H), 7.10 (m, 3H), 7.35 (m, 2H), 7.80 (d, 1H), 8.30 (d, 1H); MS (+ve ESP): 286 (M+H)⁺.

### Intermediate 34: tert-Butyl[1-(4-fluorobenzyl)-2-oxo-1,2,3,4-tetrahydro-1,8-naphthyridin-3-yl]methylcarbamate

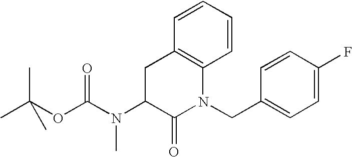

Prepared from Intermediate 32 according to the procedure described for Intermediate 32, replacing the benzylbromide with methyl iodide, to provide the title compound in 90% yield.

¹H NMR (CDCl₃): 1.50 (s, 9H), 2.90 (m, 4H), 3.30 (m, 1H), 5.30 (m, 2H), 6.95 (m, 3H), 7.45 (m, 3H), 8.25 (m, 1H); MS (+ve ESP): 386 (M+H)⁺.

### Example 26

### Methyl[3-1[(3R)-3-amino-4-(2-fluorophenyl)butanoyl]amino}-2-oxo-3,4-dihydroquinolin-1(2H)-yl]acetate

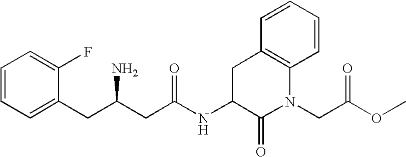

Prepared by deprotection of Intermediate 35 by the method of Example 1, giving a pale yellow solid, 76 mg. ¹H NMR: 2.51-2.64 (m, 8H), 2.85-3.12 (m, 4H), 3.61-3.75 (m, 4H), 4.39-4.51 (m, 1H), 4.56 (d, 1H), 4.80 (dd, 1H), 6.97-7.09 (m, 2H), 7.12-7.42 (m, 6H), 8.25 (s, 3H), 8.60-8.70 (m, 1H); MS (M+H)⁺414.

### Intermediate 35: Methyl[3-{[(3R)-3-[(tert-butoxycarbonyl)amino]-4-(2-fluorophenyl)butanoyl]amino}-2-oxo-3,4-dihydroquinolin-1(2H)-yl]acetate

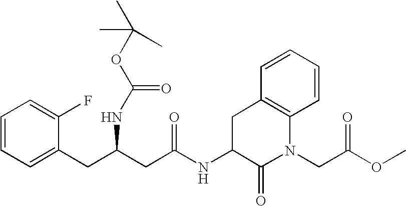

DMTMM (162 mg, 0.59 mmol) was added in one portion to a mixture of (3R)-3-[(tert-butoxycarbonyl)amino]-4-(2-fluorophenyl)butanoic acid (134 mg, 0.45 mmol), methyl (3-amino-2-oxo-3,4-dihydro-2H-quinolin-1-yl)-acetate (106 mg, 0.45 mmol) (CAS no. 599193-11-0; prepared according to the method in WO2003074532), and N-methylmorpholine (0.12 ml, 1.13 mmol) in THF (5 ml). The mixture was stirred overnight at room temperature. The reaction mixture was diluted with DCM and washed successively with 1M HCl and then sodium bicarbonate. The organic solution was concentrated under reduced pressure and the residue was purified by MPLC on silica (Isco Companion®; gradient elution from 100% DCM to 30% ethyl acetate/DCM) to give the title compound as a colourless foam. ¹H NMR: 1.38 (s, 9H), 2.39-2.61 (m, 2H), 2.75-3.02 (m, 3H), 3.50 (in, 1H), 3.76 (s, 3H), 4.19 (m, 1H), 4.50 (dd, 1H), 4.55-4.66 (m, 1H), 4.89 (d, 1H), 6.64-6.75 (1H, m), 6.80 (d, m), 6.94-7.13 (m, 3H), 7.16-7.29 (m, 5H); MS (M+Na)⁺536.

Examples 27-32 were made following the procedure described for Example 9, from Intermediates 36-41 respectively.

### Example 27

### (3R)-3-Amino-4-(2-fluorophenyl)-N-{1-[4-(methylsulfonyl)benzyl]-2-oxo-1,2,3,4-tetrahydro-1,5-naphthyridin-3-yl}butanamide dihydrochloride

### Example 28

### (3R)-3-amino-4-(2-fluorophenyl)-N-{2-oxo-1-[4-(trifluoromethoxy)benzyl]-1,2,3,4-tetrahydro-1,5-naphthyridin-3-yl}butanamide dihydrochloride

### Example 29

### (3R)-3-amino-4-(2-fluorophenyl)-N-{2-oxo-1-[4-(trifluoromethyl)benzyl]-1,2,3,4-tetrahydro-1,5-naphthyridin-3-yl}butanamide dihydrochloride

### Example 30

### (3R)-3-amino-4-(2-fluorophenyl)-N-{2-oxo-1-14-(1,2,3-thiadiazol-4-yl)benzyl]-1,2,3,4-tetrahydro-1,5-naphthyridin-3-yl}butanamide dihydrochloride

### Example 31

### (3R)-3-amino-4-(2-fluorophenyl)-N-{1-[2-(4-methoxyphenyl)ethyl]-2-oxo-1,2,3,4-tetrahydro-1,5-naphthyridin-3-yl}butanamide dihydrochloride

### Example 32

### (3R)-3-amino-4-(2-fluorophenyl)-N-{1-[2-(4-fluorophenyl)ethyl]-2-oxo-1,2,3,4-tetrahydro-1,5-naphthyridin-3-yl}butanamide dihydrochloride

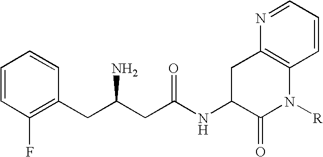

| Eg | R | ¹H NMR | MS |
|---|---|---|---|
| 27 | 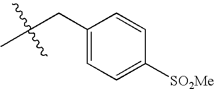 | 2.55-2.61 (m, 2 H), 2.91-3.08 (m, 2 H), 3.17 (s, 3 H), 3.37-3.43 (m, 2 H), 3.70-3.75 (m, 1 H), 4.93-5.04 (m, 1 H), 5.21-5.37 (m, 2 H), 7.17-7.23 (m, 2 H), 7.33-7.41 (m, 1 H), 7.46-7.49 (m, 1 H), 7.51-7.54 (m, 2 H), 7.59-7.62 (dd, 1 H), 7.87 (d, 2 H), 8.21 (brs, 2 H), 8.28 (d, 2 H), 8.76-8.79 (t, 1 H). | 511 (MH)⁺ |
| 28 | 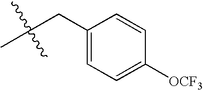 | 2.54-2.57 (m, 2 H), 2.88-2.98 (m, 1 H), 3.02-3.08 (m, 1 H), 3.21-3.26 (m, 2 H), 3.68-3.74 (m, 1 H), 4.79-4.89 (m, 1 H), 5.11-5.24 (m, 2 H), 7.18-7.26 (m, 3 H), 7.30-7.32 (m, 2 H), 7.34-7.40 (m, 5 H), 8.07 (brs, 2 H), 8.16 (d, 1 H), 8.68-8.73 (dd, 1 H). | 517 (MH)⁺ |
| 29 | 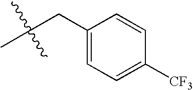 | 2.44-2.46 (m, 2 H), 2.82-2.96 (m, 2 H), 3.19-3.25 (m, 2 H), 3.57-3.60 (m, 1 H), 4.82-4.90 (m, 1 H), 5.18-5.31 (m, 2 H), 7.16-7.21 (m, 3 H), 7.32-7.36 (m, 3 H), 7.47 (d, 2 H), 7.68 (d, 2 H), 8.16 (d, 1 H), 8.68 (t, 1 H). | 501 (MH)⁺ |
| 30 | 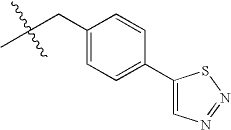 | 2.48-2.50 (m, 2 H), 2.85-2.96 (m, 2 H), 3.14-3.22 (m, 2 H), 3.63-3.66 (m, 1 H), 4.82-4.91 (m, 1 H), 5.19-5.29 (m, 2 H), 7.12 (brs, 2 H), 7.17-7.24 (m, 3 H), 7.31- 7.42 (m, 5 H), 8.07 (d, 2 H), 8.15 (d, 1 H), 8.71 (t, 1 H), 9.57 (s, 1 H). | 517 (MH)⁺ |
| 31 | 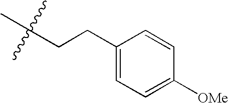 | 2.55-2.57 (m, 2 H), 2.72-2.76 (m, 2 H), 2.88-3.02 (m, 2 H), 3.70 (s, 3 H), 4.03-4.07 (m, 3 H), 4.53-4.64 (m, 1 H), 6.82 (d, 2 H), 7.11 (d, 2 H), 7.17-7.23 (m, 2 H), 7.30-7.35 (m, 3 H), 7.58 (d, 1 H), 8.18 (brs, 2 H), 8.58-8.64 (dd, 1 H). | 477 (MH)⁺ |
| 32 | 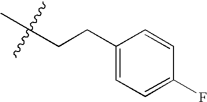 | 2.65-2.73 (m, 2 H), 2.80-2.83 (m, 2 H), 3.03-3.07 (m, 2 H), 4.05-4.09 (m, 3 H), 4.57-4.64 (m, 1 H), 7.06-7.16 (m, 5 H), 7.24-7.31 (m, 4 H), 7.59 (d, 1 H), 8.16 (d, 1 H), 8.46-8.49 (dd, 1 H). | 465 (MH)⁺ |

Intermediates 36-41 were prepared from intermediates 42-47 respectively according to the procedure described for Intermediate 16.

### Intermediate 36: tert-Butyl[(1R)-1-(2-fluorobenzyl)-3-({1-[4-(methylsulfonyl)benzyl]-2-oxo-1,2,3,4-tetrahydro-1,5-naphthyridin-3-yl}amino)-3-oxopropyl]carbamate

### Intermediate 37: tert-Butyl[(1R)-1-(2-fluorobenzyl)-3-oxo-3-({2-oxo-1-[4-(trifluoromethoxy)benzyl]-1,2,3,4-tetrahydro-1,5-naphthyridin-3-yl}amino)propyl]carbamate

### Intermediate 38: tert-Butyl[(1R)-1-(2-fluorobenzyl)-3-oxo-3-({2-oxo-1-[4-(trifluoromethyl)benzyl]-1,2,3,4-tetrahydro-1,5-naphthyridin-3-yl}amino)propyl]carbamate

### Intermediate 39: tert-Butyl[(1R)-1-(2-fluorobenzyl)-3-oxo-3-({2-oxo-1-[4-(1,2,3-thiadiazol-4-yl)benzyl]-1,2,3,4-tetrahydro-1,5-naphthyridin-3-yl}amino)propyl]carbamate

### Intermediate 40: tert-Butyl[(1R)-1-(2-fluorobenzyl)-3-({1-[2-(4-methoxyphenyl)ethyl]-2-oxo-1,2,3,4-tetrahydro-1,5-naphthyridin-3-yl}amino)-3-oxopropyl]carbamate

### Intermediate 41: tert-Butyl[(1R)-1-(2-fluorobenzyl)-3-({1-[2-(4-fluorophenyl)ethyl]-2-oxo-1,2,3,4-tetrahydro-1,5-naphthyridin-3-yl}amino)-3-oxopropyl]carbamate

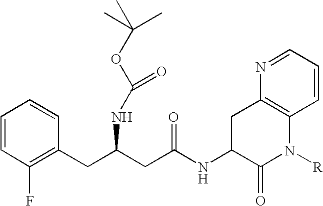

| Inter | R | ¹H NMR | MS |
|---|---|---|---|
| 36 | 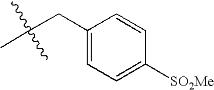 | 1.29 (s, 9 H), 2.37-2.43 (m, 2 H), 2.67-2.72 (m, 1 H), 2.84-2.93 (m, 1 H), 3.18 (s, 3 H), 3.20-3.25 (m, 2 H), 4.05-4.10 (m, 1 H), 4.82-4.93 (m, 1 H), 5.20-5.32 (q, 2 H), 6.66-6.7 1 (m, 1 H), 7.08-7.12 (m, 2 H), 7.20-7.26 (m, 3 H), 7.33-7.36 (m, 1 H), 7.50-7.54 (m, 2 H), 7.86 (d, 2 H), 8.15-8.17 (m, 1 H), 8.32-8.39 (dd, 1 H). | 633 (M + Na) |
| 37 | 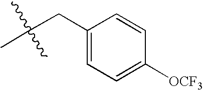 | 1.29 (s, 9 H), 2.32-2.45 (m, 2 H), 2.67-2.72 (m, 1 H), 2.84-2.93 (m, 1 H), 3.18-3.21 (m, 2 H), 4.04-4.12 (m, 1 H), 4.79-4.89 (m, 1 H), 5.13-5.23 (q, 2 H), 6.66-6.71 (t, 1 H), 7.08-7.12 (t, 2 H), 7.21-7.31 (m, 5 H), 7.37- 7.40 (m, 3 H), 8.14-8.16 (m, 1 H), 8.31- 8.37 (dd, 1 H). | 639 (M + Na) |
| 38 | 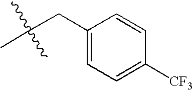 | 1.29 (s, 9 H), 2.33-2.43 (m, 2 H), 2.65-2.72 (m, 1 H), 2.88-2.93 (m, 1 H), 3.19-3.24 (m, 2 H), 4.04-4.12 (m, 1 H), 4.84-4.91 (m, 1 H), 5.19-5.30 (q, 2 H), 6.67 (d, 1 H), 7.08- 7.12 (t, 2 H), 7.20-7.28 (m, 3 H), 7.34-7.37 (dd, 1 H), 7.47 (d, 2 H), 7.68 (d, 2 H), 8.14- 817 (dd, 1 H), 8.32 (d, 1 H). | 623 (M + Na) |
| 39 | 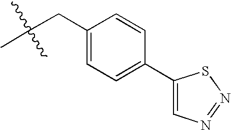 | 1.30 (s, 9 H), 2.38-2.45 (m, 2 H), 2.67-2.73 (m, 1 H), 2.85-2.95 (m, 1 H), 3.18-3.23 (m, 2 H), 4.08-4.13 (m, 1 H), 4.82-4.92 (m, 1 H), 5.25 (s, 2 H), 6.67-6.73 (t, 1 H), 7.08- 7.12 (m, 2 H), 7.21-7.28 (m, 3 H), 7.40- 7.45 (m, 3 H), 8.07 (d, 2 H), 8.14-8.16 (t, 1 H), 8.33-8.40 (dd, 1 H), 9.56 (s, 1 H). | 639 (M + Na) |
| 40 | 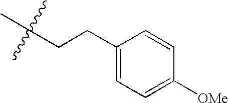 | 1.29 (s, 9 H), 2.31-2.43 (m, 2 H), 2.67-2.76 (m, 3 H), 2.82-2.93 (m, 1 H), 2.98-3.12 (m, 2 H), 3.70 (s, 3 H), 4.03-4.06 (m, 3 H), 4.55-4.65 (m, 1 H), 6.64-6.70 (t, 1 H), 6.62-6.84 (m, 2 H), 7.08-7.15 (m, 4 H), 7.25-7.32 (m, 3 H), 7.58 (d, 1 H), 8.16-8.17 (m, 1 H), 8.20-8.25 (dd, 1 H). | 599 (M + Na) |
| 41 | 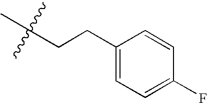 | 1.29 (s, 9 H), 2.32-2.45 (m, 2 H), 2.66-2.71 (m, 1 H), 2.81-3.10 (m, 5 H), 4.03-4.15 (m, 3 H), 4.55-4.65 (m, 1 H), 6.64-6.70 (m, 1 H), 7.05-7.12 (m, 4 H), 7.22-7.31 (m, 5 H), 7.59 (d, 1 H), 8.15-8.17 (m, 1 H), 8.19-8.25 (dd, 1 H). | 587 (M + Na) |

### Intermediate 42: 3-Amino-1-[4-(methylsulfonyl)benzyl]-3,4-dihydro-1,5-naphthyridin-2(1H)-one dihydrochloride

### Intermediate 42: 3-Amino-1-[4-(trifluoromethoxy)benzyl]-3,4-dihydro-1,5-naphthyridin-2(11)-one dihydrochloride

### Intermediate 44: 3-Amino-1-[4-(trifluoromethyl)benzyl]-3,4-dihydro-1,5-naphthyridin-2(1H)-one dihydrochloride

### Intermediate 45: 3-Amino-1-[4-(1,2,3-thiadiazol-4-yl)benzyl]-3,4-dihydro-1,5-naphthyridin-2(1H)-one dihydrochloride

### Intermediate 46: 3-Amino-1-[2-(4-methoxyphenyl)ethyl]-3,4-dihydro-1,5-naphthyridin-2(1H)-one dihydrochloride

### Intermediate 47: 3-Amino-1-[2-(4-fluorophenyl)ethyl]-3,4-dihydro-1,5-naphthyridin-2(1H)-one dihydrochloride

Intermediates 42-47 were prepared from intermediates 48-53 respectively according to the procedure described for Intermediate 16.

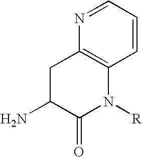

| Inter | R | ¹H NMR | MS |
|---|---|---|---|
| 42 | 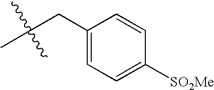 | 3.18 (s, 3 H), 3.49-3.53 (m, 2 H), 4,68-4.75 (m, 1 H), 5.24-5.38 (q, 2 H), 7.33-7.37 (dd, 1 H), 7.50-7.56 (m, 3 H), 7.85 (d, 2 H), 8.24 (d, 1 H), 8.81 (brs, 2 H). | 332 (MH)⁺ |
| 43 | 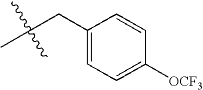 | 3.49 (d, 2 H), 4.61-4.69 (m, 1 H), 5.16-5.29 (q, 2 H), 7.29-7.43 (m, 5 H), 7.54 (d, 1 H), 8.23 (d, 1 H), 8.80 (brs, 2 H). | 338 (MH)⁺ |
| 44 | 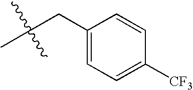 | 3.48 (d, 2 H), 4.70-4.77 (m, 1 H), 5.23-5.37 (q, 2 H), 7.32-7.36 (dd, 1 H), 7.51 (d, 3 H), 7.68 (d, 2 H), 8.24 (d, 1 H), 8.80 (brs, 2 H). | 322 (MH)⁺ |
| 45 | 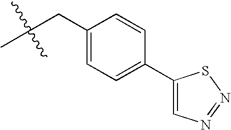 | 3.53 (d, 2 H), 4.70-4.78 (m, 1 H), 5.23-5.35 (t, 2 H), 7.35-7.40 (dd, 1 H), 7.46 (d, 2 H), 7.58 (d, 1 H), 8.07 (d, 2 H), 8.24 (d, 1 H), 8.84 (brs, 2 H), 9.59 (s, 1 H) | 338 (MH)⁺ |
| 46 | 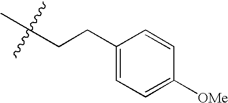 | 2.77-2.80 (t, 2 H), 3.26-3.33 (t, 1 H), 3.39- 3.44 (dd, 1 H), 3.71 (s, 3 H), 4.08-4.13 (m, 2 H), 4.43-4.47 (m, 1 H), 6.84 (d, 2 H), 7.16 (d, 2 H), 7.42-7.45 (m, 1 H), 7.75 (d, 1 H), 8.26 (d, 1 H), 8.72 (brs, 2 H). | 298 (MH)⁺ |
| 47 | 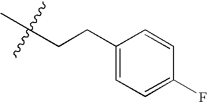 | 2.82-2.86 (t, 2 H), 3.23-3.31 (t, 1 H), 3.37- 3.42 (dd, 1 H), 4.12-4.15 (t, 2 H), 4.43-4.46 (m, 1 H), 7.06-7.11 (t, 2 H), 7.26-7.29 (m, 2 H), 7.39-7.42 (m, 1 H), 7.74 (d, 1 H), 8.25-8.27 (m, 1 H), 8.72 (brs, 2 H). | 286 (MH)⁺ |

### Intermediate 48: tert-Butyl{1-[4-(methylsulfonyl)benzyl]-2-oxo-1,2,3,4-tetrahydro-1,5-naphthyridin-3-yl}carbamate

### Intermediate 49: tert-Butyl{2-oxo-1-[4-(trifluoromethoxy)benzyl]-1,2,3,4-tetrahydro-1,5-naphthyridin-3-yl}carbamate

### Intermediate 50: tert-Butyl{2-oxo-1-[4-(trifluoromethyl)benzyl]-1,2,3,4-tetrahydro-1,5-naphthyridin-3-yl}carbamate

### Intermediate 51: tert-Butyl{2-oxo-1-[4-(1,2,3-thiadiazol-4-yl)benzyl]-1,2,3,4-tetrahydro-1,5-naphthyridin-3-yl}carbamate

### Intermediate 52: tert-Butyl{1-[2-(4-methoxyphenyl)ethyl]-2-oxo-1,2,3,4-tetrahydro-1,5-naphthyridin-3-yl}carbamate

### Intermediate 53: tert-Butyl{1-[2-(4-fluorophenyl)ethyl]-2-oxo-1,2,3,4-tetrahydro-1,5-naphthyridin-3-yl}carbamate

Intermediates 48-53 were prepared according to the procedure described for Intermediate 18 by reaction of tert-butyl(2-oxo-1,2,3,4-tetrahydro-1,5-naphthyridin-3-yl)carbamate with the relevant commercially available benzyl bromide.

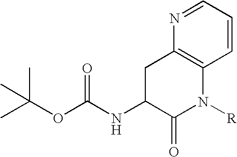

| Inter | R | ¹H NMR | MS |
|---|---|---|---|
| 48 | 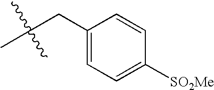 | 1.42 (s, 9 H), 3.12-3.15 (m, 2 H), 3.18 (s, 3 H), 4.54-4.61 (m, 1 H), 5.18-5.32 (q, 2 H), 7.18 (s, 1 H), 7.21-7.24 (m, 1 H), 7.34 (d, 1 H), 7.51 (d, 2 H), 7.87 (d, 2 H), 8.16 (d, 1 H). | 454 (M + Na) |
| 49 | 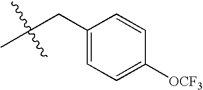 | 1.42 (s, 9 H), 3.12-3.18 (dd, 1 H), 3.26-3.33 (t, 1 H), 4.51-4.56 (m, 1 H), 5.11-5.24 (q, 2 H), 7.17-7.19 (brd, 1 H), 7.22-7.25 (dd, 1 H), 7.30-7.32 (dd, 2 H), 7.36-7.38 (d, 3 H), 8.15 (d, 1 H). | 460 (M + Na) |
| 51 | 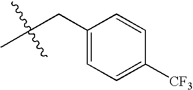 | 1.42 (s, 9 H), 3.12-3.18 (dd, 1 H), 3.27-3.30 (m, 1 H), 4.54-4.57 (m, 1 H), 5.17-5.31 (q, 2 H), 7.20-7.22 (m, 2 H), 7.32 (d, 1 H), 7.46 (d, 2 H), 7.68 (d, 2 H), 8.14 (d, 1 H). | 444 (M + Na) |
| 51 | 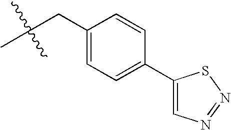 | 1.43 (s, 9 H), 3.13 (dd, 1 H), 3.28-3.30 (m, 1 H), 4.53-4.58 (m, 1 H), 5.23 (s, 2 H), 7.20-7.23 (m, 2 H), 7.38-7.43 (m, 3 H), 8.07 (d, 2 H), 8.13 (d, 1 H), 9.56 (s, 1 H). | 460 (M + Na) |
| 52 | 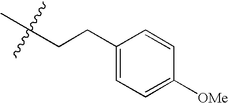 | | 420 (M + Na) r.t 2.18 |
| 53 | 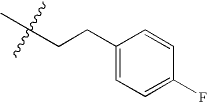 | | 408 (M + Na r.t 2.26 |

### Example 33

### (3R)-3-Amino-4-(2-fluorophenyl)-N-methyl-N-(1-methyl-2-oxo-1,2,3,4-tetrahydro-1,5-naphthyridin-3-yl)butanamide dihydrochloride salt

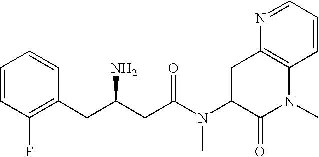

Prepared from Intermediate 54 according to the procedure described in Example 9 to provide the title compound in 83% yield. ¹H NMR: 2.71-2.81 (m, 3H), 2.91 (s, 3H), 3.00-3.19 (m, 2H), 3.26 (s, 3H), 3.63-3.73 (m, 2H), 5.25-5.37 (m, 1H), 7.13-7.21 (m, 2H), 7.28-7.42 (m, 2H), 7.53-7.57 (m, 11H), 7.69-7.75 (m, 11H), 8.10 (brs, 2H), 8.27 (d, 1H)); MS: 371 (M+H)⁺.

### Intermediate 54: tert-Butyl{(1R)-1-(2-fluorobenzyl)-3-[methyl(1-methyl-2-oxo-1,2,3,4-tetrahydro-1,5-naphthyridin-3-yl)amino]-3-oxopropyl}carbamate

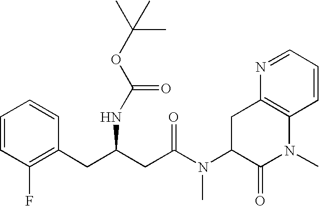

Prepared from Intermediate 55 according to the procedure described for Intermediate 16. This mixture was purified on a reverse phase HPLC column (5-95% aqueous acetonitrile) to provide a mixture of the title compound and deprotected material (122 mg), which was used directly in the next stage.

### Intermediate 55: 1-Methyl-3-(methylamino)-3,4-dihydro-1,5-naphthyridin-2(1H)-one dihydrochloride

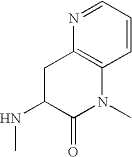

Prepared from Intermediate 56 according to the procedure described in Example 9 to provide the title compound in 100% yield. MS: 192 (M+H)⁺.

### Intermediate 56: tert-Butyl methyl(1-methyl-2-oxo-1,2,3,4-tetrahydro-1,5-naphthyridin-3-yl)carbamate

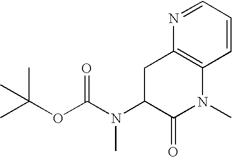

To a suspension of sodium hydride (98 mg, 2.27 mmol) in DMF (5 ml) at 0° C. under nitrogen was added tert-butyl(2-oxo-1,2,3,4-tetrahydro-1,5-naphthyridin-3-yl)carbamate (200 mg, 0.759 mmol) in three portions and the reaction mixture was allowed to stir for 30 min at 0 ° C. Methyl iodide (0.1 ml, 1.66 mmol) was added and the reaction mixture was allowed to stir at ambient temperature for 2.5 hours. EtOAc (80 ml) was added and the organic phase was washed with brine (3×80 ml), separated and concentrated under reduced pressure to leave crude product. This filtrate was purified on a reverse phase HPLC column (5-95% aqueous acetonitrile) to provide the title compound (204 mg, 92%). ¹H NMR (CDCl₃)δ: 1.48 (s, 9H), 2.93 (s, 3H), 3.13-3.23 (m, 1H), 3.34 (s, 3H), 3.45-3.55 (m, 1H), 4.99-5.11 (m, 1H), 7.16-7.21 (m, 2H), 8.21-8.22 (m, 1H).

Examples 34-40 were made by the following procedure from commercially available benzyl chlorides.

To a suspension of sodium hydride (15 mg, 0.63 mmol) in DMF (5 ml) at ambient temperature under nitrogen was added tert-butyl(2-oxo-1,2,3,4-tetrahydro-1,5-naphthyridin-3-yl)carbamate (100 mg, 0.38 mmol) and the reaction mixture was allowed to stir for 30 min at ambient temperature. The appropriate benzyl chloride (0.45 mmol) was added followed by a catalytic amount of potassium iodide and the reaction mixture was allowed to stir at ambient temperature for 17 hours. EtOAc (20 ml) was added and the organic phase was washed with brine (3×80 ml), separated and concentrated under reduced pressure to leave crude product which was taken directly through to the next stage.

The crude products were dissolved in HCl/dioxane (3 ml, 6 mmol) and stirred for three hours. The solvent was removed under vacuum to give crude products which were taken directly through to the next stage.

(3R)-3-[(tert-Butoxycarbonyl)amino]-4-(2-fluorophenyl)butanoic acid (178 mg, 0.6 mmol) was dissolved in dichloromethane and HOBT (81.1 mg, 0.6 mmol), DIPEA (105 μl, 0.6 mmol) and EDCI (115 mg, 0.6 mmol) were added. This solution was added to a solution of the amine, DIPEA (2 eq) in DCM and the reaction mixture was left to stir at ambient temperature for three hours. The dichloromethane was removed in vacuum to leave crude products were taken directly into the next step.

The crude products were treated with HCl/dioxane (3 ml, 6 mmol) and stirred at ambient temperature for two hours. The dioxane was removed under vacuum and the resulting solids purified by reverse phase HPLC column (5-95% aqueous acetonitrile) to provide the title compounds.

Products were analysed on a Waters 2795 Separation Module HPLC MicromassZMD mass spectrometer at ambient temperature, on a Phenomenex Synergi C18 Max-RP 4 um 80 A (50×2.0 mm) column using MeCN/H₂O & 0.02% TFA solvent at 1.10 ml/min.

### Example 34

### (3R)-3-Amino-4-(2-fluorophenyl)-N-[1-(4-methylbenzyl)-2-oxo-1,2,3,4-tetrahydro-1,5-naphthyridin-3-yl]butanamide dihydrochloride

### Example 35

### (3R)-3-Amino-N-[1-(3,4-difluorobenzyl)-2-oxo-1,2,3,4-tetrahydro-1,5-naphthyridin-3-yl]-4-(2-fluorophenyl)butanamide dihydrochloride

### Example 36

### Methyl 3-{[3-{[(3R)-3-amino-4-(2-fluorophenyl)butanoyl]amino}-2-oxo-3,4-dihydro-1,5-naphthyridin-1(2H)-yl]methyl}benzoate dihydrochloride

### Example 37

### 4-{[3-{[(3R)-3-Amino-4-(2-fluorophenyl)butanoyl]amino}-2-oxo-3,4-dihydro-1,5-naphthyridin-1(2H)-yl]methyl}phenyl acetate dihydrochloride

### Example 38

### (3R)-3-amino-N-[1-(3-chloro-4-methoxybenzyl)-2-oxo-1,2,3,4-tetrahydro-1,5-naphthyridin-3-yl]-4-(2-fluorophenyl)butanamide dihydrochloride

### Example 39

### (3R)-3-amino-N-[1-(4-benzoylbenzyl)-2-oxo-1,2,3,4-tetrahydro-1,5-naphthyridin-3-yl]-4-(2-fluorophenyl)butanamide dihydrochloride

### Example 40

### (3R)-N-{1-[4-(acetylamino)benzyl]-2-oxo-1,2,3,4-tetrahydro-1,5-naphthyridin-3-yl}-3-amino-4-(2-fluorophenyl)butanamide dihydrochloride

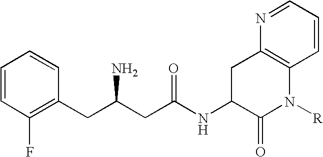

| | | | Retention |
|---|---|---|---|
| | | | time |
| Eg | R | MS | (mins) |
| 34 | 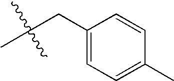 | 469 (MH)⁺ | 1.31 |
| 35 | 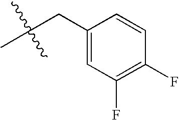 | 447 (MH)⁺ | 2.24 |
| 36 | 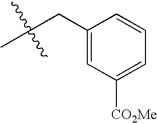 | 489 (MH)⁺ | 2.08 |
| 37 | 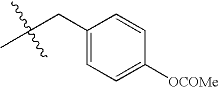 | 449 (MH)⁺ | 1.06 |
| 38 | 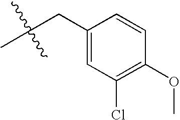 | 497 (MH)⁺ | 1.36 |
| 39 | 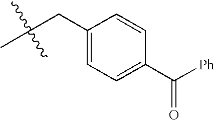 | 537 (MH)⁺ | 1.48 |
| 40 | 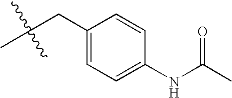 | 490 (MH)⁺ | 1.05 |

Examples 41-46 were made using the same procedure as for Examples 34-40, replacing tert-butyl(2-oxo-1,2,3,4-tetrahydro-1,5-naphthyridin-3-yl)carbamate with tert-butyl(2-oxo-1,2,3,4-tetrahydro-1,8-naphthyridin-3-yl)carbamate

### Example 41

### (3R)-3-amino-4-(2-fluorophenyl)-N-[1-(4-nitrobenzyl)-2-oxo-1,2,3,4-tetrahydro-1,8-naphthyridin-3-yl]butanamide

### Example 42

### (3R)-3-amino-N-[1-(3,4-difluorobenzyl)-2-oxo-1,2,3,4-tetrahydro-1,8-naphthyridin-3-yl]-4-(2-fluorophenyl)butanamide

### Example 43

### methyl 4-{[3-{[(3R)-3-amino-4-(2-fluorophenyl)butanoyl]amino}-2-oxo-3,4-dihydro-1,8-naphthyridin-1(2H)-yl]methyl}benzoate

### Example 44

### (3R)-3-amino-N-[1-(3-chloro-4-methoxybenzyl)-2-oxo-1,2,3,4-tetrahydro-1,8-naphthyridin-3-yl]-4-(2-fluorophenyl)butanamide

### Example 45

### (3R)-3-amino-N-[1-(4-benzoylbenzyl)-2-oxo-1,2,3,4-tetrahydro-1,8-naphthyridin-3-yl]-4-(2-fluorophenyl)butanamide

### Example 46

### (3R)-N-{1-[4-(acetylamino)benzyl]-2-oxo-1,2,3,4-tetrahydro-1,8-naphthyridin-3-yl}-3-amino-4-(2-fluorophenyl)butanamide

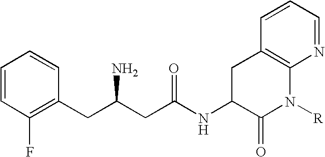

| | | | Retention |
|---|---|---|---|
| Eg | R | MS | time |
| 41 | 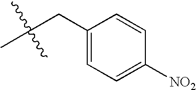 | 478 (MH)⁺ | 1.36 |
| 42 | 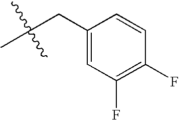 | 469 (MH)⁺ | 1.44 |
| 43 | 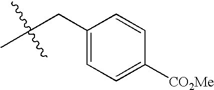 | 491 (MH)⁺ | 1.36 |
| 44 | 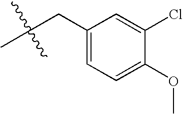 | 497 (MH)⁺ | 1.44 |
| 45 | 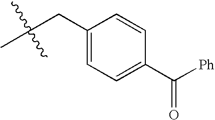 | 537 (MH)⁺ | 1.55 |
| 46 | 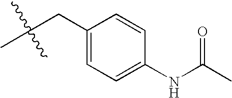 | 490 (MH)⁺ | 1.17 |

Examples 47-50 were prepared from intermediates 57-60 by the method of Example 1

### Example 47

### (3R)-3-Amino-N-(6-fluoro-2-oxo-1,2,3,4-tetrahysroquinolin-3-yl)-4-(2-fluorophenyl)butanamide monohydrochloride

### Example 48

### (3R)-3-Amino-N-(6-methoxy-2-oxo-1,2,3,4-tetrahysroquinolin-3-yl)-4-(2-fluorophenyl)butanamide

### Example 49

### (3R)-3-Amino-4-(2-fluorophenyl)-N-(5-methyl-2-oxo-1,2,3,4-tetrahysroquinolin-3-yl)butanamide monohydrochloride

### Example 50

### (3R)-3-Amino-4-(2,5-difluorophenyl)-N-(5-methoxy-2-oxo-1,2,3,4-tetrahysroquinolin-3-yl)butanamide

| Ex | Structure | ¹H NMR | MS |
|---|---|---|---|
| 47 | 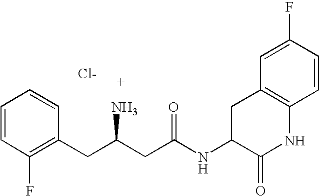 | 2.55 (m, 1 H), 2.65 (m, 1 H), 2.95 (m, 3 H), 3.12 (m, 1 H), 3.70 (m, 1 H), 4.41 (m, 1 H), 6.91 (dd, 1 H), 7.01 (m, 1 H), 7.09 (t, 1 H), 7.20 (m, 2 H), 7.38 (m, 2 H), 8.40 (broad, 3 H), 8.59 (dd, 1 H), 10.40 (s, 1 H) | MH⁺ 360 |
| 48 | 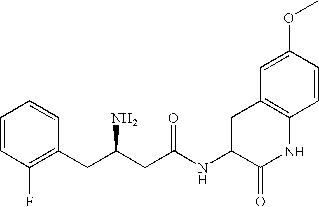 | 2.10 (m, 3 H), 2.60 (m, 2 H), 2.78 (m, 1 H), 2.90 (m, 1 H), 3.20 (broad, 2 H), 3.62 (s, 3 H), 4.32 (m, 1 H), 6.70 (m, 3 H), 7.05 (m, 2 H), 7.19 (m, 2 H), 8.27 (d, 1 H), 10.03 (s, 1 H) | MH⁺ 372 |
| 49 | 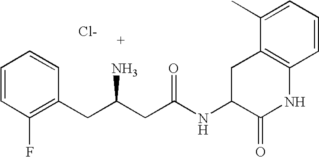 | 2.23 (s, 3 H), 2.57 (m, 1 H), 2.66 (m, 1 H), 2.93 (m, 2 H), 3.08 (m, 12 H), 3.72 (m, 1 H), 4.42 (m, 1 H), 6.73 (d, 1 H), 6.83 (m, 1 H), 7.05 (m, 1 H), 7.21 (m, 2 H), 7.38 (m, 2 H), 8.25 (broad, 3 H), 8.58 (dd, 1 H), 10.28 (s, 1 H) | MH⁺ 356 |
| 50 | 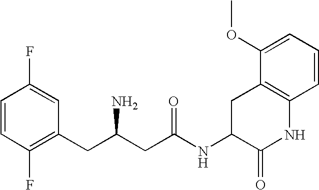 | 2.18 (m, 1 H), 2.28 (m, 1 H), 2.78 (m, 3 H), 2.80 (broad, 2 H), 3.29 (s, 3 H), 4.40 (m, 1 H), 6.51 (d, 1 H), 6.12 (d, 1 H), 7.10 (m, 2 H), 7.19 (m, 2 H), 8.39 (dd, 1 H), 10.25 (s, 1 H) | MH⁺ 390 |

Intermediates 57-59 were prepared by the method given for the preparation of Intermediate 1, utilising the appropriately substituted aminodihydroquinolone.

Intermediate 57: tert-Butyl(1R)-1-(2-fluorobenzyl)-3-[(6-fluoro-2-oxo-1,2,3,4-tetrahydroquinolin-3-yl)amino]-3-oxopropylcarbamate from 3-amino-6-fluoro-3,4-dihydro-2(1H)-quinolinone monohydrochloride (CAS Reg. No: 82420-54-0; WO 2003 074532)

Intermediate 58: tert-Butyl(1R)-1-(2-fluorobenzyl)-3-[(6-methoxy-2-oxo-1,2,3,4-tetrahydroquinolin-3-yl)amino]-3-oxopropylcarbamate from 3-amino-3,4-dihydro-6-methoxy-2(1H)-quinolinone (CAS Reg No: 756756-10-2; WO 2003 074532)

Intermediate 59: tert-Butyl(1R)1-(2-fluorobenzyl)-3-[(5-methyl-2-oxo-1,2,3,4-tetrahydroquinolin-3-yl)amino]-3-oxopropylcarbamate from 3-amino-3,4-dihydro-5-methyl-2(1H)-quinolinone monohydrochloride, intermediate 61

| Inter | Structure | ¹H NMR | MS |
|---|---|---|---|
| 57 | 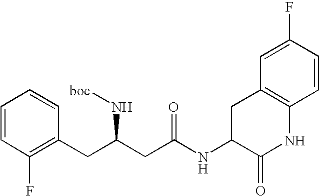 | 1.30 (9 H, s), 2.39 (m, 2 H), 2.67 (m, 1 H), 2.90 (m, 1 H), 3.25 (m, 2 H), 4.08 (m, 1 H), 4.40 (m, 1 H), 6.51 (d, 1 H), 6.14 (d, 1 H), 6.71 (dd, 1 H), 7.11 (m, 4 H), 8.18 (dd, 1 H), 10.28 (s, 1 H) | MNa⁺ 512 |
| 58 | 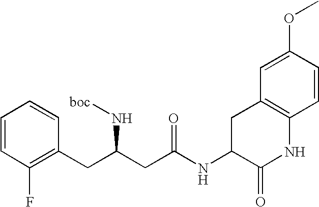 | 1.30 (9 H, s), 2.37 (m, 2 H), 2.69 (m, 1 H), 2.83 (m, 2 H), 3.00 (m, 1 H), 3.71 (s, 3 H), 4.07 (m, 1 H), 4.41 (m, 1 H), 6.68 (m, 1 H), 6.77 (m, 3 H), 7.12 (m, 3 H), 7.27 (m, 3 H), 8.13 (dd, 1 H), 10.14 (s, 1 H) | MNa⁺ 494 |
| 59 | 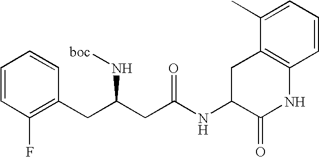 | 1.30 (9 H, s), 2.21 (s, 3 H), 2.39 (m, 2 H), 2.76 (m, 1 H), 2.85 (m, 2 H), 3.09 (dd, 1 H), 4.07 (m, 1 H), 4.42 (m, 1 H), 6.68 (m, 1 H), 6.71 (m, 1 H), 6.80 (m, 1 H), 7.09 (m, 3 H), 7.25 (m, 2 H), 8.15 (dd, 1 H), 10.22 (s, 1 H) | MNa⁺ 478 |

### Intermediate 60: tert-Butyl(1R)-1-(2,5-difluorobenzyl)-3-[(5-methoxy-2-oxo-1,2,3,4-tetrahydroquinolin-3-yl)amino]-3-oxopropylcarbamate

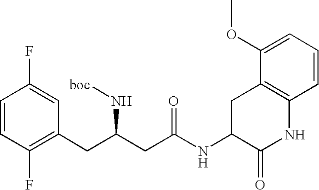

(3R)-3-[(tert-Butoxycarbonyl)amino]-4-(2,5-difluorophenyl)butanoic acid (0.55 mmol, 173 mg) dissolved in DMA (5 ml) was treated with HBTU (0.55 mmol, 209 mg), 3-amino-3,4-dihydro-5-methoxy-2(1H)-quinolinone monohydrochloride (CAS Reg No: 639478-57-2, U.S. Patent application 2004002495) (1.0 mmol, 228 mg) and DIPEA (1.65 mmol, 213 mg). Stirred at ambient temperature for 2.5 hrs. The precipitated solid was filtered off, washed with ether and dried in vacuo to give the title compound (183 mg, 68%) as an amorphous solid.

¹H NMR: 1.30 (9H, s), 2.39 (m, 2H), 2.67 (m, 1H), 2.90 (m, 1H), 3.25 (m, 2H), 4.08 (m, 1H), 4.40 (m, 1H), 6.51 (d, 1H), 6.14 (d, 1H), 66.71 (dd, 1H), 7.11 (m, 4H), 8.18 (dd, 1H), 10.28 (s, 1H); MS (M+Na)⁺512.

### Intermediate 61: 3-Amino-5-methyl-3,4-dihydroquinolin-2(1H)-one monohydrochloride

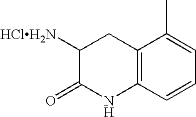

tert-Butyl(5-methyl-2-oxo-1,2,3,4-tetrahydroquinolin-3-yl)carbamate (0.5 mmol, 138 mg) (Intermediate 62) was dissolved in 1,4-dioxane (5 ml) and treated with 4M HCl solution in 1,4-dioxane (1 ml). Stirred at ambient temperature until reaction complete. The precipitated solid was collected and washed with anhydrous ether. Dried under vacuum to yield the product as an amorphous solid (125 mg, 99%)

¹H NMR: 2.23 (s, 3H), 2.87 (t, 1H), 3.31 (dd, 1H), 4.14 (m, 1H), 6.79 (d, 1H), 6.88 (d, 1H), 7.10 (m, 1H), 8.66 (broad, 3H), 10.64 (s, 1H); MS (M+H)⁺177.

### Intermediate 62: tert-Butyl(5-methyl-2-oxo-1,2,3,4-tetrahydroquinolin-3-yl)carbamate

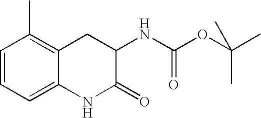

2-Methyl-6-nitrophenylalanine hydrochloride (Intermediate 63; 4.52 g, 17.38 mmol) was dissolved in methanol (30 ml) and water (30 ml) and hydrogenated under an atmosphere of hydrogen using 10% Pd/C (1 g) as catalyst. After 2 hours the slurry was filtered through celite, washed with methanol (50 ml) and water (50 ml) and concentrated under vacuum to give a pink solid. The solid was suspended in dichloromethane (100 ml) and triethylamine (2.67 ml, 19.12 mmol) added followed by di-tert-butyl dicarbonate (3.79 g, 17.38 mmol). After 18 hours the solution was diluted with dichloromethane (100 ml) and washed with water (3×10 ml) and brine (10 ml), dried (sodium sulfate) and evaporated to give a hazy red gum. The crude material was purified by flash column chromatography on silica gel with an eluent gradient of dichloromethane to ethyl acetate to give tert-butyl(5-methyl-2-oxo-1,2,3,4-tetrahydroquinolin-3-yl)carbamate (Intermediate 62) as an off white solid (0.53 g, 11%).

¹H NMR: 10.18 (1H, s), 7.10 (1H, dd), 7.02 (1H, d), 6.86 (1H, d), 6.76 (1H, d), 4.24-4.13 (1H, m), 3.10 (1H, dd), 2.79 (1H, t), 2.27 (3H, s), 1.47 (9H, s); MS 277 (M+H)⁺

### Intermediate 63: 2-Methyl-6-nitrophenylalanine hydrochloride salt

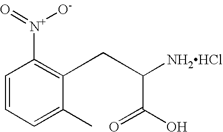

A suspension of diethyl (acetylamino)(2-methyl-6-nitrobenzyl)malonate (Intermediate 64; 7.55 g, 20.63 mmol) in concentrated hydrochloric acid (100 ml) was heated to 100° C. for 8 hours. After cooling to room temperature the mixture was evaporated to dryness then azeotroped with toluene (100 ml) to give the title compound as a yellow solid (4.52 g, 84%). MS 225.25 (M+H)⁺

### Intermediate 64: Diethyl(acetylamino)(2-methyl-6-nitrobenzyl)malonate

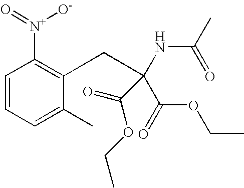

A solution of diethyl acetamidomalonate (3.72 g, 17.12 mmol) was added to a suspension of sodium hydride 60% dispersion (685 mg, 17.12 mmol) in DMF (60 ml) at 0° C. The mixture was allowed to warm to room temperature then cooled to 0° C. 2-Methyl-6-nitrobenzyl bromide (Makosza, M. et al, Tetrahedron, 1984, 40, 1863-1868) (4.33 g, 18.83 mmol) in DMF (25 ml) was then added and the reaction mixture allowed to warm to room temperature and stirred for 18 hours. The solution was diluted with ethyl acetate (100 ml) and washed with water (3×10 ml) and brine (10 ml), dried (MgSO₄) and evaporated to give a dark, clear oil. The crude material was purified by flash column chromatography on silica gel with an eluent gradient of dichloromethane to diethyl ether to give the title compound as a yellow solid.

¹H NMR: 8.08 (1H, s), 7.63 (1H, d), 7.50 (1H, d), 7.37 (1H, dd), 4.13-3.97 (4H, m), 3.94 (2H, s), 2.33 (3H, s), 1.83 (3H, s), 1.10 (6H, t); MS 367.20 (M+H)⁺

### Example 51

### (R)-3-amino-4-(2-fluorophenyl)-N-((3R,4s)-1-methoxy-2-oxo-4-phenyl-1,2,3,4-tetrahydroquinolin-3-yl)butanamide

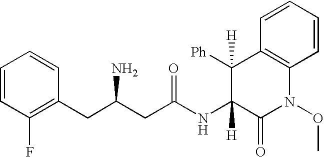

To a solution of (3R)-3-[(tert-butoxycarbonyl)amino]-4-(2-difluorophenyl)butanoic acid (193 mg, 0.65 mmol) in DCM (10 ml) was added sequentially EDAC (150 mg, 0.78 mmol), DIPEA (284 μl, 1.63 mmol), HOBt (105 mg, 0.78 mmol), and (3R,4S)-3-amino-1-methoxy-4-phenyl-3,4-dihydroquinolin-2(1H)-one hydrochloride (Intermediate 65; 239 mg, 0.65 mmol). The reaction mixture was allowed to stir at ambient temperature overnight. The reaction mixture was diluted with DCM and washed with 1M HCl and then aqueous sodium bicarbonate. The organic phase was concentrated under reduced pressure and the residue was purified by MPLC on silica (Isco Companion®; gradient elution from 100% iso-hexane to 100% ethyl acetate) to give the Boc protected title compound. This was taken up in 4M HCl in dioxane (5 ml) and stirred overnight at room temperature. The volatiles were removed and the residual oil loaded onto a Waters Oasis MCX cartridge, using methanol. Three column volumes of methanol were passed through the cartridge and discarded, followed by a further three column volumes of 10% NH₃ in methanol these were evaporated to give the title compound as the major component in a 78:22 mixture of diastereoisomers and as a colourless foam; (148 mg, 51%). ¹H NMR (CDCl₃): 2.05 (dd, 0.78 H), 2.20-2.84 (m, 3.22 H), 3.30 (m, 1H), 3.96 (s, 2.34 H), 4.00 (s, 0.66 H), 4.14 (d, 0.78 H), 4.69 (d, 0.22 H), 5.18 (m, 1H), 6.62 (d, 0.78 H), 6.92-7.40 (m, 12.22 H), 7.80 (d, 0.78 H), 7.94 (d, 0.22 H); MS 448 (M+H)⁺.

### Intermediate 65: (3R,4S)-3-Amino-1-methoxy-4-phenyl-3,4-dihydroquinolin-2(1H)-one Hydrochloride

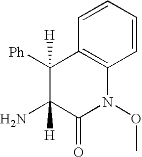

To a stirred solution of (R)-tert-butyl 1-(methoxyamino)-1-oxo-3,3-diphenylpropan-2-ylcarbamate (Intermediate 66; 1.00 g, 2.70 mmol) in DCM (10 ml) at 0° C. was added bis(triflouroacetoxy)iodobenzene (1.74 g, 4.05 mmol) in a single portion. The resulting solution was allowed to warm to room temperature and stirred overnight. The reaction mixture was diluted with DCM and washed with water and then saturated aqueous sodium bicarbonate and dried over magnesium sulphate. The organic phase was concentrated under reduced pressure and the residue was purified by MPLC on silica (Isco Companion®; gradient elution from 100% iso-hexane to 100% ethyl acetate) to give the N-Boc protected title compound as a pale yellow solid. This was taken up in 4M HCl in dioxane (5 ml) and stiffed overnight at room temperature. The volatiles were removed to give the title compound (0.71 g, 71%) as a colourless foam; ¹H NMR (CDCl₃): 3.88 (s, 3H), 4.70 (d, 1H), 4.86 (d, 1H), 6.44 (d, 1H), 7.04 (t, 1H), 7.24-7.54 (m, 7H), 8.64 (s, 2H); MS: 269 (M+H)⁺

### Intermediate 66: (R)-tert-butyl 1-(methoxyamino)-1-oxo-3,3-diphenylpropan-2-ylcarbamate

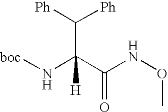

To a solution of Boc-L-3,3-diphenylalanine (2.32 g, 6.80 mmol) in DCM (50 ml) was added sequentially EDAC (1.960 g, 10.21 mmol), DIPEA (3.56 ml, 20.41 mmol), HOBt (1.37 g, 10.21 mmol), and O-methylhydroxylamine hydrochloride (1.14 g, 13.61 mmol). The reaction mixture was allowed to stir at ambient temperature overnight. The reaction mixture was diluted with DCM and washed with 1M HCl and then aqueous sodium bicarbonate. The organic phase was concentrated under reduced pressure and the residue was purified by MPLC on silica (Isco Companion®; gradient elution from 100% iso-hexane to 100% ethyl acetate) to give the title compound (2.11 g, 84%) as a colourless solid; ¹H NMR (CDCl₃): 1.24 (s, 9H), 3.30 (s, 3H), 4.42 (d, 1H), 4.65-4.83 (m, 1H), 5.12 (s, 1H), 7.03-7.38 (m, 10H), 9.02 (s, 1H); MS 393 (M+Na)⁺.

### Example 52

### (R)-3-Amino-4-(2-fluorophenyl)-N-((3S,4R)-1-methoxy-2-oxo-4-phenyl-1,2,3,4-tetrahydroquinolin-3-yl)butanamide

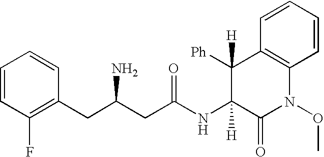

The title compound was prepared by the same method as for Example 51, starting from Boc-D-3,3-diphenylalanine.

¹H NMR: 1.92 (dd, 1 H), 2.04 (dd, 1 H), 2.70 (m, 2 H), 3.05 (m, 1H), 3.90 (s, 3 H), 4.43 (d, 1 H), 4.94 (d, 1 H), 6.56 (d, 1 H), 6.96 (t, 1 H) 7.04-7.40 (m, 12 H), 8.40 (d, 1 H); MS 448 (M+H)⁺.

### Example 53

### (3R)-3-Amino-4-(2-fluorophenyl)-N-[4-(4-fluorophenyl)-2-oxo-1,2-dihydroquinolin-3-yl]butanamide hydrochloride.

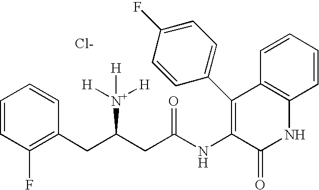

tert-Butyl((1R)-1-(2-fluorobenzyl)-3-{[4-(4-fluorophenyl)-2-oxo-1,2-dihydroquinolin-3-yl]amino}-3-oxopropyl)carbamate (Intermediate 67; 330 mg, 0.62 mmol) was treated with 4M HCl in dioxane (10 ml). The mixture was stirred overnight at room temperature and the dioxane evaporated under reduced pressure to give the (3R)-3-amino-4-(2-fluorophenyl)-N-[4-(4-fluorophenyl)-2-oxo-1,2-dihydroquinolin-3-yl]butanamide hydrochloride (292 mg, 0.62 mmol) as a solid.

¹H NMR: 2.21 (2H, m), 2.70 (2H, m), 7.05 (1H, d), 7.10-7.23 (5H, m), 7.31 (6H, m), 7.40-7.58 (2H, m), 8.20 (3H, s); LCMS (ESI+) 434 [M+H]⁺

### Intermediate 67: tert-Butyl((1R)-1-(2-fluorobenzyl)-3-{[4-(4-fluorophenyl)-2-oxo-1,2 dihydroquinolin-3-yl]amino}-3-oxopropyl)carbamate

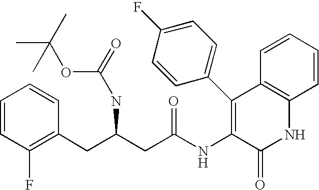

A mixture of (3R)-3-[(tert-butoxycarbonyl)amino]-4-(2-fluorophenyl)butanoic acid (Intermediate 68; 1.01 g, 3.4 mmol), 4-methylmorpholine (0.373 ml, 3.4 mmol) and 3-amino-4-(4-fluorophenyl)quinolin-2(1H)-one (864 mg, 3.4 mmol) in THF (20 ml) was treated with DMTMM (938 mg, 3.4 mmol). The mixture was stirred overnight at room temperature. The solvent was evaporated under reduced pressure and the product extracted with Et₂O. The desired product precipitated from the Et₂O and was isolated by filtration (330 mg, 0.62 mmol, 18%). LCMS (ESI+) 434 [M-Boc]⁺

### Intermediate 68: 3-Amino-4-(4-fluorophenyl)quinolin-2(1H)-one

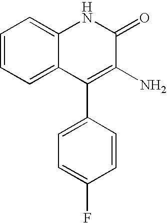

N-[4-(4-Fluorophenyl)-2-oxo-1,2-dihydroquinolin-3-yl]acetamide (Intermediate 69; 8.67 g, 0.029 mol) was dissolved in a mixture of acetic acid (20 ml) and sulfuric acid (130 ml). The reaction was heated at 150° C. for two hours and allowed to come down to room temperature. It was then poured on ice (300 g) and adjusted to pH 9 with a sodium carbonate solution (30 g/100 ml). The crude precipitate was separated by filtration and dried in the oven to give the 3-amino-4-(4-fluorophenyl)quinolin-2(1H)-one (8.66 g) as a brown solid. LCMS (ESI+) 254 [M+H]⁺

### Intermediate 69: N-[4-(4-fluorophenyl)-2-oxo-1,2-dihydroquinolin-3-yl]acetamide

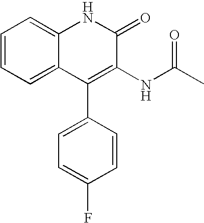

To N²-acetyl-N¹-[2-(4-fluorobenzoyl)phenyl]glycinamide (Intermediate 70; 7.23 g, 0.023 mol) in ethanol (150 ml) was added potassium tert-butoxide (2.83 g, 0.025 mol). The mixture was stirred at room temperature overnight. The ethanol was evaporated under reduced pressure to give the N-[4-(4-fluorophenyl)-2-oxo-1,2-dihydroquinolin-3-yl]acetamide (8.67 g) as a yellow solid. LCMS m/z (ESI+) 297 [M+H]⁺

### Intermediate 70: N²-acetyl-N¹-[2-(4-fluorobenzoyl)phenyl]glycinamide

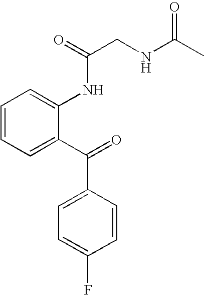

A solution of N-acetylglycine (2.96 g, 0.025 mol) in dry DCM was cooled to 0° C. was and treated with triethylamine (3.52 ml, 0.025 mol) followed by isobutyl chloroformate (3.27 mlL, 0.025 mol). The resulting mixture was stirred at −10° C. for 20 minutes and treated dropwise with a solution of (2-aminophenyl)(4-fluorophenyl) methanone (5 g, 0.023 mol) in DCM (20 ml) and then stirred at room temperature overnight. The mixture was extracted with DCM and washed successively with 0.1 M sodium hydroxide and water. The organic layer was dried over MgSO₄ and concentrated under reduced pressure to give the N²-acetyl-N¹-[2-(4-fluorobenzoyl)phenyl]glycinamide (7.23 g, 0.021 mol, 91%) as a solid. LCMS (ESI+) 337 [M+Na]⁺

1. A compound of formula (I) or a pharmaceutically-acceptable salt thereof, 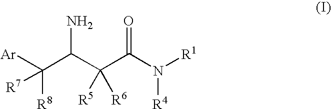
wherein:
Ar is phenyl optionally substituted with 1, 2, 3, 4 or 5 groups independently selected from R⁹;
R⁹ is selected from halo, (1-2C)alkyl optionally substituted with 1, 2, 3, 4 or 5 substituents independently selected from halo, hydroxy, methoxy optionally substituted with 1, 2 or 3 substituents independently selected from halo and cyano;
R¹ is selected from: 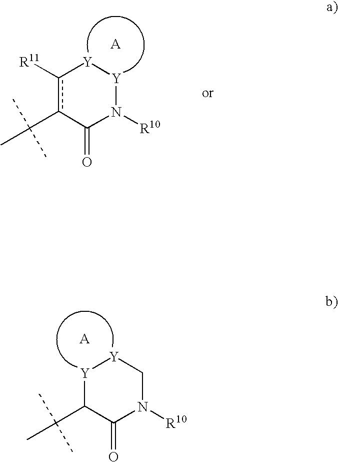
wherein [symbol] is a single or double bond;
R⁵ and R⁶ are independently selected from hydrogen, hydroxy and (1-4C)alkyl; or
R⁵ and R⁶ together with the carbon to which they are attached form a cyclopropyl ring;
R⁷ and R⁸ are independently selected from hydrogen, hydroxy and (1-4C)alkyl; or
R⁷ and R⁸ together with the carbon to which they are attached form a cyclopropyl ring;
provided that only one of R⁵, R⁶, R⁷ and R⁸ is hydroxy;
R⁴ is selected from hydrogen, (3-4C)cycloalkyl and (1-4C)alkyl optionally substituted with 1 substituent selected from (3-4C)cycloalkyl, hydroxy, (1-4C)alkoxy, halo and —S(O)p(1-4C)alkyl;
R¹⁰ is selected from hydrogen, (1-4C)alkyl, -(1-4C)alkyl(3-6C)cycloalkyl, hydroxy(1-4C)alkyl, (1-4C)alkoxy, (1-4C)alkoxy(1-4C)alkyl, (1-4C)alkylS(O)p(1-4C)alkyl, aryl(1-4C)alkyl, heteroaryl(1-4C)alkyl, -(1-4C)alkylCONH₂, -(1-4C)alkylCONH(1-4C)alkyl, -(1-4C)alkylCONdi(1-4C)alkyl, -(1-4C)alkylSO₂NH₂, -(1-4C)alkylSO₂NH(1-4C)alkyl, -(1-4C)alkylSO₂Ndi(1-4C)alkyl, -(2-4C)alkylNHCO(1-4C)alkyl, -(2-4C)alkylNHSO₂(1-4C)alkyl, -(1-4C)alkylCO₂H, and -(1-4C)alkylCO₂(1-4C)alkyl;
Y is carbon and Ring A is phenylene; or
each Y may independently be carbon or nitrogen and Ring A is 5- or 6-membered, heteroarylene ring containing 1 or 2 heteroatoms independently selected from O, N and S but not containing any O—O, O—S or S—S bonds, fused via Y as a ring carbon atom or nitrogen atom provided that the ring maintains aromaticity;
wherein Ring A is optionally substituted by 1 or 2 substituents independently selected from R²;
R² is independently selected from phenyl, heteroaryl, cyano, halo, (1-4C)alkyl, halo(1-4C)alkoxy, halo(1-4C)alkyl, dihalo(1-4C)alkyl, trifluoromethyl, pentafluoroethyl, (1-4C)alkoxy, hydroxy, amino, (1-4C)alkylamino, di(1-4C)alkylamino, —CONH₂, —CONH(1-4C)alkyl, —CONdi(1-4C)alkyl, —NHCO(1-4C)alkyl, —S(O)₂NH₂, —SO₂NH(1-4C)alkyl, —SO₂Ndi(1-4C)alkyl, —SO₂(1-4C)alkyl, -NHSO₂(1-4C)alkyl, —CO(1-4C)alkyl, —OCO(1-4C)alkyl, (3-5C)cycloalkyl, -(1-4C)alkyl(3-5C)cycloalkyl, halo(3-5C)cycloalkoxy, halo(3-5C)cycloalkyl, dihalo(3-5C)cycloalkyl, trihalo(3-5C)cycloalkyl, (3-5C)cycloalkoxy, (3-5C)cycloalkylamino, —CONH(3-5C)cycloalkyl, —NHCO(3-5C)cycloalkyl, —SO₂NH(3-5C)cycloalkyl, —SO₂(3-5C)cycloalkyl, —NHSO₂(3-5C)cycloalkyl, —CO(3-5C)cycloalkyl, —CO₂(3-5C)cycloalkyl and —OCO(3-5C)cycloalkyl;
R¹¹ is selected from hydrogen and phenyl optionally substituted by 1, 2 or 3 substituents independently selected from halo, (1-4C)alkyl, (1-4C)alkoxy, halo(1-4C)alkyl, halo(1-4C)alkoxy, (3-6C)cycloalkyl, (3-6C)cycloalkoxy, -(1-4)alkyl(3-6C)cycloalkyl, -(1-4C)alkoxy(3-6C)cycloalkyl, —S(O)p(1-4C)alkyl and —OSO₂(1-4C)alkyl;
p is independently at each occurrence 0, 1 or 2.

2. A compound of formula (I) as claimed in claim 1, or a pharmaceutically-acceptable salt thereof, wherein Ar is phenyl, optionally substituted with 1, 2 or 3 fluoro.

3. A compound of formula (I) as claimed in claim 1, or a pharmaceutically-acceptable salt thereof, wherein R¹ is

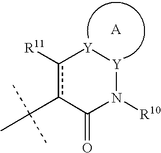

4. A compound of formula (I) as claimed in claim 1, or a pharmaceutically-acceptable salt thereof, wherein R⁵, R⁶, R⁷ and R⁸ are all hydrogen.

5. A compound of formula (I) as claimed in claim 1, or a pharmaceutically-acceptable salt thereof, wherein R⁴ is hydrogen.

6. A compound of formula (I) as claimed in claim 1, or a pharmaceutically-acceptable salt thereof, wherein:
Ar is phenyl optionally substituted with 1, 2 or 3 fluoro;
R⁴, R⁵, R⁶, R⁷ and R⁸ are hydrogen;
R¹ is 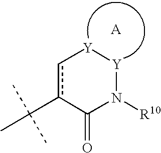
wherein [symbol] is a single or double bond;
A is pyridylene, optionally substituted by R²;
R¹⁰ is selected from hydrogen, optionally substituted benzyl, (1-4C)alkyl, (1-4C)alkyl, (3-6C)cycloalkyl(1-4C)alkyl, -(1-4C)alkylCO₂(1-4C)alkyl and (1-4C)alkoxy; and
R² is methoxy or trifluoromethyl.

7. A compound of formula (I) as claimed in claim 1, or a pharmaceutically-acceptable salt thereof, wherein:
Ar is phenyl optionally substituted with 1, 2 or 3 fluoro;
R⁴, R⁵, R⁶, R⁷ and R⁸ are hydrogen;
R¹ is 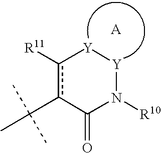
wherein [symbol] is a single or double bond;
A is pyridylene, optionally substituted by R²;
R¹¹ is phenyl, optionally substituted with fluoro;
R¹⁰ is selected from hydrogen, optionally substituted benzyl, (1-4C)alkyl, (1-4C)alkyl, (3-6C)cycloalkyl(1-4C)alkyl, -(1-4C)alkylCO₂(1-4C)alkyl and (1-4C)alkoxy; and
R² is methoxy or trifluoromethyl.

8. A compound of formula (I) as claimed in claim 1, or a pharmaceutically-acceptable salt thereof, which is a compound of formula (1A) or a pharmaceutically-acceptable salt thereof:

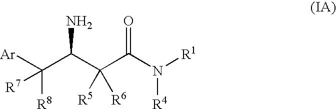

9. A compound of formula (I) as claimed in claim 1 or a pharmaceutically-acceptable salt thereof, which compound is any one or more of:
(3R)-3-amino-4-(2-fluorophenyl)-N-(2-oxo-1,2,3,4-tetrahydroquinolin-3-yl)butanamide;
(3R)-3-amino-4-(2-fluorophenyl)-N-(2-oxo-1,2,3,4-tetrahydro-1,5-naphthyridin-3-yl)butanamide;
(R)-3-amino-4-(2-fluorophenyl)-N-(2-oxo-1,2,3,4-tetrahydro-1,8-naphthyridin-3-yl)-butanamide dihydrochloride (and individual diasteroemers thereof);
(R)-3-amino-4-(4-fluorophenyl)-N-(2-oxo-1,2,3,4-tetrahydro-1,8-naphthyridin-3-yl)-butanamide;
(R)-3-amino-4-(2-fluorophenyl)-N-(2-oxo-1,2,3,4-tetrahydro-1,7-naphthyridin-3-yl)-butanamide;
(R)-3-amino-4-(4-fluorophenyl)-N-(2-oxo-1,2,3,4-tetrahydro-1,7-naphthyridin-3-yl)-butanamide;
(R)-3-amino-4-(2-fluorophenyl)-N-(2-oxo-1,2,3,4-tetrahydro-1,6-naphthyridin-3-yl)-butanamide;
(R)-3-amino-4-(4-fluorophenyl)-N-(2-oxo-1,2,3,4-tetrahydro-1,6-naphthyridin-3-yl)-butanamide;
(3R)-3-amino-4-(2-fluorophenyl)-N-(1-methyl-2-oxo-1,2,3,4-tetrahydro-1,5-naphthyridin-3-yl)butanamide dihydrochloride;
(3R)-3-amino-N-[1-(4-fluorobenzyl)-2-oxo-1,2,3,4-tetrahydro-1,5-naphthyridin-3-yl]-4-(2-fluorophenyl)butanamide dihydrochloride;
(3R)-3-amino-4-(2,5-difluorophenyl)-N-(2-oxo-1,2,3,4-tetrahydro-1,8-naphthyridin-3-yl)butanamide;
(3R)-3-amino-4-(2,5-difluorophenyl)-N-(2-oxo-1,2,3,4-tetrahydro-1,5-naphthyridin-3-yl)butanamide;
(3R)-3-amino-4-(2-fluorophenyl)-N-(2-oxo-1,2-dihydroquinolin-3-yl)butanamide hydrochloride;
(3R)-3-amino-4-(2-fluorophenyl)-N-(5-methoxy-2-oxo-1,2,3,4-tetrahydroquinolin-3-yl)butanamide hydrochloride;
(3R)-3-amino-4-(2,5-difluorophenyl)-N-[(3S)-2-oxo-1,2,3,4-tetrahydro-1,8-naphthyridin-3-yl]butanamide;
(3R)-3-amino-4-(2,5-difluorophenyl)-N-[(3R)-2-oxo-1,2,3,4-tetrahydro-1,8-naphthyridin-3-yl]butanamide dihydrogen chloride;
(3R)-3-amino-4-(2,4,5-trifluorophenyl)-N-[(3S)-2-oxo-1,2,3,4-tetrahydro-1,8-naphthyridin-3-yl]butanamide;
(3R)-3-amino-4-(2,4,5-trifluorophenyl)-N-[(3R)-2-oxo-1,2,3,4-tetrahydro-1,8-naphthyridin-3-yl]butanamide;
(3R)-3-amino-4-(2-fluorophenyl)-N-(1-methyl-2-oxo-1,2,3,4-tetrahydro-1,8-naphthyridin-3-yl)butanamide;
(3R)-3-amino-4-(2,5-difluorophenyl)-N-(2-oxo-1,2,3,4-tetrahydro-1,6-naphthyridin-3-yl)butanamide;
(3R)-3-amino-4-(2,5-difluorophenyl)-N-(2-oxo-1,2,3,4-tetrahydro-1,7-naphthyridin-3-yl)butanamide;
(3R)-3-amino-N-(1-ethyl-2-oxo-1,2,3,4-tetrahydro-1,8-naphthyridin-3-yl)-4-(2-fluorophenyl)butanamide;
(3R)-3-amino-N-[1-(cyclopropylmethyl)-2-oxo-1,2,3,4-tetrahydro-1,8-naphthyridin-3-yl]-4-(2-fluorophenyl)butanamide;
(3R)-3-amino-4-(2,5-difluorophenyl)-N-[1-(4-fluorobenzyl)-2-oxo-1,2,3,4-tetrahydro-1,8-naphthyridin-3-yl]butanamide;
(3S)-3-amino-3-(2,5-difluorophenyl)-N-[1-(4-fluorobenzyl)-2-oxo-1,2,3,4-tetrahydro-1,8-naphthyridin-3-yl]-N-methylpropanamide;
methyl [3-{[(3R)-3-amino-4-(2-fluorophenyl)butanoyl]amino}-2-oxo-3,4-dihydroquinolin-1(2H)-yl]acetate;
(3R)-3-amino-4-(2-fluorophenyl)-N-{1-[4-(methylsulfonyl)benzyl]-2-oxo-1,2,3,4-tetrahydro-1,5-naphthyridin-3-yl}butanamide dihydrochloride;
(3R)-3-amino-4-(2-fluorophenyl)-N-{2-oxo-1-[4-(trifluoromethoxy)benzyl]-1,2,3,4-tetrahydro-1,5-naphthyridin-3-yl}butanamide dihydrochloride;
(3R)-3-amino-4-(2-fluorophenyl)-N-{2-oxo-1-[4-(trifluoromethyl)benzyl]-1,2,3,4-tetrahydro-1,5-naphthyridin-3-yl}butanamide dihydrochloride;
(3R)-3-amino-4-(2-fluorophenyl)-N-{2-oxo-1-[4-(1,2,3-thiadiazol-4-yl)benzyl]-1,2,3,4-tetrahydro-1,5-naphthyridin-3-yl}butanamide dihydrochloride;
(3R)-3-amino-4-(2-fluorophenyl)-N-{1-[2-(4-methoxyphenyl)ethyl]-2-oxo-1,2,3,4-tetrahydro-1,5-naphthyridin-3-yl}butanamide dihydrochloride;
(3R)-3-amino-4-(2-fluorophenyl)-N-{1-[2-(4-fluorophenyl)ethyl]-2-oxo-1,2,3,4-tetrahydro-1,5-naphthyridin-3-yl}butanamide dihydrochloride;
methyl 4-{[3-{[(3R)-3-amino-4-(2-fluorophenyl)butanoyl]amino}-2-oxo-3,4-dihydro-1,8-naphthyridin-1(2H)-yl]methyl}benzoate;
(3R)-3-amino-N-[1-(3-chloro-4-methoxybenzyl)-2-oxo-1,2,3,4-tetrahydro-1,8-naphthyridin-3-yl]-4-(2-fluorophenyl)butanamide;
(3R)-3-amino-N-[1-(4-benzoylbenzyl)-2-oxo-1,2,3,4-tetrahydro-1,8-naphthyridin-3-yl]-4-(2-fluorophenyl)butanamide;
(3R)-N-{1-[4-(acetylamino)benzyl]-2-oxo-1,2,3,4-tetrahydro-1,8-naphthyridin-3-yl}-3-amino-4-(2-fluorophenyl)butanamide;
(3R)-3-amino-N-(6-fluoro-2-oxo-1,2,3,4-tetrahysroquinolin-3-yl)-4-(2-fluorophenyl)butanamide monohydrochloride;
(3R)-3-amino-N-(6-methoxy-2-oxo-1,2,3,4-tetrahysroquinolin-3-yl)-4-(2-fluorophenyl)butanamide;
(3R)-3-amino-4-(2-fluorophenyl)-N-(5-methyl-2-oxo-1,2,3,4-tetrahysroquinolin-3-yl)butanamide monohydrochloride;
(3R)-3-amino-4-(2,5-difluorophenyl)-N-(5-methoxy-2-oxo-1,2,3,4-tetrahysroquinolin-3-yl)butanamide;
(R)-3-amino-4-(2-fluorophenyl)-N-((3R,4S)-1-methoxy-2-oxo-4-phenyl-1,2,3,4-tetrahydroquinolin-3-yl)butanamide;
(R)-3-amino-4-(2-fluorophenyl)-N-((3S,4R)-1-methoxy-2-oxo-4-phenyl-1,2,3,4-tetrahydroquinolin-3-yl)butanamide; and
(3R)-3-amino-4-(2-fluorophenyl)-N-[4-(4-fluorophenyl)-2-oxo-1,2-dihydroquinolin-3-yl]butanamide hydrochloride.

10-12. (canceled)

13. A method for inhibiting DPP-IV in a warm-blooded animal, such as a human being, in need of such treatment which comprises administering to said animal an effective amount of a compound of formula (I) as claimed in claim 1, or a pharmaceutically-acceptable salt thereof.

14. A method of treating diabetes mellitus in a warm-blooded animal, such as a human being, in need of such treatment which comprises administering to said animal an effective amount of a compound of formula (I) as claimed in claim 1, or a pharmaceutically-acceptable salt thereof.

15-16. (canceled)

17. A pharmaceutical composition comprising a compound of formula (I) as claimed in claim 1, or a pharmaceutically-acceptable salt thereof, in association with a pharmaceutically-acceptable excipient or carrier.

18. A process for the manufacture of a compound of formula (I) as claimed in claim 1, or a pharmaceutically-acceptable salt thereof, said process comprising a process (a) to (c) as follows wherein the variables are as defined in claim 1 unless otherwise stated:
a) Coupling a compound of the formula (II) wherein P is a protecting group 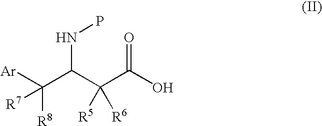
with a compound of the formula (IIIa) or (IIIb); 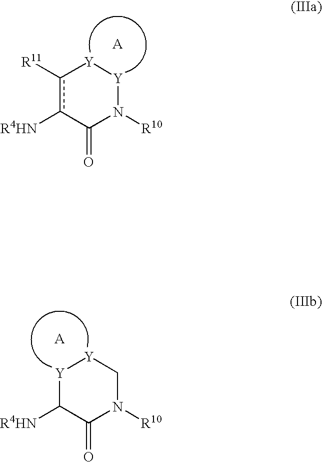
to give a compound of the formula (IVa) or (IVb); 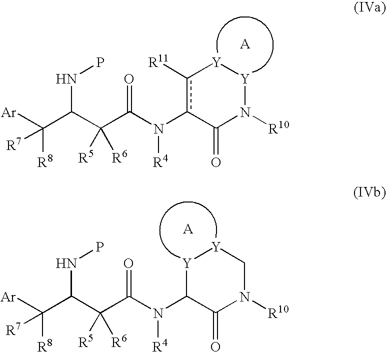
b) removing the protecting group P to give a compound of the formula (I);
c) optionally forming a pharmaceutically acceptable salt.
